SEQUENCE LISTING (38 sequences)

SEQ ID NO: 1 - protein, 4 aa, Artificial sequence
  Synthetic polypeptide
PGGS

SEQ ID NO: 2 - protein, 5 aa, Artificial sequence
  Synthetic polypeptide
PGGGS

SEQ ID NO: 3 - protein, 4 aa, Artificial sequence
  Synthetic polypeptide
PGGG

SEQ ID NO: 4 - protein, 4 aa, Artificial sequence
  Synthetic polypeptide
GGGG

SEQ ID NO: 5 - protein, 4 aa, Artificial sequence
  Synthetic polypeptide
PGGP

SEQ ID NO: 6 - protein, 4 aa, Artificial sequence
  Synthetic polypeptide
GGPG

SEQ ID NO: 7 - protein, 4 aa, Artificial sequence
  Synthetic polypeptide
GGSG

SEQ ID NO: 8 - protein, 4 aa, Artificial sequence
  Synthetic polypeptide
PGSG

SEQ ID NO: 9 - protein, 4 aa, Artificial sequence
  Synthetic polypeptide
PSSG

SEQ ID NO: 10 - protein, 4 aa, Artificial sequence
  Synthetic polypeptide
GSGG

SEQ ID NO: 11 - protein, 4 aa, Artificial sequence
  Synthetic polypeptide
PGSS

SEQ ID NO: 12 - protein, 4 aa, Artificial sequence
  Synthetic polypeptide
GSPS

SEQ ID NO: 13 - protein, 4 aa, Artificial sequence
  Synthetic polypeptide
GGSS

SEQ ID NO: 14 - protein, 4 aa, Artificial sequence
  Synthetic polypeptide
SSGS

SEQ ID NO: 15 - protein, 4 aa, Artificial sequence
  Synthetic polypeptide
SPSS

SEQ ID NO: 16 - protein, 4 aa, Artificial sequence
  Synthetic polypeptide
PGPG

SEQ ID NO: 17 - protein, 4 aa, Artificial sequence
  Synthetic polypeptide
GPGG

SEQ ID NO: 18 - protein, 7 aa, Artificial sequence
  Synthetic polypeptide
LEVLFXP

SEQ ID NO: 19 - protein, 4 aa, Artificial sequence
  Synthetic polypeptide
GPGS

SEQ ID NO: 20 - protein, 4 aa, Artificial sequence
  Synthetic polypeptide
GGPS

SEQ ID NO: 21 - protein, 5 aa, Artificial sequence
  Synthetic polypeptide
GPGGS

SEQ ID NO: 22 - protein, 5 aa, Artificial sequence
  Synthetic polypeptide
PGGSP

SEQ ID NO: 23 - protein, 5 aa, Artificial sequence
  Synthetic polypeptide
GGGSG

SEQ ID NO: 24 - protein, 5 aa, Artificial sequence
  Synthetic polypeptide
AGGGG

SEQ ID NO: 25 - protein, 6 aa, Artificial sequence
  Synthetic polypeptide
HHHHHH

SEQ ID NO: 26 - protein, 352 aa, Homo sapiens [sequence omitted]

SEQ ID NO: 27 - protein, 31 aa, Artificial sequence
  Synthetic polypeptide
MDYQVSSPIYDINYYTSEPCQKINVKQIAAR

SEQ ID NO: 28 - protein, 15 aa, Artificial sequence
  Synthetic polypeptide
HYAAAQWDFGNTMCQ

SEQ ID NO: 29 - protein, 31 aa, Artificial sequence
  Synthetic polypeptide
RSQKEGLHYTCSSHFPYSQYQFWKNFQTLKI

SEQ ID NO: 30 - protein, 17 aa, Artificial sequence
  Synthetic polypeptide
QEFFGLNNCSSSNRLDQ

SEQ ID NO: 31 - DNA, 399 bp, Artificial sequence
  Synthetic polynucleotide
atataagcttctcgagccgcaccatggattatcaagtgtcaagtccaatctatgacatcaattattatacatcggagccctgccaaaaaatcaatgtgaagcaaatcgcagcccgccccggcgggtctcactatgctgccgcccagtgggactttggaaatacaatgtgtcaacccggcgggtctagatctcaaaaagaaggtcttcattacacctgcagctctcattttccatacagtcagtatcaattctggaagaatttccagacattaaagatacccggcgggtctaggaattctttggcctgaataattgcagtagctctaacaggttggaccaacccggggggtctcatcatcatcatcatcattaggaattcaagcttgaagggcgaattcc

SEQ ID NO: 32 - DNA, 399 bp, Artificial sequence
  Synthetic polynucleotide
ggaattcgcccttcaagcttgaattcctaatgatgatgatgatgatgagaccccccgggttggtccaacctgttagagctactgcaattattcaggccaaagaattcctagacccgccgggtatctttaatgtctggaaattcttccagaattgatactgactgtatggaaaatgagagctgcaggtgtaatgaagaccttctttttgagatctagacccgccgggttgacacattgtatttccaaagtcccactgggcggcagcatagtgagacccgccggggcgggctgcgatttgcttcacattgattttttggcagggctccgatgtataataattgatgtcatagattggacttgacacttgataatccatggtgcggctcgagaagcttatat

SEQ ID NO: 33 - protein, 116 aa, Artificial sequence
  Synthetic polypeptide
MDYQVSSPIYDINYYTSEPCQKINVKQIAARPGGSHYAAAQWDFGNTMCQPGGSRSQKEGLHYTCSSHFPYSQYQFWKNFQTLKIPGGSQEFFGLNNCSSSNRLDQPGGSHHHHHH

SEQ ID NO: 34 - protein, 11 aa, Artificial sequence
  Synthetic polypeptide
PGGSGSFSSRT

SEQ ID NO: 35 - DNA, 1222 bp, Artificial sequence
  Synthetic polynucleotide
atataggatccgccgcaccatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggacctgaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcacccggggggtctggaagcttctcgagccgcaccatggattatcaagtgtcaagtccaatctatgacatcaattattatacatcggagccctgccaaaaaatcaatgtgaagcaaatcgcagcccgccccggcgggtctcactatgctgccgcccagtgggactttggaaatacaatgtgtcaacccggcgggtctagatctcaaaaagaaggtcttcattacacctgcagctctcattttccatacagtcagtatcaattctggaagaatttccagacattaaagatacccggcgggtctcaggaattctttggcctgaataattgcagtagctctaacaggttggaccaacccggggggtcttggatggagtgggacagagaaattaataattacacagggttaatatacaccttaattgaagaatcgcaaatccaacaagaaaagaatgaaaaagaattattggaattagataaatgggcaagtttgtggaattggtttaacataacaaattggctgtggtatataaaacccggggggtctcatcatcatcatcatcattaggaattcaagcttgaaggg

SEQ ID NO: 36 - DNA, 1222 bp, Artificial sequence
  Synthetic polynucleotide
cccttcaagcttgaattcctaatgatgatgatgatgatgagaccccccgggttttatataccacagccaatttgttatgttaaaccaattccacaaacttgcccatttatctaattccaataattctttttcattcttttcttgttggatttgcgattcttcaattaaggtgtatattaaccctgtgtaattattaatttctctgtcccactccatccaagaccccccgggttggtccaacctgttagagctactgcaattattcaggccaaagaattcctgagacccgccgggtatctttaatgtctggaaattcttccagaattgatactgactgtatggaaaatgagagctgcaggtgtaatgaagaccttctttttgagatctagacccgccgggttgacacattgtatttccaaagtcccactgggcggcagcatagtgagacccgccggggcgggctgcgatttgcttcacattgattttttggcagggctccgatgtataataattgatgtcatagattggacttgacacttgataatccatggtgcggctcgagaagcttccagaccccccgggtgccttctggaaagctagcaccacgatgtctattttgaactccaccttcttctggttctgcaagacagtgcatgtccaggtgccactatcctggagctccagctgagacacggagagggtcttccccccctgtatgtttttaccccttggactcctacattgcactgaggggctactaccaggggggctctccaaggtcagggtcaggctctgcccctgaagcaggtgggtgtcagagttggcagtcaatccgaacactagcaattgcacctcctccttcaggtcctccacttcacagatgtaagtatctgagtcttctatcttaagattcttgatgatcagggggaagtttccttggtcccaaaggcttcttcttgagtcagcgcgatcattcagcttggatggacctttagttaagaaggagccctgatttcccagaatctttatctggttggagtttttccagtggaattgtatgctcttcttctgggaagctgtacaggtcagttccactgtatccccttttttgcccagcaccactttctttccctgagtggctgctgggaggagcgccagttgcagcaccagaagcaagtgcctaaaagggactccccggttcatggtgcggcggatcctatat

SEQ ID NO: 37 - protein, 394 aa, Artificial sequence
  Synthetic polypeptide
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDLKEEVQLLVFGLTANSDTHLLQGQSLTLTLESPPGSSPSVQCRSPRGKNIQGGKTLSVSQLELQDSGTWTCTVLQNQKKVEFKIDIVVLAFQKAPGGSGSFSSRTMDYQVSSPIYDINYYTSEPCQKINVKQIAARPGGSHYAAAQWDFGNTMCQPGGSRSQKEGLHYTCSSHFPYSQYQFWKNFQTLKIPGGSQEFFGLNNCSSSNRLDQPGGSWMEWDREINNYTGLIYTLIEESQIQQEKNEKELLELDKWASLWNWFNITNWLWYIKPGGSHHHHHH

SEQ ID NO: 38 - protein, 4 aa, Artificial sequence
  Synthetic polypeptide
XXXX

(19) United States
(12) Patent Application Publication
(10) Pub. No.: US 2010/0249022 A1
(43) Pub. Date: Sep. 30, 2010

(54) STRATEGY FOR CLONING AND EXPRESSING THE EXTRACELLULAR DOMAINS OF RECEPTORS AS SOLUBLE PROTEINS

(75) Inventors: Paul Clapham, Paxton, MA (US); Alexander Repik, Northborough, MA (US)

Correspondence Address: WOLF GREENFIELD & SACKS, P.C., 600 ATLANTIC AVENUE, BOSTON, MA, 02210-2206, US

(73) Assignee: University of Massachusetts, Boston, MA (US)

(21) Appl. No.: 12/600,785

(22) PCT Filed: May 15, 2008
(86) PCT No.: PCT/US08/06199

§ 371 Date: May 12, 2010

Related U.S. Application Data
(60) Provisional application No. 60/930,910, filed on May 18, 2007.

(51) Int. Cl. A61K 38/16; C12N 5/02; G01N 33/53; A61P 35/00; A61P 27/02; C07K 14/00; C07K 16/00; C07H 21/04; A61P 31/18

(52) U.S. Cl. 514/3.8; 435/375; 436/86; 514/19.2; 514/20.8; 514/21.2; 530/350; 530/391.7; 536/23.1

(57) ABSTRACT

The present invention relates generally to soluble G protein-coupled receptor constructs. More specifically, the invention relates to soluble chemokine receptors, and soluble HIV co-receptors in particular. The invention is generally useful for designing and constructing soluble GPCR, which may be used to identify binding molecules. The invention also relates to methods of treating and/or preventing a disease or disorder associated with impaired function of such a receptor. The invention thus provides compositions and methods for therapeutic applications, such as vaccine.

### RELATED APPLICATION

This application claims priority under 35 U.S.C. §119 to U.S. Provisional Application No. 60/930,910, filed May 18, 2007, the entire contents of which is hereby incorporated by reference.

### GOVERNMENT SUPPORT

The work resulting in this invention was supported in part by NIH grants MH064408, AI062514 and HD049273. The U.S. Government may therefore be entitled to certain rights in the invention.

### FIELD OF THE INVENTION

The invention relates to methods of designing receptor fragments such as soluble G protein-coupled receptors. More specifically, the invention provides soluble chemokine receptor fragments and uses thereof. In particular, the compositions and methods of the present invention are useful for treating a subject afflicted with various disorders, for instance, a subject infected with the HIV-1 virus, and for preventing or retarding the manifestation of an HIV infection in a subject at risk or exposed to HIV.

### BACKGROUND OF THE INVENTION

G protein-coupled receptors (GPCRs) are involved in a wide variety of normal biological processes and in many pathological conditions (e.g. hypertension, cardiac dysfunction, depression, anxiety, obesity, inflammation, and pain). They are the target of 40-50% of modern medicinal drugs. Nevertheless, very little is known, at atomic resolution, about the detailed molecular mechanisms by which these membrane proteins are able to recognize their extracellular stimuli. Unfortunately, GPCRs, like other membrane-embedded proteins, have characteristics that make their 3D structure extremely difficult to determine experimentally. Therefore, the main ways to investigate the properties of GPCRs and its interaction with ligands are currently based on site-directed mutagenesis or molecular modeling techniques. In addition, for the vast majority of GPCRs, their cognate ligands has not yet been found.

Despite their structural similarities, GPCRs have been implicated in a wide range of biologically important functions. Malfunction of these receptors results in diseases, such as Alzheimer's Disease, Parkinson's Disease, diabetes, dwarfism, color blindness, retinal pigmentosa and asthma. GPCRs are also involved in depression, schizophrenia, sleeplessness, hypertension, anxiety, stress, renal failure as well as in several other cardiovascular, metabolic, neural, oncology and immune disorders (Horn, F. & Vriend G., 1998). Notably, the GPCRs, CCR5 and CXCR4 are implicated in HIV infection, where the receptors act as major coreceptors for viral entry into cells (Feng, Y., et al., 1996; Moser, B., 1997).

GPCRs confer a wide variety of physiological processes; (1) the visual sense: the opsins use a photoisomerization reaction to translate electromagnetic radiation into cellular signals, (2) the sense of smell: receptors of the olfactory epithelium bind odorants (olfactory receptors) and pheromones (vomeronasal receptors), (3) behavioral and mood regulation: receptors in the mammalian brain bind several different neurotransmitters, including serotonin and dopamine, (4) regulation of immune system activity and inflammation: chemokine receptors bind ligands that mediate intercellular communication between cells of the immune system; receptors such as histamine receptors bind inflammatory mediators and engage target cell types in the inflammatory response, (5) autonomic nervous system transmission: both the sympathetic and parasympathetic nervous systems are regulated by GPCR pathways. These systems are responsible for control of many automatic functions of the body such as blood pressure, heart rate and digestive processes.

It is well established that many medically important biological processes are mediated by proteins participating in signal transduction pathways that involve G-proteins and/or second messengers, e.g., cAMP (Lefkowitz, R. J., 1991). Activities of G-proteins themselves and their effector proteins are induced by signaling via GPCRs and include phospholipase C, adenylate cyclase, and phosphodiesterase, and actuator proteins, e.g., protein kinase A and protein kinase C.

Sequence comparison between different GPCRs revealed the existence of different receptor families sharing no sequence similarity. However, all these receptors have common structural features: an N-terminal extracellular domain, seven trans-membrane helices (TM-I through -VII) connected by three intracellular (ICL1, ICL2 and ICL3) and three extracellular (ECL1, ECL2 and ECL3) loops, and C-terminal intracellular domain. In addition, two cysteine residues (one in ECL1 and one in ECL2) are conserved in most GPCRs and form disulfide links, which are important for the packing and stabilization of a limited number of GPCR conformations. Five main families can be easily recognized when comparing their amino-acid sequences. Receptors from different families share no sequence similarity. Aside from sequence variations, GPCRs differ in the length and function of their N-terminal extracellular domain, their C-terminal intracellular domain and their intracellular loops. Each of these domains provide specific properties to these various receptor proteins.

Although the TM-7 alpha-helical domain plays a key role in ligand recognition and transduction, the importance of the extracellular loops for folding, ligand binding, and activation has also been demonstrated for many GPCRs, including GPCRs for trace amines, the opsin GPCRs for light, GPCRs for ‘peptides’ (neurokinin, angiotensin, etc.), and ‘protein’ (chemokine, glycoprotein hormone) receptors (Schwartz, T. W. & Rosenkilde, M. M., 1996; Metzger, T. G. et al., 1996; Doi, M. et al., 1990; Lerner, D. J. et al., 1996; Olah, M. E. et al., 1994; Walker, P. et al., 1994; Nanevicz, T. et al., 1996; Couture, L. et al., 1996; Fitzpatrick, V. D. & Vandlen, R. L., 1994). The amino acid sequences and lengths of the loops and the N-terminal fragment can vary widely. Typically, extracellular loops 1 and 3 (ECL1 and ECL3) are relatively short and merely connect transmembrane helices, while the N-terminal segment and ECL-2 are significantly longer. A large variety of molecular mechanisms allows the diverse ligands to activate the core domain.

All chemokine receptors have a common molecular architecture, which is conserved among family 1b G protein-coupled receptors (GPCRs). At a total length of 340 to 370 amino acids, they are composed of seven hydrophobic transmembrane domains with an extracellular N-terminal segment and a cytoplasmic C-terminal tail containing structural motifs which are critical for ligand-dependent signaling, desensitization, and receptor trafficking. Other structural features which are commonly found in chemokine receptors include cysteine residues in each of the four extracellular domains that form two disulfide bridges. These bridges probably impose a structural constraint on the extracellular receptor domains and thereby stabilize a receptor conformation which is capable of ligand binding. Chemokine receptors have classically been viewed as transducers of leukocyte chemoattractant peptides denoted as chemokines. Most of these peptides are secreted by many cell types in response to inflammatory stimuli (reviewed in: Hedrick, J. A. & A. Zlotnik, 1996; Baggiolini, M., 1998; Luster, A. D., 1998; Locati, M. & P. M. Murphy, 1999). Activation of chemokine receptors triggers an inflammatory response by inducing migration of leukocytes from the circulation to the site of injury and/or infection. However, chemokine and chemokine receptor-knock out experiments on mice have demonstrated that these molecules also play pivotal roles in angiogenesis, hematopoiesis, brain and heart development (Ma, Q. et al., 1998; Zou, Y. R. et al., 1998; Nagasawa, T. et al., 1996; Broxmeyer, H. E. & C. H. Kim, 1999; Baird, A. M., R. M. Gerstein & L. J. Berg, 1999). Furthermore, chemokine receptors have been identified as key coreceptors in the entry of HIV-1 into CD4+ cells (Feng, Y. et al., 1996; Deng, H. et al., 1996; reviewed in Berger, E. A., P. M. Murphy & J. M. Farber, 1999), thereby playing a major role in HIV-1 transmission and pathogenesis.

Chemokines are peptides, 70-120 residues long that are classified into four classes according to the location of the Cys residues at the N-terminus. The CXC class consists of chemokines with a pair of Cys separated by a single residue. The most prominent members of this class are interleukin-8 (IL-8, CXCL8), stromal derived factor-1 (SDF-1, CXCL12), gamma-interferon inducible protein-10 (IP-10, CXCL10), platelet factor-4 (PF-4, CXCL4), neutrophil activating protein-2 (NAP-2, CXCL7) and melanoma growth stimulating activity (MGSA, CXCL1). The CC class of chemokines have two adjacent Cys and include macrophage inflammatory protein-1 (MIP-1α, CCL3; MIP-1βa, CCL4), regulated upon activation of normal T expressed and secreted (RANTES, CCL5), monocyte chemoattractant protein-1 (MCP-1, CCL2). The CX3C class of chemokines contains two Cys separated by three residues and are represented by fractalkine/neurotactin (CX3CL1). The C-class chemokines contain a single Cys and are represented by lymphotactin/ATAC/SCM (CL1). Chemokine receptors are grouped according to their binding selectivity to chemokines. For example, CXCR1 binds IL-8, CXCR4 binds SDF-1 and CXCR5 binds B cell-attracting chemokine 1 (BCA1). CXCR2, CXCR3, and CCRs are promiscuous and bind several chemokines. For example, CCR5 binds MIP-1α, MIP-1β and RANTES.

Because of their biological, pharmacological and pathological importance, much effort has been made to study the function and structure of various GPCRs, particularly as potential therapeutic targets. Nevertheless, a technical hurdle has been that it is almost impossible to prepare soluble forms that retain the native structure for in vitro analysis of their interactions with ligands. Therefore, novel reagents that overcome the obstacle are of much interest.

### SUMMARY OF THE INVENTION

One aspect of the invention provides soluble polypeptides that retain three dimensional conformation of corresponding native GPCR proteins. The soluble GPCR polypeptide contains at least two extracellular domains of a GPCR protein, linked in tandem by short inter-domain linkers to form a soluble polypeptide that retains three dimensional conformation of a native GPCR corresponding to the extracellular domains of the GPCR protein. Because the overall topology of the extracellular portions of the receptor is intact, the soluble polypeptide is capable of binding a ligand or functionally equivalent analog for the native GPCR protein.

In some embodiments, the soluble GPCR polypeptide contains fragments of CCR5 or CXCR4 protein.

In some embodiments, a soluble GPCR polypeptide includes a tag, such as His⁶, GST and the like. Such tag may be on a carboxyl side of a soluble GPCR polypeptide, on an amino side of a soluble GPCR polypeptide, or both.

According to some embodiments of the invention, a soluble GPCR polypeptide includes a short, flexible peptide linker that comprises a proline and/or a hydrophobic amino acid, where preferred embodiments include a proline and a hydrophobic amino acid. In some embodiments, the peptide linker is PGGS [SEQ ID NO:1], PGGGS [SEQ ID NO:2], PGGG [SEQ ID NO:3], GGGG [SEQ ID NO:4], PGGP [SEQ ID NO:5], GGPG [SEQ ID NO:6], GGSG [SEQ ID NO:7], PGSG [SEQ ID NO:8], PSSG [SEQ ID NO:9], GSGG [SEQ ID NO:10], PGSS [SEQ ID NO:11], GSPS [SEQ ID NO:12], GGSS [SEQ ID NO:13], SSGS [SEQ ID NO:14], SPSS [SEQ ID NO:15], PGPG [SEQ ID NO:16], GPGG [SEQ ID NO:17], or XXXX (where X is selected from P, G and S) [SEQ ID NO:38].

In some embodiments, a soluble GPCR polypeptide comprising fragments of CCR5 protein is described. For example, a soluble CCR5 polypeptide may contain at least a portion of an N-terminal domain, an ECL1 domain, an ECL2 domain, and an ECL3 domain of CCR5, where each of the four domains is linked in tandem by a PGGS inter-domain linker. In some cases, each of the inter-domain linkers is PGGS. In other cases, a PGGS linker as well as other linker sequences may be used in a single soluble GPCR polypeptide.

In some embodiments, a soluble CCR5 polypeptide also includes one or more tags.

Similarly, soluble GPCR polypeptides derived from CXCR4 protein are provided. A soluble CXCR4 polypeptide may include at least a portion of an N-terminal domain, an ECL1 domain, an ECL2 domain, and an ECL3 domain of CXCR4, wherein each of the four domains is linked in tandem by a PGGS inter-domain linker. In some cases, each of the inter-domain linkers contained in a soluble CXCR4 polypeptide is a PGGS linker. However, in other cases, only a subset of the inter-domain linkers may be PGGS.

In some embodiments, a soluble CXCR4 polypeptide further comprises one or more tags.

The invention also provides compositions of a chimeric polypeptide comprised of a soluble GPCR polypeptide, at least a portion of CD4 N-terminal immunoglobulin variable region-like domain (e.g., D1 and D2 domains) and at least a portion of gp41 ectodomain (a C-terminal intramolecular interaction domain, such as the region corresponding to amino acid residues 628-683 of gp41, or an N-terminal intramolecular interaction domain), wherein each of the domains is linked in tandem by a short peptide linker.

In some embodiments, a chimeric polypeptide is comprised of a soluble GPCR and at least a portion of CD4 N-terminal immunoglobulin variable region-like domain (e.g., D1 and D2 domains), wherein each of the domains is linked in tandem by a short peptide linker. In other embodiments, a chimeric polypeptide is comprised of a soluble GPCR and at least a portion of gp41 ectodomain, wherein each of the domains is linked in tandem by a short peptide linker.

Another aspect of the invention is drawn to nucleic acids encoding soluble GPCR protein or their chimeric derivatives.

In some embodiments, the invention provides a nucleic acid encoding a soluble polypeptide that retains three dimensional conformation of a corresponding native GPCR protein. The soluble polypeptide according to the invention comprises at least two extracellular domains of a GPCR protein, linked in tandem by short inter-domain linkers to form a soluble polypeptide that retains three dimensional conformation of a native GPCR corresponding to the extracellular domains of the GPCR protein, wherein the soluble polypeptide is capable of binding a ligand or functionally equivalent analog thereof for the native GPCR protein. For example, the nucleic acid may encode a soluble polypeptide of CCR5, CXCR4, etc.

In some embodiments, the nucleic acid encodes a soluble polypeptide of CCR5 protein containing at least four domains comprising at least a portion of an N-terminal domain, an ECL1 domain, an ECL2 domain, and an ECL3 domain of CCR5, wherein each of the four domains is linked in tandem by a PGGS inter-domain linker [SEQ ID NO:1]. Similarly, the nucleic acid may encode CXCR4 protein comprising at least four domains comprising at least a portion of an N-terminal domain, an ECL1 domain, an ECL2 domain, and an ECL3 domain of CXCR4, wherein each of the four domains is linked in tandem by a PGGS inter-domain linker [SEQ ID NO:1].

In some embodiments, the nucleic acid of the invention further includes a nucleic acid encoding at least a portion of CD4 N-terminal immunoglobulin variable region-like domain and at least a portion of gp41 ectodomain, wherein each of the domains is linked in tandem by a nucleic acid encoding a short peptide linker.

In some embodiments, the nucleic acid is comprised of a nucleic acid encoding a soluble GPCR polypeptide coupled to a nucleic acid encoding at least a portion of CD4 N-terminal immunoglobulin variable region-like domain, wherein each of the domains is linked in tandem by a nucleic acid encoding a short peptide linker. Yet in other embodiments, the nucleic acid is comprised of a nucleic acid encoding a soluble GPCR polypeptide coupled to a nucleic acid encoding at least a portion of gp41 ectodomain, wherein each of the domains is linked in tandem by a nucleic acid encoding a short peptide linker. In some cases, the nucleic acid encoding the portion of CD4 N-terminal immunoglobulin variable region-like domain corresponds to D1 and D2 domains. In some cases, the nucleic acid encoding the portion of gp41 ectodomain corresponds to a C-terminal intramolecular interaction domain, e.g., amino acid residues 628-683 of gp41. Alternatively, the nucleic acid encoding the portion of gp41 ectodomain corresponds to an N-terminal intramolecular interaction domain.

In any of these embodiments, the nucleic acid encoding a soluble GPCR or derivatives (such as mutants and chimeras) thereof may further comprise a stretch of nucleic acid encoding a tag, such as His⁶-tag, biotin-tag, Glutathione-S-transferase (GST)-tag, Green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag. Chitin-tag, alkaline phosphatse (AP)-tag, HRP-tag, Biotin Carboxyl Carrier Protein (BCCP)-tag, Calmodulin-tag, S-tag, Strep-tag, haemoglutinin (HA)-tag, and digoxigenin (DIG)-tag, DsRed, RFP, Luciferase, Short Tetracysteine Tags, Halo-tag, Strep-tag, Nus-tag, as well as various other epitope tags that allow a single antibody to recognize specific protein.

The invention therefore further includes a vector plasmid comprising nucleic acid according to any one of the embodiments described herein. In addition, the invention includes a host cell expressing such vector plasmids. A number of commercially available vectors that are useful for using the instant invention incorporate a tag and/or other functional elements that may be used for facilitating the process of cloning, expression, purification, and so on. Non-limiting examples of such products include: 6xHIS Tag (Invitrogen, Life Technologies (Carlsbad, Calif., U.S.A.) Novagen (San Diego, Calif., U.S.A.), QIAGEN (Germany)); Calmodulin-Binding Peptide (CBP) Tag (Stratagene pCAL Vectors); Dihydrofolate Reductase (QIAGEN); Thioredoxin Fusion Sequences (Invitrogen, pTrxFUS and pThioHis Vectors, Novagen pET-32 Vectors); Protein A (Pharmacia pEZZ 18 and pRIT2T; Biotinylation (Promega PinPoint™ Vector)); Cellulose Binding Domain (CBD) (Novagen, pET CBD Vectors); Maltose Binding Protein (MBP) (New England BioLabs; Ipswich, Mass., U.S.A.; pMAL Vectors); S-Peptide Tag (Novagen, selected pET Vectors); Strep-tag (Biometra pASK75 Vector); Intein Mediated Purification with Affinity Chitin-Binding Tag (New England BioLabs, pCYB Vectors/IMPACT System); Immuno-reactive Epitopes (Invitrogen, Novagen, Kodak (Rochester, N.Y., U.S.A.)) Kinase Sequences for in vitro Labeling (Stratagene; La Jolla, Calif., U.S.A.; Pharmacia; Peapack, N.J., U.S.A.); ompT and ompA Leader Signal Peptides (Biometra; Germany; New England BioLabs; Kodak); malE Signal Sequence (New England BioLabs pMAL-p2); T7 gene 10 Leader Peptide (Novagen, Stratagene, Promega (Madison, Wis., U.S.A.), Invitrogen). Some commonly encountered protease/cleavage sites which may be used in conjunction with the instant invention are: Thrombin, Factor Xa Protease, Enterokinase, rTEV (a recombinant endopeptidase from the Tobacco Etch Virus). Intein-mediated self-cleavage (New England BioLabs), and 3C Human rhinovirus protease (Pharmacia Biotech) (Leu-Glu-Val-Leu-Phe-Gln/Gly-Pro) [SEQ ID NO:18].

According to a third aspect of the invention, methods for identifying a molecule that binds to a GPCR protein of native conformation are provided.

These screening methods include contacting a sample containing at least one test molecule with a soluble polypeptide that retains three dimensional conformation of a native GPCR protein or chimeric derivative thereof, comprising extracellular domains of a GPCR protein, linked in tandem by short inter-domain linkers, wherein the soluble polypeptide retains three dimensional conformation of the native GPCR, and subsequently identifying the molecule that binds to the polypeptide.

In some embodiments, the GPCR used in the method is an HIV co-receptor, such as CCR5 or CXCR4. In some cases, the soluble polypeptide used for screening further comprises one or more tags.

In some cases, the method of identifying a molecule that binds to a GPCR protein of native conformation may also include a separate step of isolating a molecule, prior to identifying the molecule that binds to the polypeptide. Depending on the assay system, the polypeptide may be immobilized to facilitate the screening.

The invention further contemplates embodiments where the screening is carried out in the presence and in the absence of a second factor, then comparing molecule(s) that bind to the polypeptide preferentially in the presence or in the absence of the second factor, and determining a co-factor.

The molecule that binds to a soluble GPCR of the present invention may be various types of molecules, such as an agonist, an antagonist, an inhibitor, a blocker, and a co-factor.

The screening method may be used to identify a molecule that is a binding agent or binding portion thereof, such as an antibody or a functionally equivalent fragment thereof. In some cases, the antibody is a conformation-specific antibody or a functionally equivalent fragment thereof. The invention contemplates that in some cases the screening is based on a high throughput assay.

In some embodiments, the method of the invention is used to screen for a small molecule that binds to a soluble GPCR polypeptide or derivative thereof. For example, the small molecule may be a naturally occurring small molecule, or a synthetic small molecule. In some circumstances, the molecule is a biosimilar.

The method is also useful for screening for molecules that bind to or inhibit the a virally encoded GPCR protein of interest that modulates host immune system. For example, the virally encoded GPCR protein may be encoded by a Herpes virus, Pox virus, and the like.

In addition, the method can be used to screen for molecules that may bind to and regulate a naturally occurring mutant GPCR protein with altered activity. For example, the naturally occurring mutant GPCR protein may be a constitutively active mutant. Alternatively, the naturally occurring mutant GPCR protein is a mutant with a reduced activity.

The naturally occurring mutant GPCR proteins may include, but are not limited to: rhodopsin, CCK2R, cholecystokinin-B/gastrin receptor subtype 2, CXCR2, CCR 1, TSHR, receptor for thyrotropin, LHR, receptor for luteinizing hormone, Receptor for follicle-stimulating hormone, PTH-PTHrPR, receptor for parathyroid hormone/parathyroid hormone-related peptide, CaR, Calcium-sensing receptor.

According to another aspect of the invention, methods for inhibiting ligand-dependent receptor stimulation of a GPCR are provided. The method includes a step of contacting a cell expressing the GPCR on the cell surface with a soluble polypeptide that retains three dimensional conformation of a native GPCR protein comprising at least two extracellular domains of a GPCR protein, linked in tandem by short inter-domain linkers to form a soluble polypeptide that retains three dimensional conformation of a native GPCR corresponding to the extracellular domains of the GPCR protein, wherein the soluble polypeptide is capable of binding a ligand or functionally equivalent analog thereof for the native GPCR protein in an amount effective for inhibiting ligand-dependent receptor stimulation of a GPCR. In some cases, the soluble polypeptide further comprises one or more tags.

In some embodiments, the method provides a CCR5-derived soluble polypeptide comprising at least four domains comprising at least a portion of an N-terminal domain, an ECL1 domain, an ECL2 domain, and an ECL3 domain of CCR5, wherein each of the four domains is linked in tandem by a PGGS inter-domain linker in an amount effective for inhibiting ligand-dependent stimulation of CCR5.

In some embodiments, the method includes a CXCR4-derived soluble polypeptide comprising at least four domains comprising at least a portion of an N-terminal domain, an ECL1 domain, an ECL2 domain, and an ECL3 domain of CXCR4, wherein each of the four domains is linked in tandem by a PGGS [SEQ ID NO:1] inter-domain linker in an amount effective for inhibiting ligand-dependent stimulation of CXCR4.

According to yet another aspect of the invention, methods for treating or preventing an HIV infection are provided. Such method may include administering to a subject in need of such treatment a composition comprising a soluble polypeptide that retains native three-dimensional conformation of extracellular portions of an HIV co-receptor, comprising at least part of an N-terminus, an ECL1 domain, an ECL2 domain and an ECL3 domain of the HIV coreceptor, linked in tandem by inter-domain PGGS [SEQ ID NO:1] linkers and disulfide bonds, in a pharmacologically effective amount to inhibit envelope gp120/CD4-mediated HIV-1 entry.

Typically, the soluble GPCR polypeptide for treating HIV infection is derived from a HIV co-receptor, including CCR5 and CXCR4.

In addition, a chimeric derivative may be used. For example, the method can use the soluble polypeptide further comprising at least a portion of CD4 N-terminal immunoglobulin variable region-like domains (e.g., D1 and D2 domains) and at least a portion of a gp41 ectodomain, wherein each of the domains is linked in tandem by a short peptide linker. In some embodiments, the soluble polypeptide further comprises at least a portion of CD4 N-terminal immunoglobulin variable region-like domains (e.g., D1 and D2 domains), wherein each of the domains is linked in tandem by a short peptide linker.

In some embodiments, the soluble polypeptide further comprises at least a portion of gp41 ectodomain, wherein each of the domains is linked in tandem by a short peptide linker. In some cases, the portion of gp41 ectodomain comprises a C-terminal intramolecular interaction domain, e.g., corresponding to amino acid residues 628-683 of gp41. Optionally, the portion of gp41 ectodomain may comprise an N-terminal intramolecular interaction domain.

In a further aspect, the invention provides HIV vaccines. The vaccines contain a soluble polypeptide that retains three dimensional conformation of a native CCR5 protein, CXCR4 protein or combination thereof, comprising at least four domains (at least a portion of an N-terminal domain, an ECL1 domain, an ECL2 domain, and an ECL3 domain of the CCR5 or CXCR4), where each of the four domains is linked in tandem by short inter-domain linkers comprising a proline and a hydrophobic amino acid, and wherein at least one of the short inter-domain linkers is a PGGS inter-domain linker, and a polypeptide comprising a CD4 N-terminal immunoglobulin variable region-like domain comprising a gp120 binding site.

In some embodiments, the vaccine includes the CD4 N-terminal immunoglobulin variable region-like domain corresponding to D1 and D2 domains.

In certain embodiments, the vaccine contains the soluble polypeptide that retains three dimensional conformation of a native CCR5 protein, CXCR4 protein or combination thereof, which is linked to the polypeptide comprising a CD4 N-terminal immunoglobulin variable region-like domain via a short inter-domain linker. In certain cases, the vaccine contains the CD4 N-terminal immunoglobulin variable region-like domain corresponding to D1 and D2 domains.

In some embodiments, the vaccine further comprises a polypeptide of a gp41 ectodomain (a membrane-proximate region) containing at least one of HR1 and HR2 neutralizing epitopes.

Some embodiments provide vaccines containing the polypeptide of a gp41 ectodomain linked to the soluble polypeptide that retains three dimensional conformation of a native CCR5 protein, CXCR4 protein or combination thereof via a short inter-domain linker.

In some embodiments, the vaccine further comprises a polypeptide of gp120 comprising a co-receptor binding site (v3 loop, in particular).

In any of the embodiments, the invention contemplates that the vaccine may further comprise an adjuvant.

In a further aspect, the invention embraces methods for inducing an immune response in a subject. The method involves administering to the subject having an HIV infection a composition comprising a soluble polypeptide comprising at least a portion of an N-terminal domain, an ECL1 domain, an ECL2 domain, and an ECL3 domain of a CCR5 protein, CXCR4 protein or combination thereof, wherein each of the four domains is linked in tandem by short inter-domain linkers comprising a proline and a hydrophobic amino acid, and wherein at least one of the short inter-domain linkers is a PGGS [SEQ ID NO:1] inter-domain linker, and a soluble polypeptide comprising a CD4 N-terminal immunoglobulin variable region-like domain comprising a gp120 binding site.

A further aspect of the invention includes methods for inducing an immune response in a subject. The method involves administering to the subject at risk of an HIV infection a composition comprising a soluble polypeptide comprising at least a portion of an N-terminal domain, an ECL1 domain, an ECL2 domain, and an ECL3 domain of a CCR5 protein, CXCR4 protein or combination thereof, a polypeptide of a gp41 ectodomain (e.g., a membrane-proximate region) containing at least one of HR1 and HR2 neutralizing epitopes, wherein each of the four domains is linked in tandem by short inter-domain linkers comprising a proline and a hydrophobic amino acid, and wherein at least one of the short inter-domain linkers is a PGGS [SEQ ID NO:1] inter-domain linker, and, a polypeptide comprising a CD4 N-terminal immunoglobulin variable region-like domain comprising a gp120 binding site.

In another aspect, the invention provides methods for treating a disease or disorder caused by a GPCR mutation, wherein the GPCR mutation is associated with altered basal activity. For example, the disease or disorder is selected from the group consisting of: Congenital night blindness, Retinitis pigmentosa, Gastric carcinoid tumors, Kaposi's sarcoma, primary effusion lymphoma, Atherosclerosis, infections in immunocompromised patients, Adenoma or hyperplasia associated with hyperthyroidism, Male precocious puberty, Leydig cell tumor associated with male precocious puberty, Normal semen parameters despite hypophysectomy, ansen-type metaphyseal chondrodysplasia (dwarfism, hypercalcemia, hypophosphatemia, Autosomal dominant hypocalcemia.

In some embodiments, the GPCR mutation occurs in at least one of the following: rhodopsin, CCK2R, cholecystokinin-B/gastrin receptor subtype 2, CXCR2, CCR 1, TSHR, receptor for thyrotropin, LHR, receptor for luteinizing hormone, Receptor for follicle-stimulating hormone, PTH-PTHrPR, receptor for parathyroid hormone/parathyroid hormone-related peptide, CaR, Calcium-sensing receptor.

In some cases, the disease or disorder is a virally-induced disease or disorder that affects host immune system, wherein the disease or disorder is caused by a virally encoded homologue of a GPCR or a ligand for a GPCR. Examples of viruses include, but are not limited to: HIV virus, Herpes virus and Pox virus.

The invention provides compositions comprising a plurality of receptors domain linked in tandem by short inter-domain peptide linkers including at least one proline and two glycine residues (e.g., a PGGS [SEQ ID NO:1] linker) to form a soluble polypeptide that retains three dimensional conformation of the plurality of receptor domains.

The invention further provides methods for treating HIV infection, where the method comprises a pharmaceutical administration of a soluble polypeptide having three HIV-reactive domains linked in tandem by short inter-domain peptide linkers. For example, in some embodiments, HIV-reactive domains of a soluble polypeptide of the invention are linked via PGGS linkers. In some cases, at least some of HIV-reactive domains of a soluble polypeptide of the invention may be connected by disulfide bonds. Some embodiments of the invention comprise HIV-reactive domains that are derived from extracellular domains of a GPCR protein, CD4 N-terminal immunoglobulin variable region-like domain and/or gp41 ectodomain.

### BRIEF DESCRIPTION OF THE DRAWINGS

FIG. 1A provides a schematic representation of the design strategy of soluble extracellular domain based GPCRs analog. ExCCR5 protein design is shown.

Left: Predicted topology of the chemokine receptor CCR5, showing the membrane-spanning helices (cylinders 1-7), N-terminal region (N) [SEQ ID NO:27], extracellular loops, ECL1 [SEQ ID NO:28], ECL2 [SEQ ID NO:29] and ECL3 [SEQ ID NO″30[, respectively, and disulfide bonds (S).

Right: Soluble extracellular domain based analog of GPCR (exCCR5), N-terminal region, extracellular loops and C-terminal 6xHis-tag is attached via flexible, turn-like (PGGS [SEQ ID NO:1]) linkers (L1, L2, L3, L4) at each junction.

FIG. 1B provides an amino acid sequence alignment of full length of human CCR5 (Top; [SEQ ID NO:26]) and extracellular domain-based analog, exCCR5 (Bottom). Also included is a scheme of Extracellular Domain-based Design of exCCR5 with engineered domains (N-term, ECL1, ECL2, ECL3) and flexible short interdomain-turn linker PGGS [SEQ ID NO:1] peptides (L).

FIG. 1C provides the 3D structure of an Fv from a human IgM immunoglobulin, adopted from Fan et al., 1992.

FIG. 2 depicts a scheme of the PCR strategy to generate a fusion protein comprising four extracellular domains of a GPCR. (A) Design of extracellular domain-based gene of GPCR. The four extracellular domains (N-terminus, ECL1, ECL2, ECL3) attached via flexible, turn-like linkers (L1, L2, L3, L4) at each junction. (B) Two step PCR strategy is shown. First step: separate PCRs with two pairs of long primers, from which two primary PCR products 1a (N-terminus-ECL1) and fragment 1b (ECL2-ELC3) are synthesized, which contain compatible ends. Second step: these two fragments are joined in a second, overlap extension PCR which also simultaneously introduces the regulatory elements necessary for expression via outer primers 5-F and 6-R.

FIG. 3A provides a synthetic gene sequence encoding for the N-terminus and 3 extracellullar loops of human chemokine receptor CCR5 [SEQ ID NO:31]. The complementary strand is also shown [SEQ ID NO:32]. Peptide translation is aligned beneath the coding sequences [SEQ ID NO:33]. The gene encodes for N-terminus and 3 extracellullar loops (ECL1, ECL2, ECL3) of CCR5 and a C-terminal 6xHis tag sequence. Four amino acid (PGGS) linkers [SEQ ID NO:1] were chosen for connection of the N-terminus, ECL1, ECL2, ECL3 and 6xHis-tag sequences (boxed codons). Four long internal oligonucleotide primers are denoted by grey shading; oligonucleotides of the upper DNA sequence are read 5′ to 3′ (left to right) and the oligonucleotides of the lower DNA sequence are read 3′ to 5′ (left to right). Two short flanking primers are denoted by underlined lines. Restriction sites and the Kozak sequence are denoted in Italic font.

FIG. 3B provides an agarose gel image showing two PCR Products of the first PCR cycle are separated by agarose gel electrophoresis. Lane 1: PCR fragment 1 with primers 1-F and 2-R introduce flexible turn PGGS linker peptides [SEQ ID NO:1] and overlap between N-ter and ECL1 domains. Lane 2: PCR fragment 2 with Primers 3-F and 4-R introduce flexible turn PGGS [SEQ ID NO:1] linker peptides and overlap between ECL2 and ECL3 domains. Lane 3: products of the second PCR; Fragments 1 and fragments 2 from first PCR were purified and used as templates for an overlap extension PCR that also incorporated primers 5-F and 6-R for the introduction of the regulatory elements and a C-terminal His⁶-tag; and, Lane 4: Molecular Weight Marker.

FIG. 4 provides an SDS-PAGE image of GST-exCCR5-6xHis protein purified by GST agarose. Samples were analyzed by 15% SDS-PAGE. GST-exCCR5-6xHis protein was expressed as N-terminal GST-tagged fusion protein in Rosetta-gami™ strain of E. coli (Novagen) and then purified by glutathione-sepharose affinity chromatography, Factor Xa treatment resulted in the removal of the GST tag with anion-exchange chromatography removing Factor Xa. Lane M: protein molecular marker; lane 1: unpurified soluble protein extract after induction with 1 mM IPTG; lane 2: elution fraction from glutathione-sepharose with 10 mM gluthathione; and lane 3: purified protein extract after proteolytic digestion and removal of factor Xa protease.

FIG. 5 is an SDS-PAGE image of the exCCR5-6xHis, purified using nickel chromatography utilizing the incorporated C-terminal 6xHis-tag and eluted with 300 mM imidazole. Purity of the eluted protein was assessed using 4-20% SDS-PAGE after extensive dialysis against phosphate buffered saline (PBS)-10% glycerol, in order to remove the imidazole.

FIG. 6 provides an SDS-PAGE image showing ExCCR5-6xHis expression in CHO cells. The gene of exCCR5-6xHis was subcloned into the mammalian expression vector, phCMV-3 and stably transfected into CHO cells. Individual clones secreting CCR5 were selected by limiting dilution cloning. The product was immunoprecipitated from the supernatant with 2D7 (a conformation-dependent antibody against CCR5) and analyzed by SDS 4-20% PAGE.

FIG. 7 provides a set of images of protein immunoblot analysis. Soluble GST-exCCR5-6xHis samples were immunoprecipitated with the 2D7 (a conformation-dependent antibody against CCR5). After precipitation, the eluted protein was immunoblotted and developed using different anti-CCR5 mAbs, as indicated.

FIG. 8 shows an image of immunoblot analysis. Pull down experiments for the analysis of RANTES binding to GST-exCCR5-6xHis protein. GST-exCCR5-6xHis was immobilized with either glutathione-sepharose beads or Ni-NTA Magnetic Agarose Beads. Lane 1: GST-exCCR5-6xHis protein (immobilization through GST N-terminal tag) and negative control GST (Lane 4) were immobilized on glutathione-sepharose beads and incubated with RANTES. After washing the beads four times with TEN buffer (20 mM Tris, pH 7.4, 0.1 mM EDTA and 100 mM NaCl), the bound proteins were eluted by boiling in sample buffer and visualized by Western blot analysis. Lane 2: positive control RANTES; Lane 3: RANTES binding to GST-exCCR5-6xHis (immobilization through C-terminal tag).

FIG. 9 provides a set of immunoblot analyses, showing CD4-dependent binding of gp120 envelope protein to soluble analog of CCR5 chemokine receptor. Recombinant gp120 envelope protein from the CCR5-using HIV-1 viral isolate, BaL, was tested for binding to GST-exCCR5-6xHis in presence (lane 2: 0.25 pg sCD4; lane 3: 0.5 pg sCD4; lane 4: 1 pg sCD4) or absence of sCD4 (lane 1); and, lane 5: GST-negative control. Bound gp120 was then detected by Western blotting with a sheep anti-gp120 antibody. The same membranes were stripped and hybridized with a His-HRP mAb against CCR5 (bottom panel) to show equivalent CCR5 loading.

FIG. 10 provides a graph showing results from ELISA assay. Specific CD4-dependent binding of gp120 Env proteins to exCCR5. A binding assay was performed by adding an increasing amount of R5 (Bal) or X4 IIIB gp120 proteins to exCCR5 are immobilized on Ni plates in absence or in presence (500 ng/ml) of sCD4.

FIG. 11 is a schematic illustration of HIV-1 attachment and entry. Opportunities for intervention in the HIV fusion cascade. The multi-step process of HIV entry into a CD4+ cells, can be divided into three steps: (i) the envelope glycoprotein (gp120) binds to the CD4 receptor; (ii) The gp120-CD4 complex interacts a chemokine coreceptor (CCR5 or CXCR4) on target cells; and (iii) gp41 extends and its fusion domain penetrates the cell membrane. Further conformational changes in gp41 result in the formation of the fusion active six-helix bundle, resulting in fusion of viral and cull membranes. Entry inhibitors, according to their mode of action, can be divided into three categories: (1) CD4 attachments inhibitors; (2) coreceptor binding inhibitors; and (3) fusion inhibitors that target gp41. All steps in the fusion cascade are suitable targets for pharmacologic intervention.

FIG. 12 provides a schematic representation of the strategy to produce a soluble multitarget entry inhibitor protein chimera: (left) predicted topology of the chemokine receptor CCR5, showing the membrane-spanning helices (cylinders 1-7), N-terminal region (N), extracellular loops (ECL1, ECL2, ECL3) and disulfide bonds (S); (center) the N-terminal region, extracellular loops and C-terminal 6xHis-tag are attached via flexible, turn-like (PGGS [SEQ ID NO:1]) linkers (L1, L2, L3, L4) at each junction; and, (right) soluble multitarget entry inhibitor chimera consisting of three structural elements. Structural element 1: CD4 D1D2 (residues 1-207); structural element 2: soluble extracellular domain-based analog of CCR5; structural element 3: gp41 ectodomain (residues 628-683). Structural element 1 is attached to element 2 via an 11 amino acid, flexible turn-like linker (L5). Element 3 is joined to a 6xHis-tag via a PGGS linker (L6).

FIG. 13 provides the predictive scheme for multi-step inhibition of HIV entry. Three steps in the fusion cascade will be inhibited in turn by each of the three structural elements of the chimeric protein: (1) The CD4 N-terminal D1-D2 domains will mimic CD4 receptor and Inhibit attachment of virus to cells; (2) the soluble extracellular domain-based analog will inhibit binding to the coreceptor; and (3) the gp41 helical region will inhibit the formation of the fusion active six-helix bundle, resulting in inhibition of fusion between viral and cellular membranes. Each of these processes will effectively block HIV entry.

FIG. 14 provides a synthetic gene sequence (sense and antisense strands shown [SEQ ID NOs: 35 & 36]) encoding for the CD4_{D1D2}-exCCR5-gp41(628-683)-6xHis soluble chimeric protein (translated polypeptide shown [SEQ ID NO:37]). The D1D2 domains of CD4 are shown joined to exCCR5 via a flexible turn-like linker, PGGSGSFSSRT (L5) [SEQ ID NO:34].

FIG. 15 depicts a schematic, illustrating a strategy for large-scale screening of GPCRs antagonists or agonists.

### DETAILED DESCRIPTION OF THE INVENTION

As disclosed herein, the present invention contemplates generating a soluble GPCR construct that mimics the extracellular structure of its native GPCR protein, thereby retaining its ability to bind to ligands and to interact with its extracellular or cell-surface binding partners. The extracellular domains are linked in tandem by short flexible linkers that maintain the appropriate 3D conformation to enable functional use of the fusion proteins. The fusion proteins are useful as novel therapeutics as well as research tools, such as high throughput assay reagents.

Thus the present invention provides in some aspects compositions comprising extracellular domains of G protein-coupled receptors (GPCRs) in soluble form, that substantially retain native three-dimensional conformation of the extracellular portions of their corresponding GPCRs. The invention accordingly provides a strategy for cloning and expressing such polypeptides. As used herein, “G protein-coupled receptor,” “GPCR” or “GPCR protein” refers to a member of the family or subfamily of seven-transmembrane receptor proteins that can transduce an extracellular signal mediated by ligand binding into an intracellular signal an signal that involves G protein activation.

Members of the GPCR family are integral proteins that share overall structural similarities. Thus, the invention as disclosed herein can be applied to any members of the GPCR family of receptor proteins. Examples of GPCRs include but are not limited to: 5-HT1A, 5-HT1B, 5-HT1D, 5-HT1E, 5-HT1F, 5-HT2A, 5-HT2B, 5-HT2C, 5-HT4, 5-HT5A, 5-HT6, 5-HT7, M1, M2, M3, M4, M5, A1, A2A, A2B, A3, a1A, a1B, a1D, a2A, a2B, a2C, b1, b2, b3, AT1, AT2, BB1, BB2, BB3, B1, B2, CB1, CB2, CXCR1, CXCR2, CXCR3, CXCR4, CXCR5, CXCR6, CCR1, CCR2, CCR3, CCR4, CCR5, CCR6, CCR7, CCR8, CCR9, CCR10, CX3CR1, XCR1, CCK1, CCK2, D1, D2, D3, D4, D5, ETA, ETB, GAL1, GAL2, GAL3, motilin, ghrelin, H1, H2, H3, H4, CysLT1, CysLT2, BLT1, BLT2, OXE, ALX, LPA1, LPA2, LPA3, S1P1, S1P2, S1P3, S1P4, S1P5, MCH1, MCH2, MC1, MC2, MC3, MC4, MC5, NMU1, NMU2, Y1, Y2, Y4, Y5, NTS1, NTS2, d, k, m, NOP, OX1, OX2, P2Y1, P2Y2, P2Y4, P2Y6, P2Y11, PAF, PKR1, PKR2, PRRP, DP, EP1, EP2, EP3, EP4, FP, IP1, TP, PAR1, PAR2, PAR3, PAR4, sst2, sst5, sst3, sst1, sst4, NK1, NK2, NK3, TRH, UT, OT, V1A, V2, V1B, APJ, FFA1, FFA2, FFA3, GPBA, TSH, LH, FSH, GnRH, KiSS1, MT1, MT2, NPFF1, NPFF2, NPS, NPBW1, NPBW2, P2Y12, P2Y13, QRFP, RXFP1, RXFP2, RXFP3, RXFP4, TA1, TA3, TA4 and TA5.

In a cell, after or during synthesis multiple domains of a GPCR protein fold into a specific three-dimensional conformation, or native conformation. Accordingly, the term “native GPCR” as used herein refers to a GPCR polypeptide (full length or segments thereof) that assumes substantially native three-dimensional conformation of the extracellular portions of the corresponding GPCR such that the polypeptide is properly folded for achieving that elicits particular biological function, such as, binding to a ligand or other binding partner(s) and/or causing conformational changes in the receptor that result in interactions with downstream effector molecules and produce a cascade of signaling events. The term “substantially native” as used herein means that the 3D conformation of a folded polypeptide is identical or considerably close to a corresponding in vivo structure such that the functionality of the peptide fragment or fragments is effectively retained. For example, a GPCR polypeptide with a substantially native conformation may exhibit slight structural deviation from its native counterpart but is capable of binding a ligand with a similar affinity. In some cases, a GPCR polypeptide with a substantially native conformation may bind to its ligand or binding partner with an altered affinity (either increased or reduced) but the binding is effectively selective such that it is useful for a particular application of interest.

Thus, an important function of GPCR is interaction with a binding partner. A GPCR binding partner is referred to herein as a ligand or binding partner. A ligand may be naturally occurring or synthetic. For example, some ligands are small molecules. A ligand that binds to one or more of GPCRs may elicit an activating effect, inhibiting effect, or neutral effect, with regard to the corresponding receptor activity. Examples of GPCR ligands include, but are not limited to: 5-hydroxytryptamine, acetylcholine, adenosine, noradrenaline, adrenaline, anaphylatoxin C5a, C5a des Arg74, anaphylatoxin C3a, angiotensin, apelin, neuromedin B, gastrin-releasing peptide, bradykinin, cannabinoid, CXCL1, CXCL2, CXCL3, CXCL4, CXCL5, CXCL6, CXCL7, CXCL8, CXCL9, CXCL10, CXCL11 (eotaxin), CXCL12, CXCL13, CXCL14, CXCL15, CXCL16, macrophage derived lectin, CCL1, CCL2, CCL3, CCL4, CCL5 (RANTES), CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CX3CL1, XCL1, XCL2, cholecystokinin, gastrin, dopamine, endothelin 1, endothelin 2, endothelin 3, long chain carboxylic acids, carboxylic acids, acetate, bile acids, galanin, motilin, ghrelin, thyroid-stimulating hormone, luteinizing hormone, chorionic gonadotropin, follicle-stimulating hormone, gonadotrophin-releasing hormone, histamine, KiSS-1 gene product, leukotriene D4, leukotriene C4, leukotriene B4, 5-oxo-ETE, lipoxin A, lysophosphatidic acid, sphingosine 1-phosphate, melanin-concentrating hormone, a-melanocyte stimulating hormone, adrenocorticotropic hormone, g-melanocyte stimulating hormone, b-melanocyte stimulating hormone, melatonin, neuromedin U, neuropeptide FF, Neuropeptide S, neuropeptide W, neuropeptide B, neuropeptide Y, pancreatic polypeptide, neurotensin, N-formyl-L-Met-L-Leu-L-Phe (fMLP), nicotinic acid (low affinity), nicotinic acid (high affinity), b-endorphin, dynorphin A, b-endorphin, nociceptin/orphanin FQ, orexin A, orexin B, ADP, UTP, ATP, UDP, UDP-glucose, RF-amide P518 gene product, platelet-activating factor, prokineticins 1, prokineticins 2, prolactin-releasing peptide, prostaglandin D2, prostaglandin E2, prostaglandin F2a, prostacyclin, thromboxane A2, 11-dehydro-thromboxane B2, thrombin, serine proteases, relaxin, relaxin-3, INSL5, relaxin-3, somatostatin, (lyso)phospholipid mediators, substance P, neurokinin A, neurokinin B, b-phenylethylamine, tyramine, thyrotropin-releasing hormone, urotensin II, oxytocin, vasopressin, sphingosine 1-phosphate, neuropeptide head activator, lysophosphatidic acid, succinate, a-ketoglutarate, b-alanine, BAMS-22, cortistatin, RARRES2, resolvin E1, TIG2, estrogen, obestatin, oleoylethanolamide, and free fatty acids.

As used herein, the term “in tandem” refers to a series of linked segments of polypeptides or corresponding nucleic acid that are connected in a linear fashion one behind another, with a defined order and orientation.

The present invention in some aspects provides a strategy for cloning and expression of extracellular domains of G protein-coupled receptors (GPCRs) in soluble form. The present invention therefore describes a strategy for producing soluble GPCR forms that comprise two or more extracellular regions (ECLs) linker to one another. For instance, preparation of a fusion protein of the invention, exCCR5, is described in the examples and referred to herein as an example. The invention is not limited to exCCR5. It is simply used to exemplify fusion proteins of the invention. ExCCR5 is a soluble form of the HIV-1 coreceptor, CCR5. ExCCR5 is able to bind three different ligands that depend on the conformational integrity of CCR5. These include HIV-1 gp120, the chemokine RANTES and a CCR5-specific monoclonal antibody. The soluble forms of the CCR5 and other GPCRs have utility directly in therapeutic applications or in other applications such as diagnostics, research tools or even high throughput screening assays to identify receptor-specific (or ligand specific) molecules that may have application in a wide variety of diseases e.g. AIDS, multiple sclerosis, rheumatoid arthritis and schizophrenia. The soluble fusion proteins may also form the basis for the design of specific proteins inhibitors that will also have potential in the therapy of a wide range of diseases.

According to one aspect of the invention, a soluble polypeptide of a GPCR is constructed that comprises at least two of the extracellular domains of the GPCR protein to mimic the extracellular portions of the native GPCR and its ligand-binding function. One of GPCR receptors' main functions is to recognize and respond to a specific ligand, and in GPCR proteins these ligands bind, at least in part, to the extracellular portion or portions.

By definition, “an extracellular domain” is the part of the receptor that “sticks out” of the membrane on the outside of the cell or organelle. Thus, as used herein, “an extracellular domain of a GPCR” refers to a segment of the protein that is substantially exposed to the outside of a cell when expressed on cell surface. The extracellular domain used in the construct may be either the entire extracellular domain or a portion thereof that contributes to recognition of the ligand. Because the polypeptide chain of the GPCR crosses the bilayer seven times (i.e., 7TM), the external domain can comprise “loops” protruding out of the membrane, in addition to the N-terminus that is positioned extracellularly. Most, if not all, GPCR proteins resume this overall structure. Accordingly, “an extracellular domain” of a GPCR protein is predicted to localize substantially on the extracellular surface based on amino acid sequence analyses and corresponding alignment of related sequences. Extracellular domains of a GPCR include an N-terminus and extracellular loops (ECL1, ECL2, and ECL3). The often large N-terminus of the polypeptide is thought to be important in ligand recognition. In addition, at least one of the ECLs, typically ECL, may also directly participate in ligand binding.

Similarly, “an intracellular domain” refers to the portion of a GPCR that is predicted to localize substantially on the intracellular side, including intracellular loops (ICL1, ICL2 and ICL3) and a C-terminus. It is also referred to as the cytoplasmic portion. The term “transmembrane” on the other hand, refers to the portion of a GPCR protein that is substantially spanning, or buried within, the phospholipid membrane. It is understood by those skilled in the art that the predicted topology is approximate. For example, certain amino acid residues that are expected to be buried in the membrane may become exposed upon changes in receptor conformation, and vice versa.

As used herein, “a transmembrane domain” of a GPCR is a segment of the polypeptide that spans the lipid bilayer. The transmembrane domains of a GPCR are presumed to undergo a conformational change upon binding of appropriate ligand on its extracellular face, which exerts an effect intracellularly. In some cases, the transmembrane domain may contain at least part of the ligand binding site.

In some embodiments, the entire length of each of the extracellular domains of a GPCR is used. In other embodiments, at least a portion of each of the extracellular domains of a GPCR is used. In some embodiments, the membrane-spanning domains and intracellular domains of a GPCR protein are completely excluded. However, in some circumstances, a portion of a transmembrane or intracellular domain near the adjacent extracellular domain may be included in designing a soluble GPCR polypeptide of the invention.

It should be noted that in many cases the exact junctions between these domains (e.g., extracellular, transmembrane, and intracellular domains) are not known but are deduced from known structures of related family of proteins, for which the three dimensional structure has been solved at the atomic level by crystallography, together with other information available based, for example, on sequence alignment, CD plot, etc. Therefore, the exact amino acid residues at which each domain begins with and ends with, are approximate. In addition, because a GPCR protein undergoes conformational changes upon its activation and deactivation, it is possible that some of the amino acid residues especially near the junction between two domains may switch positions depending on the activation status of the receptor.

According to some embodiments, a soluble GPCR polypeptide of the invention is derived from the extracellular domains of the chemokine receptor family of GPCR proteins. Chemokine receptors comprise a large family of GPCR proteins and include: CXCR1, CXCR2, CXCR3, CXCR4, CXCR5, CXCR6, CCR1, CCR2, CCR3, CCR4, CCR5, CCR6, CCR7, CCR8, CCR9, CCR10, CX3CR1, and XCR1.

In one embodiment, the constructs of the invention are based on soluble polypeptides constructed from CCR5 or CXCR4. CCR5 is expressed on resting and activated T-lymphocytes with memory/effector phenotype, monocytes, macrophages, and immature dendritic cells (Blanpain, C. et al., 2002). CCR5 contains 352 amino acids with a calculated molecular mass of 40.6 kDa and shares 71% sequence identity with CCR2 (a closely related receptor), with most of the differences being located on the extracellular and cytoplasmic domains (Combadiere, C. et al., 1996; Raport, C. J. et al., 1996; Samson, M. et al., 1996). CCR5's normal physiologic activities involve binding and transducing signals mediated by CC-chemokines, including RANTES, MIP-1α and MIP-1β, with direct activation and trafficking of T cells and other inflammatory cells. As such, CCR5 plays an important role in mediating the inflammatory reaction of diseases such as rheumatoid arthritis and multiple sclerosis. The CC chemokine receptor CCR5 is a major coreceptor for the entry HIV-1 R5 viruses into cells.

CXCR4 is expressed in numerous tissues, such as peripheral blood leukocytes, spleen, thymus, spinal cord, heart, placenta, lung, liver, skeletal muscle, kidney, pancreas, cerebellum, cerebral cortex and medulla (in microglia as well as in astrocytes), brain microvascular, coronary artery and umbilical cord endothelial cells. CXCR4 is involved in hematopoiesis and in cardiac ventricular septum formation. It plays an essential role in vascularization of the gastrointestinal tract, probably by regulating vascular branching and/or remodeling processes involving endothelial cells. CXCR4 may also be involved in cerebellar development. In the CNS, CXCR4 may mediate hippocampal neuron survival. CXCR4 acts as a coreceptor for HIV-1 X4.

A soluble GPCR according to the invention may be generally referred herein to as “sexdGPCR” (for soluble extracellular domains of GPCR) or sometimes as exGPCR or soluble GPCR polypeptide or GPCR constructs. Accordingly, a soluble CCR5 polypeptide of the present invention is designated as “sexdCCR5” (for soluble extracellular domains of CCR5), or sometimes as “exCCR5.” These terms are used interchangeably. Similarly, a soluble CXCR4 polypeptide of the invention is designated as “sexdCXCR4” or as “exCXCR4,” which are also interchangeable. These polypeptides are described in more detail below.

In some embodiments, a soluble GPCR analog comprises extracellular domains of the chemokine receptor, CCR5. This soluble CCR5 analog is an exCCR5 and contains the following components: an N-terminal domain, extracellular loops (ECL1, ECL2, and ECL3) and C-terminal 6xHis-tag, each of which is attached via flexible, turn-like (PGGS [SEQ ID NO:1]) linkers at each junction; further comprising disulfide bonds (S) that covalently connect ECL1 and ECL2, N-terminus and ECL3. In case of human CCR5, the cysteine residues involved in these disulfide bonds correspond to Cys¹⁰¹, Cys¹⁷⁸, Cys²⁰ and Cys²⁶⁹.

In some cases, a sexdCCR5 further includes an N-terminal tag, such as GST.

In some embodiments of the invention, a soluble GPCR analog comprises extracellular domains of the chemokine receptor, CXCR4. This soluble CXCR4 analog is an exCXCR4 and contains the following components: an N-terminal domain, extracellular loops (ECL1, ECL2, and ECL3) and C-terminal 6xHis-tag, each of which is attached via flexible, turn-like (PGGS [SEQ ID NO:1]) linkers at each junction; further comprising disulfide bonds (S) that covalently connect ECL1 and ECL2, N-terminus and ECL3. In case of human CXCR4, the cysteine residues involved in these disulfide bonds correspond to Cys¹⁰⁹, Cys¹⁸⁶, Cys²⁸ and Cys²⁷⁴.

In some cases, a sexdCXCR4 further includes an N-terminal tag, such as GST.

CCR5 and CXCR4 chemokine receptors are the major coreceptors for the HIV-1 virus to enter target cells. As used herein “a co-receptor (or coreceptor)” in the present context of HIV infection means that it is a co-factor that mediates viral entry into a target cell. Therefore, compositions and methods of the invention, that comprise a soluble polypeptide of an HIV coreceptor are useful for the treatment of HIV infection, as well as for screening molecules that are potentially useful for such purposes. Accordingly, the invention provides a soluble polypeptide of an HIV co-receptor. In some embodiments, the extracellular domains of CCR5 are linked in tandem via short flexible peptide linkers (e.g., PGGS [SEQ ID NO:1]).

In preferred embodiments, the N-terminus of CCR5 corresponds approximately to amino acid residues 1-31. The ECL1, ECL2 and ECL3 of CCR5 correspond approximately to amino acid residues 88-102, 168-198, and 261-277, respectively. In some cases, the soluble CCR5 polypeptide further comprises a tag on N-terminal (preferably a His⁶ tag) or C-terminal end of the peptide. In other cases, the soluble CCR5 polypeptide includes a tag on both ends.

The same approach can be taken for CXCR4. The invention provides embodiments where a soluble CXCR4 polypeptide is constructed, which comprises the N-terminus, ECL1, ECL2, and ECL3 of the CXCR4 protein, connected in tandem via short flexible linkers (e.g., PGGS [SEQ ID NO:1]) so as to form a contiguous polypeptide that folded into a conformation capable of binding a corresponding ligand. In preferred embodiments, the soluble CXCR4 is comprised of the N-terminus, ECL1, ECL2, ECL3, each connected via a PGGS linker [SEQ ID NO:1]. However, in some cases, other structurally suitable linkers may be used.

The soluble GPCR polypeptide also includes short inter-domain linkers that connect each of the domains in tandem so as to form a contiguous polypeptide. The short inter-domain linkers are flexible and preferably peptide based linkers. The design of the linkers is important to the invention because the linkers must hold the linked domains in a 3 dimensional structure that is consistent with the native molecule to achieve the proper function. Several selection criteria for linker design include, but are not limited to: length, prediction of secondary structure, hydrophobicity, solvent accessibilities and protease sensitivity.

In general, desirable features for a linker for inter-connecting the multiple domains of an engineered GPCR polypeptide are: it (1) is sufficiently short in length; (2) possesses structural flexibility; and, (3) provides a sharp turn that is suitable for promoting a correct orientation amongst these domains. As used herein, “a short peptide linker” consists of a polypeptide that is typically about 3 to 18 amino acid residues in length. A preferred peptide linker is about 3 to 10 amino acid residues in length. More preferably, a peptide linker is 4, 5 or 6 amino acid residues in length. Relatively long linkers may present undesirable hindrance and prevent receptor domains from properly folding into a correct conformation, causing it to interfere with its desired function. On the other hand, very short linkers, e.g., 1 or 2 amino acid residues, may not provide sufficient length to form a loop or turn inter-connecting two domains of a construct and may cause structural restrictions in flexibility, again, increasing the probability of mis-folding of the receptor domains that are linked.

For purposes of peptide linkers, the term “flexible” or “flexibility” refers to a structural feature of the peptide that favors disordered configuration, i.e., not inclined to form defined secondary structures. This is due to the fact that a linker sequence with high propensity for forming alpha-helical or beta-strand structures would limit the flexibility of the fusion protein and consequently affect its functional activity.

In addition to structural flexibility, it is desirable that a short peptide linker for inter-connecting the multiple extracellular domains of a GPCR provides a sharp “turn.” Such a turn would help the GPCR fragments to assume a sufficiently compact configuration as to allow appropriate inter-domain interactions that are necessary for proper folding and thus function (e.g., binding activity) of the receptor. To this end, some embodiments of the invention provide short, flexible peptide linkers that include a proline residue (Pro). Proline is a preferred amino acid in both linker and loop regions. This is because proline residues cannot donate hydrogen bonds or participate comfortably in any regular secondary structure conformation. Thus, proline cannot fit into the regular structure of either alpha-helix or beta-sheet and is frequently a common “breaker” of secondary structure. This again supports the notion that linkers should follow an extended conformation and act as spacers to allow domains to fold independently. Proline is also usually involved in a tight turn found in a number of proteins.

Such short, flexible peptide linker suitable for the instant invention may contain a turn structure within the linker so as to facilitate the formation of a loop configuration of a GPCR fusion proteins described herein. As used herein, a “turn” means that a residue or residues within a peptide linker assumes an angled configuration such that it allows the peptide linker to bend thereby facilitating intramolecular interactions of certain peptide domains that are connected via the linkers.

Taking into account the general preferred features of a peptide linker as discussed above, some embodiments of the invention provide a short flexible peptide linker for connecting the extracellular domains of a GPCR, comprises a Pro residues followed by one or more small hydrophobic amino acid residues or more preferred linker or loop amino acids. “A hydrophobic amino acid” is defined as an amino acid of non-polar properties. Examples of hydrophobic amino acids include: Glycine (Gly or G), Alanine (Ala or A), Valine (Val or V), Leucine (Leu or L), Isoleucine (Ile or I), and Proline (Pro or P). Examples of further preferred amino acids can be determined based on the linker database which includes the composition of inter-domain linkers and intra-domain loops connecting secondary structures. The amino acid residues, Pro, Gly, Asp, Asn, His, Ser and Thr (in order of preference) are preferred in loop regions.

In some embodiments, a short flexible peptide linker comprises “PGGS” (Pro-Gly-Gly-Ser) [SEQ ID NO:1]. In some cases, the Pro residue may be positioned at the second, third, or the forth position of the peptide linker, provided that such linker sequence is predicted to have relatively low propensity for forming alpha-helical or beta-strand structures, in order to maximize the flexibility of the fusion protein and consequently its functional activity.

To illustrate the effect of the relative position of a Pro residue in a linker, variations of linker sequences that are derivatives of the PGGS [SEQ ID NO:1] linker were analyzed in the context of soluble CCR5 fusion protein, including GPGS [SEQ ID NO:19], GGPS [SEQ ID NO:20], GPGGS [SEQ ID NO:21]. Analysis of these example linkers with Macvector showed that moving proline from first position increased the probability of secondary structure forming with the upstream extracellular domain, which may reduce structural flexibility.

In some embodiments of the invention, a short flexible peptide linker includes a serine residue (Ser), e.g., PGGS [SEQ ID NO:1]. In some cases, following proline, the linker sequence of the embodiment includes only glycine and serine residues. Linkers composed of these amino acids (e.g. GGGSG [SEQ ID NO:23]) are proteolytically stable, highly flexible and also successful in sterically separating domains. PGGS [SEQ ID NO:1] and PGGSP [SEQ ID NO:22] are particularly preferred linkers of the invention because it renders the fusion protein less susceptible to proteolysis, which is important for scaled-up production in E. coli. The PGGS [SEQ ID NO:1] linker, provided herein, is particularly suitable, as compared to the conventional GGGSG [SEQ ID NO:23] linker, for constructing multi-domain polypeptides such as GPCR, because it incorporates a sharp turn configuration that facilitates the correct folding of the receptor fragments with respect to one another.

Thus, the invention provides strategic designing of intra-domain linkers connecting secondary structure elements of the soluble GPCRs. Some of the criteria for designing suitable linkers are discussed in further detail below.

One of the important considerations is the choice of linker sequence to be placed between the N-terminus and the extracellular loops. Control of structural flexibility is important for the proper functioning of a large number of proteins and multiprotein complexes. For each protein, linker regions may be determined by assessing the branching out from the domain boundaries as assigned by the Taylor algorithm (For details see: Taylor W. R., 1999). Linker assignment ended when the branches became buried within the core of a domain. Overall, the largest proportion of linker residues, 38.3%, adopt an alpha-helical secondary structure, 13.6% are in β-strands, 8.4% are in turns and the rest, 37.6%, are in coiled or bent secondary structures (George R. & Hering a J., 2003). According to compositional comparison, linker sets can be divided into helical, strand and loops connecting secondary structure as defined by DSSP (Kabsch & Sander, 1983).

Linkers can be arbitrarily divided into several sets based on their relative length: small (less than six residues), medium (between six and 14 residues) and large (greater than 14 residues). In general, small linkers show an average hydrophobicity, while large linkers are more hydrophilic. Small linkers have a low to average solvent accessibility compared to medium sized linkers and large linkers. The larger the linker the more exposed it will be. The short linkers show increased propensities for hydrophobic residues and decreased propensities for polar and acidic residues (George R. & Hering a J., 2003). Comparison of linkers and of protein loops connecting secondary structures shows that the composition of inter-domain linkers is distinct from intra-domain loops connecting secondary structures. General consensus is that residues Pro, Gly, Asp, Asn, His, Ser and Thr are preferred in loop regions. In contrast, Gly, Asp, Asn and Ser are generally the least preferred within inter-domain linkers, while His and Thr have no preference. Inter-domain linkers are likely to facilitate the folding of multidomain proteins and are thought to act as rigid spacers to prevent non-native interactions between domains that may interfere with correct domain folding. Proline shows a high preference in both linker and loop sets, but may play a different role in each. A proline residue within a loop (as in exCCR5) is likely to be involved in a tight proline turn.

Based on available information, as described above, intra-domain linkers may be strategically designed. Theoretically it is possible to design two types of intra-domain linkers for the soluble exGPCRs (i.e., sexdGPCRs); (1) linkers with super secondary structures helical hairpin (helix-turn-helix) motif which will be mimic part of full length of transmembrane helixes of GPCRs, or (2) flexible, lack regular secondary structure, turn like, linkers tightly connecting head to tail of four extracellular elements of GPCRs.

For designing and constructing a helix-turn-helix type of linker, many attempts to design monomeric helical hairpin motifs have failed to realize the desired folded conform (Balaram, P., 1999; Karle I L et al., 1991). In a helix-turn-helix type of linker, hydrophobic helices are generally connected by short linker sequences containing various coded/noncoded amino acids with a tendency to break continuous helix formation, e.g., -aminocaprionic acid, L-lactic acid, Gly-Pro, D-Phe-Pro, and Gly-Dpg (where Dpg is -di-n-propylglycine), etc. Recently, successful design of two helices connected by a highly flexible L-Ala-(Gly)₄ [SEQ ID NO:24] was reported (Ramagopal U. et al., 2001). There are two problems of the monomeric helical hairpin motif design: (1) rational design of the flexible linkers; and, (2) the optimization of weak interactions between helices (termed long-range interactions). To address some of these issues, the present invention has taken into account in the design of soluble sexdGPCRs helical hairpin linkers that would allow optimization of all interactions between 7 helixes with each conferring single stable folds.

For designing and constructing a beta-turn type of linker, structural requirements for design of intra-domain β-turn linkers in exGPCRs are contemplated. Despite the difference in length, the secondary structure of the helix-turn-helix or beta-turn types of linkers offer turns that can stabilize of interactions between secondary structure elements. Such a turn in a linker would bring the flanking domains in close proximity.

It is possible to design beta-turn linkers of different lengths and amino acid composition. For example, (X_{N}-beta-turn-X_{N}), where X is the preferred loop amino residues and N its number). But practically, small linkers are preferable since the folding of longer linkers is often unpredictable. The small linkers show an average hydrophobicity, a low to average solvent accessibility compared to medium size or large linkers. The more exposed, longer linkers are the more independent and may allow movement of the structural elements of sexdGPCRs. The larger linkers increase the probability of forming of secondary structure constraints for sexdGPCR folding and limits linker flexibility. In general, small single domain proteins easily reach a native conformation and have fast folding kinetics (Jackson, 1998; Baneyx et al., 2004). However, the folding of larger proteins is often more unpredictable (Cabrita et al., 2004). The rate determining step of the folding process is often proline isomerization or disulfide bond rearrangement (Georgiou et al., 1996).

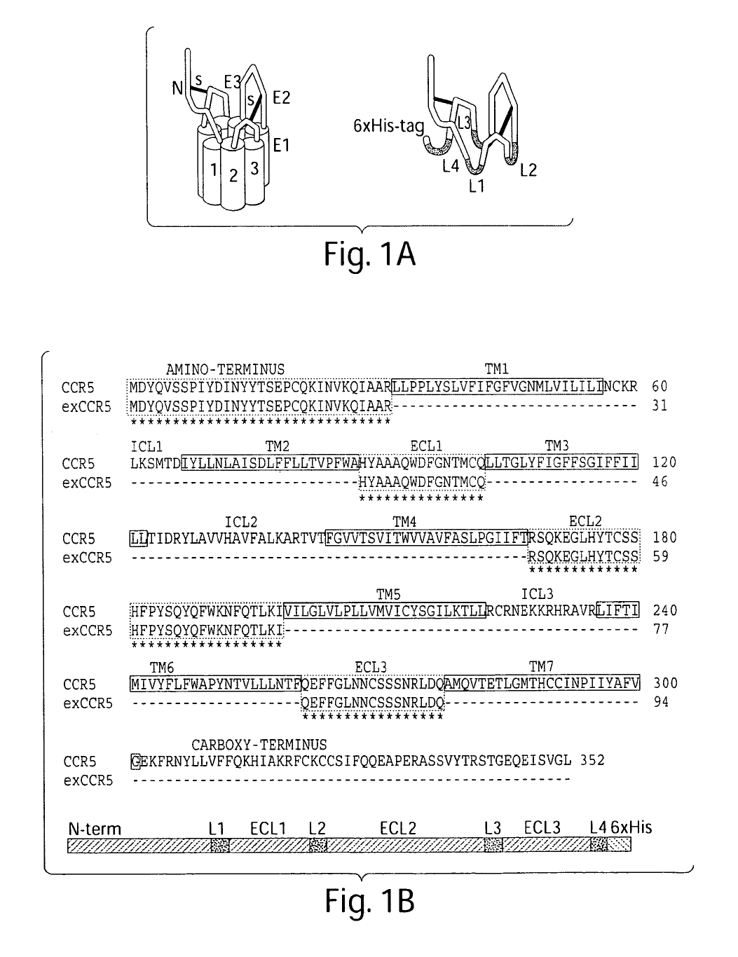

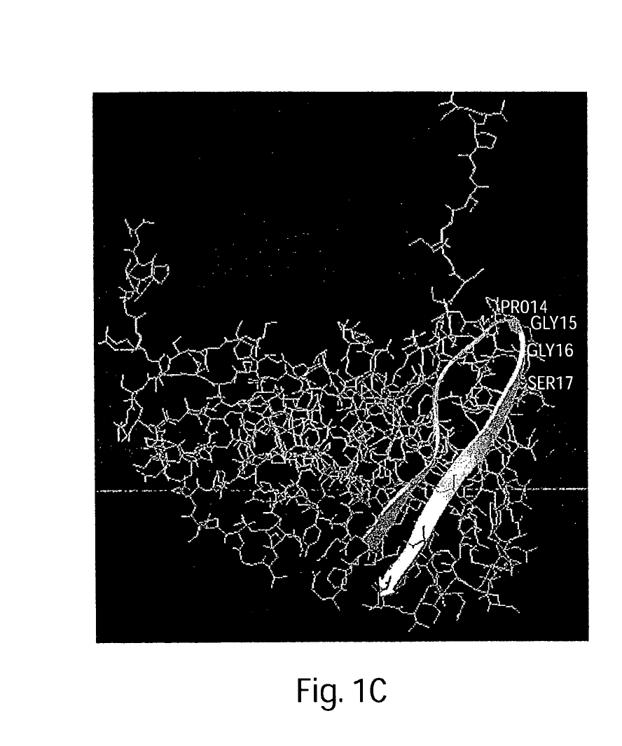

Based on these studies, the position of the putative linker regions in the context of sexdCCR5 can be analyzed using MacVector multiple prediction program runs. In addition, the LINKER program can be also used to generate linkers. This program is specially designed to assist in the construction of fusion proteins (J. Crasto & J Feng, 2000). The three dimensional structure of several natural intra-domain linkers was carried out with the aim to design independent linkers for gene fusion that would have a low likelihood of disrupting the folding of the flanking domains (Table 1). FIG. 1C shows the atomic-level structure of a natural 4 residue, PGGS [SEQ ID NO:1] beta-turn linker, which imposes a tight turn in FV from a human IgM immunoglobulin (Fan et al., 1992). Multiple selection criteria for linker design may be considered, including, for example, amino acid composition (to have minimal impact on sexdGPCRs folding), length, secondary structure prediction, hydrophobicity, solvent accessibility and protease sensitivity. Some of these factors in designing an inter-domain peptide linker are further discussed below.

Effects of amino acid composition for minimal impact on sexdGPCRs folding may be significant. According to the linker database, residues Pro, Gly, Asp, Asn, His, Ser and Thr are generally preferred residues in loop regions. The particularly preferred linker described in the present invention (Pro-Gly-Gly-Ser) [SEQ ID NO:1] includes three residues from this list. It consists of small amino acids thus minimizing possible conformation-constraints of amino acids with large side chains, or charged residues with the aim of limiting the impact of the linker composition on GPCRs folding. In some cases, a serine residue may be replaced with another amino acid residue, including but not limited to Thr and Met.

The position of the Proline residue (e.g., PGGS; [SEQ ID NO:1]) within a linker as well as with respect to the configurations of domains to be linked surrounding the linker is an important consideration. Proline is a preferred amino acid type in both linker and loop regions. It is particularly preferred at a first position of a linker. A linker sequence with a high propensity for forming α-helical or β-strand structures should be avoided, since these would limit the flexibility of the fusion protein and consequently affect its functional activity.

The inability of proline residues to donate hydrogen bonds or participate comfortably in any regular secondary structure conformation means they are usually involved in a tight turn. Proline is therefore a common “breaker” of secondary structure. The geometry allows a cis-proline to form a 180° turn in the polypeptide, which is known as a type VI or cis-Pro turn. Such a turn in a linker will bring the flanking domains in close proximity. Indeed, Analysis with MacVector shows that a Proline at the first position in the linker limits the likelihood the linker itself becomes incorporated into the secondary structure of previous region of the protein.

Length of linker is also a factor of consideration in designing a linker. A number of software programs are available to estimate the minimal length of linkers. Generally, one and two amino acid linkers pose structural restrictions, which result in limited flexibility. Therefore, linkers containing at least three, and in some instances, at least four amino acids are desirable.

Another factor to consider is the stability of a linker. As discussed elsewhere, amino acid residues such as Gly and Ser generally promote protease stability of a linker. For example, linkers composed of just these amino acids (GGGSG) [SEQ ID NO:23] have been reported to be proteolytically stable and highly flexible (Argos, 1990; Alfthan et al., 1995; Helfrich et al., 1998; Takeda et al., 2001). Accordingly, a preferred linker described herein contains a shorter variant of the GGGSG linker [SEQ ID NO:23], and should render the fusion protein less susceptible to proteolysis. The stability of protein is especially important for large-scale production of recombinant protein in E. coli.

Therefore, a preferred linker of the invention contain structural criteria including, but not limited to: (1) minimum length which approximately mimics the distance between transmembrane helices; (2) maximum flexibility; (3) turn-like type with proline in first position of linker which must also be a ‘breaker’ of secondary structure of previous extracellular loop and does not extend to next structural element of chimera; and (4) less susceptible to proteolysis.

Using the parameters described herein in combination with information obtainable from peptide prediction programs that are readily available, one of skill in the art can design additional suitable peptide linkers. Examples of such available programs include MacVector multiple prediction program and the LINKER program (Crasto C J & Feng J A, 2000). A number of studies have been conducted describing linker engineering and characterization of various linkers; see for example: Crasto C J, Feng J A (2000), Protein Eng. 13(5):309-12; Alfthan, K., Takkinen, K., Sizmann, D., Soderlund, H. and Teeri, T. T. (1995) Protein Eng., 8, 725-731; Argos, P. (1990) J. Mol. Biol., 211, 943-958; Asplund, M., Ramberg, M. and Johansson, B., 1111-1118; Takeda, S., Kamiya, N., Arai, R. and Nagamune, T. (2001) Biochem. Biophys. Res. Commun., 289, 299-304; Helfrich, W., Kroesen, B. J., Roovers, R. C., Westers, L., Molema, G., Hoogenboom, H. R. and de Leij, L. (1998) Int. J. Cancer, 76, 232-239.

It should be appreciated that the invention is useful for designing and constructing a wide variety of soluble polypeptides derived from multi-domain proteins, such as membrane receptors and channels. Accordingly, an aspect of the invention provides compositions comprising a plurality of receptor domains linked in tandem by short inter-domain peptide linkers that include at least one proline and one or more small hydrophobic amino acid residues (e.g., glycine) to form a soluble polypeptide that retains three dimensional conformation of the plurality of receptor domains. As used herein, “a receptor domain” refers to a structurally and/or functionally discrete segment of a native protein. For example, in case of a cell surface receptor, a receptor domain may correspond to an extracellular segment or a helical stretch that spans a membrane (i.e., transmembrane), and so on. In some embodiments, at least a subset of the inter-domain linkers that connect receptor domains comprises a PGGS linker [SEQ ID NO:1].

TABLE 1
Examples of intradomain linkers (LINKER software):

| | PDB | | |
|---|---|---|---|
| Loop sequences | access | Molecule conformations determined by X-ray | |
| of various lengths | code | crystallography or NMR | References |
| GGPG | 1A4U | ALCOHOL DEHYDROGENASE Chains: A, B | Benach, J. et al., 1998 |
| [SEQ ID NO: 6] | | EC no.: 1.1.1.1 | |
| GGSG | 1A9Z | UDP-GALACTOSE 4,EPIMERA-SE MUTANT S124A/Y149F | Thoden, J. B., 1998 |
| [SEQ ID NO: 7] | | COMPLEXED WITH UDP-GALACTOSE | |
| PGSG | 2AK3 | THE THREE-DIMENSIONAL STRUCTURE OF THE COMPLEX BETWEEN | Diederichs, K. et. al., |
| [SEQ ID NO: 8] | | MITOCHONDRIAL MATRIX ADENYLATE KINASE AND ITS | 1991 |
| | | SUBSTRATE AMP AT 1.85 ANGSTROMS RESOLUTION | |
| PSSG | 1AXZ | ERYTHRINA CORALLODEN-DRON LECTIN IN COMPLEX WITH | Elgavish, S., 1998 |
| [SEQ ID NO: 9] | | D-GALACTOSE | |
| GSGG | 2BVV | SUGAR RING DISTORTION IN THE GLYCOSYL-ENZYME | Sidhu, G., et al., 1999 |
| [SEQ ID NO: 10] | | INTERMEDIATE OF A FAMILY G/11 XYLANASE. | |
| PGSS | 1CFT | ANTI-P24 (HIV-1) FAB FRAGMENT CB41 COMPLEXED WITH AN | Keitel, T. et. al., 1997 |
| [SEQ ID NO: 11] | | EPITOPE-UNRELATED D-PEPTIDE | |
| GSPS | 2ERK | PHOSPHORYLATED MAP KINASE ERK2 | Canagarajah, B. et. |
| [SEQ ID NO: 12] | | | al., 1997 |
| GGSS | 1HKC | RECOMBINANT HUMAN HEXOKINASE TYPE I COMPLEXED WITH | Aleshin, A. et al., |
| [SEQ ID NO: 13] | | GLUCOSE AND PHOSPHATE | 1998 |
| PGGS | 1IGM | THREE DIMENSIONAL STRUCTURE OF AN FV FROM A HUMAN IGM | Fan, Z. et al., 1992 |
| [SEQ ID NO: 1] | | IMMUNOGLOBULIN | |
| SSGS | 1IVY | PHYSIOLOGICAL DIMER HPP PRECURSOR | Rudenko, G. et al., |
| [SEQ ID NO: 14] | | | 1995 |
| SPSS | 1KBA | CRYSTAL STRUCTURE OF KAPPA-BUNGAROTOXIN AT 2.3- | Dewan, J. C. et al., |
| [SEQ ID NO: 15] | | ANGSTROM RESOLUTION | 1994 |
| PGPG | 2NEF | HIV-1 NEF (REGULATORY FACTOR), NMR, 40 STRUCTURES | Grzesiek, S. et al., |
| [SEQ ID NO: 16] | | | 1997 |
| GPGG | 1REF | ENDO-1,4-BETA-XYLANASE II COMPLEX WITH 2,3- | Havukainen, R. et |
| [SEQ ID NO: 17] | | EPOXYPROPYL-BETA-D-XYLOSIDE | al., 1996 |

Accordingly, the invention provides compositions of a soluble GPCR polypeptide comprising at least two or three extracellular domains of the GPCR protein linked in tandem via short flexible peptide linkers so as to form a contiguous polypeptide, such that the polypeptide contains, in order from the amino terminus to the carboxyl terminus: an extracellular domain-linker-extracellular domain. In some embodiments the fusion protein includes an N-terminal extracellular segment of a GPCR, a linker, an ECL1, a linker, an ECL2, a linker, and an ECL3. According to some embodiments of the invention, a soluble GPCR polypeptides include a portion of each of the extracellular fragments linked in tandem via peptide linkers. In other embodiments, a soluble GPCR polypeptide of the invention includes complete extracellular domains linked in tandem via peptide linkers. Yet in other embodiments, a soluble GPCR polypeptide of the invention includes one or more partial fragments of extracellular domains and one or more complete extracellular domains linked in tandem via peptide linkers.

Thus, an important consideration in the design of sexdCCR5 and sexdCXCR4 described in the examples was the choice of linker sequence to be placed between the extracellular domains. The selected linker PGGS [SEQ ID NO:1] corresponds to the selection criteria described herein; minimum length to approximately mimic the distance between transmembrane helixes in the native receptor, maximum flexibility and a turn-like structure with proline preferably in the first position. The linker is a ‘breaker’ of secondary structure and separates the upstream and downstream extracellular loops without becoming incorporated into their structure. PGGS linkers [SEQ ID NO:1] impose a tight turn between extracellular domains in the sexdCCR5 chimera.

In some embodiments, one or more tags may be included. For example, some compositions as described herein include an N-terminal tag. Similarly, some embodiments provide compositions that include a C-terminal tag. Yet in other embodiments, compositions include both an N-terminal and a C-terminal tags. As used herein, “a C-terminal tag” refers to a carboxyl tag. A carboxyl tag is placed at the end of a polypeptide chain (i.e., the only amino acid residue in a polypeptide chain with a free α-carboxyl group) defined as the carboxyl terminus of the polypeptide. As used herein, “an N-terminal tag” is an amino tag is a tag, which is placed at the beginning of a polypeptide chain of interest, in which “amino” refers to the only amino acid residue in a polypeptide chain with a free α-amino group. Examples of tags include but are not limited to: a His⁶ (or 6xHIS) tag [SEQ ID NO:25], a biotin tag, a Glutathione-S-transferase (GST) tag, a Green fluorescent protein (GFP) tag, a c-myc tag, a FLAG tag, a Thioredoxin tag, a Glu tag, a Nus tag, a V5 tag, a calmodulin-binding protein (CBP) tag, a Maltose binding protein (MBP) tag. a Chitin tag, an alkaline phosphatse (AP) tag, an HRP tag, a Biotin Carboxyl Carrier Protein (BCCP) tag, a Calmodulin tag, an S tag, a Strep tag, a haemoglutinin (HA) tag, a digoxigenin (DIG) tag.

As discussed above, for the GPCR constructs of the invention to exert proper cellular function, it is necessary that the protein is folded correctly such that it substantially retains native three-dimensional conformation. As used herein, “native three-dimensional conformation” means that a GPCR polypeptide is folded virtually identical to or substantially similar to the structure of the native full length GPCR counterpart so as to preserve the ability to elicit a particular biological function associated with the native GPCR protein. As used herein, “substantially” means that the structure of two or more comparative molecules or portions of a molecule associated with a particular function are sufficiently intact that biological function is retained. As used herein, “biological function” refers to any activities of a molecule, such as a protein, a fragment thereof and a complex thereof, including enzymatic/catalytic activities and specific binding activities.

The compositions of the invention are soluble molecules. As used herein, the term “soluble” refers to a biochemical characteristic of an expressed protein or polypeptide that is readily isolated from the subcellular fractions that are either cytosolic or readily extractable (e.g., without the use of harsh detergents). The solubility of a particular protein or polypeptide may be affected by a number of factors, including charge average, turn forming residue fraction, cysteine fraction, proline fraction, hydrophilicity, and total number of residues (size), pI, pH, overall structure (globular vs. fibril etc.), the presence of localization signal or solubility-enhancing tags, such as maltose binding protein (MBP), thioredoxin (Trx) and glutathione-S-transferase (GST), among others. In addition, there are several general fall-back strategies for expression of correctly folded eukaryotic proteins in E. coli one of which is to truncate long multi-domain proteins into separate domains. Reducing translation rates so that proteins have an increased chance of folding into a native state prior to aggregating with folding intermediates, can be successful by lowering the temperature after induction or inducing with lower concentrations of IPTG. Alternate approaches include: co-expressing stabilizing binding partners or chaperones; the induction of chaperones by heat shock or chemical treatment; or the use of genetically modified host-strains that can conduct oxidative protein folding in the cytoplasm, over-express rare tRNAs or lipid rafts.

The GPCR constructs of the invention may be produced recombinantly. A “recombinant” protein or polypeptide refers to a protein or polypeptide that has been induced to be expressed by transfection, infection or any other means of gene transfer, using an exogenous source of nucleic acid, e.g., recombinant cDNA operatively linked to an appropriate transcription system using a vector plasmid, in a host cell system suitable for expression. A number of “vector plasmids” are known in the art and may be used for the present invention. Examples of expression systems include but are not limited to: bacterial expression systems, such as E. coli; eukaryotic expression systems, such as yeast, insect cells (e.g., baculo-virus-mediated expression of SF9 cells); and various mammalian cells, such as COS-1, COS-9, 3T3, PC12, MDCK, HeLa, and many other heterologous cell systems. Alternatively, expression of recombinant proteins may be carried out in a cell-free system (i.e., in vitro translation). Recombinant proteins may also be expressed in tissue (i.e., in vivo gene transfer), either selective or systematic/general, or a subset of cell type, for example, for purposes of gene therapy.

Non-limiting examples of prokaryotic vector plasmids include: Arabinose-Regulated Promoter (Invitrogen pBAD Vector), T7 Expression Systems (Novagen, Promega, Stratagene): The pET-based vectors utilize the T7 RNA polymerase-based expression vectors, Trc/Tac Promoter Systems (Clontech (Palo Alto, Calif., U.S.A.), Invitrogen, Kodak, Life Technologies, MBI Fermentas (Lithuania), New England BioLabs, Pharmacia Biotech, Promega): Trc promoters are IPTG-inducible hybrid promoters. PL Promoters (Invitrogen pLEX and pTrxFus Vectors): Phage T5 Promoter (QIAGEN): tetA Promoter (Biometra pASK75 Vector): Lambda PR Promoter (Pharmacia pRIT2T Vector). A number of eukaryotic vector plasmids are available and may be used in the invention. For example, yeast, among others, may be used as a host to produce recombinant soluble peptide of choice. Non-limiting examples of yeast hosts that can be used for expression include Saccharomyces cerevisiae, Schizosaccharomyces pombe, Pichia pastoris, Hansela polymorpha, Kluyveromyces lactis, and Yarrowia lipolytica. Constitutive gene expression by a yeast plasmid cassette is commonly mediated (in S. cerevisiae and S. pombe) by the promoters for genes to the glycolytic enzymes: glyceraldehyde-3-phosphate dehydrogenase (TDH3), triose phosphate isomerase (TPI1), or phosphoglycerate isomerase (PGK1). Protein expression can also be regulated (induced) using the alcohol dehydrogenase isozyme II (ADH2) gene promoter (glucose-repressed), glucocorticoid responsive elements (GREs, induced with deoxycorticosterone), GAL1 and GAL10 promoters (to control galactose utilization pathway enzymes, which are glucose-repressed and galactose-induced), the metallothionein promoter from the CUP1 gene (induced by copper sulfate), and the PHO5 promoter (induced by phosphate limitation). Some of the commercially available yeast expression systems include: pYES2 (tightly regulated GAL1 promoter for galactose-induced expression and the URA3 gene for complementation selection in the S. cerevisiae INVSc1 strain (ura3-52) (Invitrogen); The Easy Select Pichia Expression Kit includes vectors (pPICZ series), P. pastoris strains (Clontech Laboratories) including the YEXpress™ Yeast Expression System, which features the pYEX 4T family of vectors for S. cerevisiae hosts; ESP™ Yeast Protein Expression and Purification Systems for S. pombe and pESC vectors for S. cerevisiae hosts. The pESC vector system allows for the coexpression of two different proteins (under control of the GAL1 and GAL10 promoters) from the same construct (Stratagene).

Other suitable eukaryotic expression systems include that of insect cells. Eukaryotic expression systems employing insect cell hosts are based upon one of two vector types: plasmid or plasmid-virion hybrids. The typical insect host is the common fruit fly, Drosophila melanogaster, encountered in practically any classical genetics laboratory. Other insect hosts include mosquito (Aedes albopictus), fall army worm (Spodoptera frugiperda), cabbage looper (Trichoplusia ni), salt marsh caterpillar (Estigmene acrea) and silkworm (Bombyx mori). In most all cases, heterologous protein Plasmid-based vector systems provide a mechanism for both transient and long-term expression of recombinant protein. This expression system is exemplified by the Drosophila Expression System (DES) available from Invitrogen. Novagen's S-protein-FITC staining of Sf9 Cells expressing SoTag™ fusion proteins using BacVector™ Novagen's pIE vectors are based on the baculovirus immediate early promoter ie-1. The plasmid-virion system is based upon the large, double stranded DNA baculovirus. The Autographica californica (alfalfa looper) nuclear polyhedrosis virus (AcNPV) virion is the most common source of the “expression cassette” for this system. The variety of commercially available insect-baculovirus expression systems, (all proven in the research arena), makes it a very difficult task not to select one (as opposed to developing a system from “scratch”) for expression of a given protein. Some of the commercially available insect expression systems are listed below. BacPAK Baculovirus Expression System; The pBacPAK1, 2, 3 series of transfer vectors offer cloning in all three reading frames under the AcNPV polyhedrin promoter; Coexpression of two proteins from the same expression cassette under the polyhedrin and p10 promoters is also possible with the pAcUW31 vector; In addition to the transfer vectors, the system also includes pBacPAK6 Viral DNA for generation of target gene carrying recombinant virus (Clontech). The baculovirus expression system marketed by Invitrogen, MaxBac, provides transfer vectors for the generation of single-gene expression cassettes. Three different versions of the single-gene vectors are available to enable insertion of the heterologous protein gene into the correct reading frame. In addition, a unique secretory peptide (honeybee melittin) gene is available in the pMelBac vector. Host cells for the MaxBac system include Sf9 and Sf21 strains and cabbage looper (T. ni) cell lines. Life Technologies' principal insect-baculovirus product is the BAC-TO-BAC Baculovirus Expression System. This system is unique in that the generation of recombinant baculovirus relies on site-specific transposition (between transfer and expression vector) in E. coli, as opposed to homologous recombination in insect host cells. The basis of this system is the pFASTBAC transfer vector, which contains the AcNPV polyhedrin promoter, with (pFASTBAC HT) or without a polyhistidine encoding gene, and the bacmid-containing E. coli host, DH10Bac. A dual-promoter transfer vector, pFASTBAC Dual, is also available for coexpression of two heterologous proteins, each under the control of the polyhedrin and p10 promoters. Novagen's BacVector System from is one of the most comprehensive and versatile systems available, providing over 30 different transfer vectors (pBac) and 3 different baculovirus expression vectors (BacVector). Many baculovirus expression vectors have a deleted polyhedron gene, but Novagen has gone one step further. The BacVector-2000 lacks polyhedron and several additional non-essential genes. The BacVector-3000 is similar to the BacVector-2000, but also lacks protease and chitinase genes that reduce degradation of expressed proteins and decrease cell lysis. Novagen's transfer vectors include positive screening with the selective marker, gus, and genes as N- and C-terminal peptide tags, such as cellulose binding domain (CBD), polyhistidine (HIS6; 6xHIS; His⁶) [SEQ ID NO:25], S-Tag™, to facilitate identification and purification, and secretory leader peptide (gp64 secretory leader) to direct extracellular export of the expressed protein product. There is also a choice of early, early/late, or very late (polyhedrin, p10, or pg64) promoters in the transfer vectors.

In some cases, mammalian expression systems may be used. Non-limiting examples of commercially available vectors for gene transfer and expression in mammalian cells are listed below. Products from Amersham Pharmacia (Sweden) include: pSVK3 (promoter SV40 Early), PSVL (promoter SV40 Late), pMSG (promoter MMTV-LTR (mouse mammary tumor virus), PCH110 (promoter SV40 Early); products from Promega include: pTarget T (promoter hCMV-IE^{b} (cytomegalovirus immediate early), pSI (promoter SV40 Early), pCI (promoter hCMV); products from Stratagene include: pOPRSVI (promoter RSV-LTR), pBK-CMV (promoter hCMV), pBK-RSV (promoter RSV-LTR), pDual (promoter hCMV (mutated), pCMV-Tag series (promoter hCMV); products from Sigma include: pFLAG-Tag series (promoter hCMV); products from Life Technologies include: pTet-Splice (promoter Tet); products from Clontech include: pTRE (promoter hCMV*-1), pRev-TRE (promoter hCMV*-1) pLNCX (promoter hCMV-IE), pLXS (promoter 5′LTR), pLXI (promoter 5′LTR), pSIR (promoter 5′LTR), pLAPSN (promoter 5′LTR), pIRES-bleo/hyg/neo/puro (promoter hCMV-IE), pIRES-EGFP (promoter hCMV-IE); products from Invitrogen include: pCDM8 (promoter hCMV), pcDNA1.1, pcdDNA1.1/Amp (promoter hCMV), pcDNA3.1-neo/zeo/hyg (promoter hCMV), pcDNA3.1/His/Myc-His/V5-His (promoter hCMV), pRc/CMV2 (promoter hCMV), pRc/RSV (promoter RSV-LTR), pSecTag2 (promoter hCMV), pDisplay (promoter hCMV), pZeoSV2 (promoter SV40), pREP series (promoter RSV-LTR), pCEP4 (promoter hCMV-IE), pEBVhis (promoter RSV-LTR), pcDNA4/HisMax (promoter hCMV), pVP22/myc-His (promoter HCMV), pIND series (promoterHSP (Heat Shock Ekcdysone protein)), pSin series (promoter SG (sindbis sub genomic promoter)), pEF series (promoter hEF-1 ((Elongation factor 1), pCMV series (promoter hCMV), pTracer series (promoter hCMV-IE, SV40, hEF-1(a)), pCMV-LIC (promoter hCMV-IE); products from PharMingen (San Diego, Calif., U.S.A.) include: pBacMam-1 (promoter Hybrid: hCMV-IE/avian actin); and products from Novagen include: pPOP (promoter mPGK/lacO phosphoglycerate kinase).

In some cases, it is desirable to express a large quantity of recombinant proteins or peptides using either a prokaryotic or eukaryotic expression system. For example, one may contemplate that a large quantity of recombinant GPCR proteins be expressed, harvested, purified, and used for a number of applications. However, like many large membrane-spanning proteins, recombinant GPCR proteins are difficult to express in large quantities, and often insoluble. One reason for the technical limitation may be that membrane proteins such as ion channels and ligand receptors are generally expressed at a relatively low concentration in a native cell, and cells may not efficiently process high levels of production (expression and/or intracellular transport to a correct subcellular domain). In particular, prokaryotic hosts, such as E. coli, lack eukaryotic chaperones, which may result in the mis-folding of expressed polypeptides that are derived from eukaryotic sources, such as many GPCR proteins that are of interest in a clinical context. Moreover, mature GPCR proteins in their native cellular environment are highly processed with post-translational modifications, and the availability of appropriate cellular biosynthesis machinery to carry out these modifications (e.g., enzymes responsible for each of the modification steps) may be limited in a host cell. Furthermore, at least in some cases mis-folded polypeptides are recognized and marked for degradation and never make it to the cell surface. Indeed, recombinant GPCR polypeptides expressed in E. coli are often found in an aggregate of inclusion bodies. Although the expressed polypeptides may be biochemically fractionated with the use of harsh detergents and isolated from the aggregates, such polypeptides are often biologically inactive.

To enhance expression and/or yield of a soluble GPCR of the invention, it is in some cases useful to choose a host cell system engineered to express altered levels of one or more enzymes that catalyze post-translational modifications of recombinant proteins or chaperons that may aid the folding of recombinant proteins. For example; E. coli that lacks one or more reductases may be used to prevent disulfide bonds from getting reduced. More specifically, the constructs described in the invention include short intra-domain linkers and disulfide bonds connecting secondary structures elements for the formation of stable folding into a native conformation. A limitation of the production of correctly folded proteins in E. coli has been the relatively high reducing potential of the cytoplasmic compartment; disulfide bonds are usually formed only upon export into the periplasmic space. To address this issue, bacterial strains with glutathione reductase (gor) and/or thioredoxin reductase (trxB) mutations may be used to enhance the formation of disulfide bonds in the E. coli cytoplasm. In addition, the Rosetta™ strains are designed to enhance the expression of eukaryotic proteins that contain codons rarely used in E. coli. For expression of soluble GPCR polypeptides, such as the exCCR5 chimera, Rosetta-gami™ cells (Novagen;) may be used. These conditions have the combined advantages of slowing down transcription and translation rates, as well as reducing the strength of the endothermic hydrophobic interactions that contribute to protein mis-folding.

Similarly, host cells may be engineered to express additional enzymes such as lipid transferases and glycosylases to enhance appropriate modifications of recombinantly expressed proteins. In addition, culture conditions may be altered to optimize yield; for example, growing E. coli at a lower temperature, e.g., 25-35° C., may promote the recombinant protein to fold correctly, thus improving the overall yield.

To prevent the formation of inclusion body, there are several ways to prevent aggregation of a protein in vivo. A traditional approach involves a reduced synthesis rate of the target gene product, in order to increase the chance of folding into a native state before it has the chance of aggregating with other folding intermediates. This can for example be achieved by using a vector with a low copy number, a weaker promoter or a weaker ribosome binding site (Galloway et al., 2003). Controlled transcription can also be accomplished with a low concentration of inducer (Baneyx et al., 2004). An alternative strategy is to decrease the temperature at which the recombinant protein is expressed (Schein, 1988; Chalmers et al., 1990; Strandberg et al., 1991). The use of low expression temperatures has the combined advantages of slowing down transcription and translation rates, as well as reducing the strength of the endothermic hydrophobic interactions that contribute to protein misfolding (Cleland, 1993). The presence of a small number of rare codons could potentially slow translation but does not affect the rate of protein synthesis very much in practice (Fahnert et al., 2004). The presence of multiple consecutive rare codons situated near the N-terminus of a coding gene sequence, may provide some beneficial effects on the protein expression and this has been observed (Imamura et al., 1999). Changes in the fermentation media composition, e.g. addition of non-metabolisable sugars such as sucrose and raffinose, can also affect inclusion body formation (Georgiou et al., 1986).

In E. coli only 10-20% of the host proteins receive help by chaperones for their folding and thus the amount of available natural chaperones is limited (Ewalt et al., 1997). Accordingly, a reduced inclusion body formation may be obtained by co-expression of chaperones and foldases (Hockney, 1994; Wall et al., 1995). Aggregation is most prominent for heterologous proteins containing disulfide bonds in their native state. Stable disulphide bonds do not form in the cytoplasm of E. coli, owing to the reducing redox conditions provided by the combined action of thioredoxins and glutaredoxins systems. Identification of the members of these systems and subsequent elucidation of their roles in disulfide bond reduction, has made it possible to manipulate the E. coli cytoplasm in some strains to be less reducing and thus more suitable for production of oxidized proteins (Derman et al., 1993; Baneyx et al., 2004). If optimization of the expression conditions is unfruitful, another strategy is to try to alter the protein of interest itself. Protein engineering using strategies like molecular evolution (Mosavi et al., 2003; Ito et al., 2004) or rational protein design (Mitraki et al., 1992; Murby et al., 1995) can be used to enhance the solubility of a protein. Fusion to a protein that has high internal solubility can also improve the solubility. Several proteins have been used for this purpose with varying success e.g. NU.S.A. (Davis et al., 1999), MBP and Theoredoxin (Sachdev et al., 1998). The fusion tag is thought to rapidly fold to a soluble domain, which may accelerate or stabilise the folding of the rest of the molecule, thereby acting as an intramolecular chaperone (Fedorov et al., 1997).

The production of purified GPCR proteins is important for research, diagnostic and therapeutic applications. Several research applications including antibody generation, i.e., for immunocytochemistry and immunoprecipitation studies, in vitro mapping of protein-protein, protein-DNA or protein-RNA interactions and structure determination are useful for studying biological processes. Other in vitro uses include: (1) mutational analysis to identify receptor elements involved in ligand recognition, (2) biochemical procedures for the affinity purification, detection, and analysis of ligands, (3) inhibition of receptor activity using biochemical or biological approaches, (4) screening of drug candidates that potentially inhibit receptor binding and (5) development of new biosensors. Other research based methods include methods of screening for GPCR receptor-specific (or ligand specific) molecules, that bind to a soluble GPCR polypeptide. The soluble ECL structures may also form the basis for the design of specific proteins inhibitors that will also have potential in the therapy of a wide range of diseases.

Cytokine receptors belong to families of receptor proteins, each with a distinctive structure and consist from 4 large families. There is a large family of cytokine receptors, which are divided into two subsets on the basis of the presence or absence of particular sequence motifs. Many cytokine receptors are members of the hematopoietin-receptor family, also called the class I cytokine receptor family. A smaller number of receptors fall into the class II cytokine receptor superfamily; many of these are receptors for interferons or interferon-like cytokines. Other super-families of cytokine receptors are the tumor necrosis factor-receptor (TNFR) family; and, the chemokine-receptor family, which are part of a very large family of large G protein-coupled receptors.

Naturally occurring soluble cytokine receptors regulate inflammatory and immune events by functioning as agonists or antagonists of cytokine signaling. Soluble receptors generally comprise the extracellular portions of membrane-bound receptors and therefore retain the ability to bind ligand. Soluble cytokine receptors cytokine receptors (class I, class II and TNF-receptor families are one trans-membrane spanning or GPI anchored proteins, unlike chemokine receptors, which are GPCRs) can be generated by several mechanisms, which include proteolytic cleavage of receptor ectodomains, alternative splicing of mRNA transcripts, transcription of distinct genes that encode soluble cytokine-binding proteins, release of full length receptors within the context of exosome-like vesicles, and cleavage of GPI-anchored receptors.

Soluble cytokine receptors, which either attenuate or promote cytokine signaling, are important regulators of inflammation and immunity. The key role that soluble cytokine receptors play in preventing excessive inflammatory responses. Recently, soluble receptors have been introduced into clinical medicine as a novel form of therapy.

Chemokine receptors are seven-transmembrane domain proteins that, in contrast to other cytokine receptor (family I, family II, TNFR families) cannot be easily engineered as soluble chemokine inhibitors. The work described herein indicates that soluble chemokine receptors can bind chemokines with high affinity, block the interaction of chemokines with their cellular receptors and predicts that chemokine-induced elevation of intracellular calcium levels and cell migration will be blocked. Soluble CCR5 thus represent a soluble inhibitor that binds and sequesters chemokines.

Thus, the present invention describes a new approach for construction of soluble receptors generally comprise the extracellular portions of 7 trans-membrane GPCR receptors and retain the ability to bind ligands (chemokines) and possible can be used in therapy like another members of soluble Naturally occurring cytokine receptors.

The invention thus contemplates that the soluble GPCRs could be used in high throughput screens for the identification of ligands that may have therapeutic application as (for example) anti-HIV, anti-inflammatory and anti-metastatic reagents. For instance, the soluble extracellular domain analogs of CCR5 and CXCR4 are used to carry out high throughput assays for screening new drugs that function as novel HIV entry inhibitors.

According to one aspect of the invention, methods for identifying a molecule that binds to a GPCR protein having a native conformation are provided.

In some embodiments, a method for identifying a binding molecule involves screening a sample or samples containing test molecules using a soluble GPCR analog (i.e., a soluble GPCR polypeptide with folded into substantially native conformation) described herein. The terms “identifying a molecule”, “identifying a ligand”, “identification of ligands” refer to a step or steps involving detecting and discovering target molecule or molecules or a method therefor. In some cases, molecules that are tested or screened for (i.e., test molecule or test sample) are known molecules or known samples such that the screening determines functional activity (such as binding) exists. In other cases, test samples contain unknown or unidentified molecules or compounds, which, subsequent to positive hits are to be identified.

The term “screening” refers to a process of testing test samples by assaying for a specific biological or biochemical property of interest. A screening may involve the use of soluble GPCR polypeptide in a solution; alternatively, the GPCR may be immobilized, e.g., on a membrane, beads, and the like.

As used herein, “a sample” or “a test sample” means any compound or a mixture of compounds desired to be tested for biological function or activity of interest or anything that may contain such compound therein. In some cases, test samples may contain a drug candidate.

Molecules or compounds that are screened in a assay using a soluble GPCR polypeptides of the present invention may include molecules or compounds of various function, which include but are not limited to: an agonist, an antagonist, an inhibitor, a blocker, a co-factor, etc.

Similarly, these molecules may be any type of chemical or biochemical compounds, including but are not limited to: protein, lipids, nucleic acids, peptides, small molecules, biosimilars, any fragment thereof and any mixture thereof.

As used herein, “a small molecule” includes both naturally occurring small molecules and synthetic small molecules.

As used herein, “a biosimilar” is defined as a biopharmaceutical product, e.g., a drug with a protein as an active ingredient which is produced by genetically modified cell lines, having therapeutic equivalence as compared to original product but a small change in the manufacturing process results in an important impact on the efficacy and safety of a product.

As used herein, “a high throughput assay” or “a high throughput screen” refers to a highly parallel, partially or fully automated screening system designed to systematically process a large number of samples for specific biological activity of interest. It is sometimes also referred to as “a high throughout screening.” Generally, a high throughput screen uses robotics to simultaneously test thoU.S.A.nds of distinct compounds in functional and/or binding assays. Therefore, such screening is often used to look for drug candidates.

“Isolating a molecule” shall mean that the molecule of interest is substantially isolated or purified from other components. A molecule may be isolated spectrometrically or physically.

In some embodiments, the method of identifying a molecule of interest involves screening for a second factor or a co-factor that promotes binding between a soluble GPCR polypeptide and its ligand. For example, screening is carried out either in the presence or in the absence of the second factor, and differential binding to the GPCR is determined. In some cases, binding between GPCR and its binding partner is abolished in the presence or in the absence of the second factor. If it is determined that the binding between GPCR and its ligand is abolished unless the second factor is present, the second factor is a required factor for the binding. If, however, binding between GPCR and its ligand is abolished in the presence of a second factor, the second factor is a blocker or an inhibitor for the binding. However, in other cases, binding may be partially modulated either in the presence or in the absence of the factor. In these cases, the factor is a regulator of binding between the GPCR and its ligand.

According to some embodiments, the method of the invention involves identifying a molecule that binds to a soluble GPCR that contains one or more mutations that cause alteration of binding profile as compared to that of the wild type counterpart. In some circumstances, these mutation are naturally-occurring mutations. For example, in some embodiments, the invention describes methods of identifying a molecule that bind to a virally encoded GPCR protein that modulates host cell immune system. Certain viruses, such as Herpes virus and Pox virus, are known to encode GPCR and cytokine homologues that modulate the host immune system in their favor. This approach will allow screening for molecules or compounds that can bind them and selectively block or inhibit the action of mutated GPCR proteins. The availability of recombinant GPCR proteins is also important for biomedical applications such as small molecule drug discovery and the production of therapeutic proteins and vaccines, including biosimilars.

Using the soluble GPCRs of the invention, one can design fast screening of small molecule antagonists that are able to complete with chemokine for binding to their specific receptor. For instance, the compounds can be used for proteomic screening.

As described herein, sexdCCR5-6xHis in E. coli was expressed in soluble form. Using conformation dependent antibodies, physiological ligands and R5 HIV-1 envelopes we demonstrated that sexdCCR5 is stable, likely to be correctly folded and can perform many of the same interactions as the native CCR5 receptor.

Protein interactions that are important for disease processes are likely to form specific targets for therapeutics. The two-hybrid system has been very useful in the identification of such targets in high-throughput proteomic screens. In particular, the bacterial two-hybrid system can be used to screen for peptides that bind sexdCCR5 and sexdCXCR4, while small molecules could be screened once a specific peptide is identified. This latter approach can provide an alternative to standard high throughput ELISA assays. The BacterioMatch™ two-hybrid system is based on a methodology developed by Dove, Joung, and Hochschild of Harvard Medical School (Boston, Mass., U.S.A.)(Dove, S. L. & Hochschild, A., 1997; Dove, S. L. & Hochschild, A., 1998; U.S. Pat. No. 5,925,523).

Thus, the soluble forms of the CCR5 and other GPRCs will have application for high throughput screening for receptor-specific ligands that will have potential therapeutic application for a wide variety of diseases e.g., AIDS, multiple schlerosis, rheumatoid arthritis and schizophrenia. While chemokines and their receptors are excellent therapeutic targets and GPCRs are targeted by 50% of medicines that are marketed currently, GPCRs are generally difficult to identify antagonists for and to evaluate binding sites. Using soluble chemokine receptors (or GPCRs) the present invention describes methods for fast screening of small molecule antagonists that are able to complete with chemokine for binding to their specific receptor. One such example is shown in Example 11. It should be appreciated that this strategy could be applied to many other G protein-coupled receptors (GPCRs), with a variety of potential applications including (1) mutational analysis to identify receptor elements involved in ligand recognition, (2) biochemical procedures for the affinity purification, detection, and analysis of ligands, (3) inhibition of receptor activity using biochemical or biological approaches, (4) screening of drug candidates that potentially inhibit receptor binding and (5) development of new biosensors. This approach could therefore facilitate efforts to identify or develop new therapeutics with anti-HIV, anti-inflammatory and anti-metastatic activities.

In these situations it is essential to be able to reliably express the proteins in a heterologous system and purify them so that they possess the same folds and structure as they would in a natural in vivo state. Such methods will have potential application for treating a wide variety of diseases associated with GPCR function, e.g. AIDS, infectious diseases, multiple sclerosis, rheumatoid arthritis, schizophrenia and others.

In one example, the sexdCCR5 and sexdCXCR4, soluble forms of CCR5 and CXCR4 chemokine receptors, the major coreceptors for HIV can be used therapeutically to treat HIV by preventing HIV viral entry. CXCR4 and CCR5 are coreceptors for the entry of HIV-1 strains, and chemokine binding to these receptors potently blocks HIV-1 infection in vitro.

The multi-step process of HIV entry into CD4⁺ cells, can be divided into three steps: (i) the virus envelope glycoprotein (gp120) binds to the CD4 receptor; (ii) The gp120-CD4 complex interacts with a chemokine coreceptor (CCR5 or CXCR4) on target cells; and (iii) the transmembrane subunit gp41 changes conformation to form a six-helix bundle, resulting in the fusion of the viral membrane with that of the target cells. Differential co-expression of CD4 and co-receptors on cells is correlated with their susceptibility to viral infection. Naturally occurring polymorphisms of the CCR5 gene generated by single point mutations and deletions that result in loss of function or reduced expression also play a role in resistance to HIV-1 infection and progress of the disease.

The process of the HIV envelope glycoprotein binding to and inducing fusion with target cells presents many opportunities for intervention. Inhibitors that target these processes are divided into three categories: (1) CD4 attachment inhibitors; (2) coreceptor interaction inhibitors; and (3) fusion inhibitors that target the viral gp41. All steps in the fusion cascade are suitable targets for pharmacologic intervention. The CD4 and coreceptor binding sites on gp120 are highly conserved and are targets of neutralizing antibodies and other small molecule inhibitors. Both small molecule inhibitors and blocking antibodies directed at the coreceptors themselves have shown potent inhibitory activity. Gp41 fusion intermediates are also targets for inhibition by peptide mimetics (e.g., T20), small molecules and antibodies that bind these structures.

Based on the ability of soluble polypeptides of the invention that are constructed from an HIV co-receptor protein, e.g., CCR5 and CXCR4, the invention further includes compositions and methods of use for chimeric derivatives that include a soluble GPCR polypeptide. Thus, the invention provides soluble forms of multi-domain chimeras comprised of HIV-reactive domains, that comprise a portion or portions of GPCR and factors that mediate viral entry into target cells, thereby each domain cooperatively inhibit (interfere with) viral interaction with and subsequent entry into a target cell. The term as used herein “chimera” or “chimeric” refers to a construct of a polypeptide or a corresponding nucleic acid encoding such a polypeptide that is derived from multiple domains or fragments of more than one sources such that the multi-domain structure is engineered to form a contiguous molecule. As used herein, “an HIV-reactive domain” refers to a domain or portion thereof derived from a factor (e.g., protein) that mediates or promotes the interaction between an HIV virion and host cell target, as to cause HIV entry and thus an HIV infection. For example. an HIV-reactive domain may be present in a viral factor that recognizes a target cell-surface receptor, a viral or host membrane protein that regulates membrane fusion, or any other co-factors that participate in the process of viral entry.

Accordingly, the present invention contemplates designing and constructing soluble HIV co-receptor analogs. More specifically, an HIV co-receptor analog is a chimeric derivative comprising a soluble HIV co-receptor polypeptide, further comprising fragments of critical regions of HIV Envelope glycoproteins involved in the viral entry process. Some embodiments of a chimeric derivative of a soluble HIV co-receptor analogs, and the rationale behind the strategy for designing an improved HIV inhibitor are described below.

The HIV envelope glycoproteins gp120 and gp41 are both encoded by the HIV env gene. Gp120 is on the surface of the viral envelope and is associated with the transmembrane gp41. Each envelope spike is arranged as a trimer with three gp120s and three gp41s. Gp120 comprises five conserved (C1-C5) and five variable (V1-V5) regions. The three-dimensional functional structure of gp120 has been characterized and shown to contain several intramolecular disulphide bonds (Hoxie, J. A., 1991; Leonard, C. K., et al., 1990), which are critical to maintain the conformational structure required for interaction with the CD4 and coreceptors. In its native form, gp120 comprises inner and outer domains. Determinants for CD4 binding are located mainly in the outer domain, while beta strand sections that comprise the coreceptor binding site are spatially separate. CD4 binding to gp120 induces conformational changes that involve (1) pulling together of inner and outer gp120 domains, (2) association of two β-sheet segments to form the bridging sheet that comprises the conserved part of the coreceptor-binding site, and (3) movement of the V1/V2 loops to uncover the coreceptor-binding site. Thus, CD4 binding locks gp120 into an optimal structure for coreceptor binding and induction of gp41 conformational changes required for fusion and entry into cells. Gp41 has four regions of functional importance. The first is the trans-membrane spanning region to anchor the protein into the viral membrane; next there are 2 external regions of helical structure (outside the membrane) called heptad repeats (HR1 and HR2) that interact together in the final stages of fusion to form a six-helix bundle or hairpin structure. Finally there is the fusion peptide region capable of piercing the CD4 cell membrane. When the gp120/CD4 complex interacts with the coreceptor, conformation changes in gp41 are induced that result in fusion. These changes include the extension of gp41 and insertion of the fusion domain into the cell membrane, refolding of gp41 into the hairpin, 6-helix bundle conformation that brings the transmembrane region (in virus membrane) and fusion peptide (in target membrane) into close proximity and induces fusion. These events are described in more detail below.

Attachment. Gp120 binds with a CD4 receptor on the surface of cell permissive to HIV infection e.g. T-helper cells and macrophages. The CD4 receptor binds between the inner and outer domains of HIV gp120. Its binding creates a cavity that is well-protected and conserved among different HIV strains. Electrostatic forces are involved in CD4-gp120 binding, with a positively charged ridge on the outermost domain of CD4 attracted to negatively charged amino acids on gp120. Van der Waals' forces and hydrogen bonds then help to stabilize the CD4-gp120 interaction. A phenylalanine at residue 43 on CD4 (Phe-43) is the only residue that binds to the cavity which has been called the Phe-43 cavity. This residue is quite significant in CD4-gp120 binding because it is estimated that it alone accounts for 23% of the total energy of CD4-gp120 binding (Madani, N., et al., 2004; Kwong, P. D., et al., 1998). Following CD4-gp120 binding, the gp120 conserved core undergoes conformational changes, moving from a flexible to a rigid state, allowing a subsequent interaction with the chemokine co-receptors (Myszka, D. G., et al., 2000). The Phe-43 cavity in HIV gp120 was initially pursued as a potential target for small molecules that could fill it and block the HIV entry (Kwong, P. D., et al., 1998; Kwong, P. D., et al., 2000; Wyatt, R., et al., 1998). However, small molecule drugs in development that block gp120 binding to CD4, bind to another site and may stabilize the unliganded form of gp120.

CD4 gp120 binding inhibitors and their mechanism of action. There are many molecules able to inhibit gp120-CD4 binding. They have different structures and mechanisms of action. PRO-542 (CD4-IgG2) is a recombinant antibody-based molecule that contains four copies of the CD4 domains that bind gp120 (Allaway, G. P. et al., 1995). TNX-355 is a monoclonal antibody directed against the CD4 receptor, which potently inhibits HIV entry. TNX-355 does not bind to the same CD4 site as gp120 and inhibits by blocking gp120 conformational changes which follow after CD4 is bound. CADA is a specific inhibitor of the CD4-gp120 binding that does not interact directly with the CD4 receptor or with gp120. CADA antiviral activity is probably due to its ability to down-regulate CD4 expression at a post-translational level (Vermeire, K. et al., 2003; Vermeire, K. et al., 2002). BMS-378806 binds with high affinity to HIV gp120, blocking binding to CD4 as well as the conformational changes in gp120 that occur after CD4 binding (Madani, N. et al., 2004; Lin, P. F. et al., 2003; Guo, Q. et al., 2003). The binding of BMS-378806 to HIV gp120 is co-receptor independent.

Resistance to CD4-gp120 inhibitors. Since the molecules that inhibit CD4-gp120 binding act in different ways, it is expected that resistance will also develop by different mechanisms. Cross resistance among these inhibitors is therefore expected to be minimal. In vitro derived viruses that are resistant BMS-378806 carry gp120 amino acid substitutions that line a deep, hydrophobic channel of unliganded gp120 are believed to stabilize the unliganded form of gp120 (Chen et al., 2005).

Coreceptor binding. Following attachment of gp120 to CD4, the coreceptor-binding site on gp120 is formed and exposed. The coreceptor binding site consists of the conserved beta strands of the bridging sheet and determinants on the V3 loop. Coreceptors are usually either CCR5 or CXCR4 chemokine receptors on the cell surface. Some virus strains use CCR5 or CXCR4 (monotropic), other strains can use both receptors (dual-tropic). Viruses that exploit CXCR4 emerge late on in the infection and are generally associated with more rapid disease progression. At least twelve different chemokine receptors can function as HIV coreceptors in cultured cells, but only CCR5 and CXCR4 are known at this time to play a role in vivo. CCR5 and CXCR4 belong to the seven transmembrane G protein-coupled receptor family. They are composed of four intracellular domains, seven transmembrane domains, three extracellular loops and one N-terminal extracellular domain. The CD4-gp120 complex binds to the coreceptor. V3 loop amino acids on gp120 determine whether CCR5 and/or CXCR4 is used. Accordingly, HIV isolates are classified as R5, X4 and R5/X4 strains, depending on their co-receptor use (Berger, E. A. et al., 1998). Coreceptor sites that bind gp120 have been mapped. For R5 viruses, the N-terminal domain and the second extracellular loop (ECL2) of CCR5 are essential for gp120 recognition, whereas for X4 strains, ECL2 is more critical (Picard, L. et al., 1997).

Resistance to CCR5 and CXCR4 antagonists. Two main resistance pathways are theoretically possible for CCR5 and CXCR4 antagonists. The first is a shift in co-receptor U.S.A.ge and the second results from changes in the HIV envelope which allow interaction between gp120 and co-receptor despite the presence of the inhibitor. Data available so far suggest that most CCR5 antagonist-resistant strains continue to use CCR5 rather than shifting to CXCR4. Furthermore, multiple mutations within different regions of HIV gp120 (V3, C2, V2, C4) account for the drug-resistant phenotype (Trkola, A. et al., 2002; Kuhmann, S. E. et al., 2004; Marozsan, A. J. et al., 2005). Most resistance mutations are specific for each of the different compounds, which may limit cross-resistance to other CCR5 antagonists. However, large clinical studies are needed to prove this concept. Preliminary findings with HIV isolates resistant to the CCR5 antagonist, maraviroc, have demonstrated that they remain susceptible to other CCR5 antagonists e.g. vicriviroc (Don, P. et al., 2005). In any case, CCR5 antagonist-resistant strains remain sensitive to other entry inhibitors, such as CD4-gp120 binding inhibitors and enfuvirtide. Resistance to CXCR4 antagonists is less well documented. However, mutations in the HIV gp120 V3 domain seem to contribute for the loss of susceptibility to these compounds, while mutations in other HIV gp120 regions may also contribute (de Vreese, K. et al., 1996; Schols, D. et al., 1998).

Fusion. The binding of gp120 to CD4 and CCR5 or CXCR4 likely induces the extension of gp41 and the insertion of it's N-terminal fusion peptide into the cellular membrane. Further conformational alterations result in the generation of a six-helix bundle, hairpin structure, formed from the HR1 and HR2 regions in gp41. The transition to this structure pulls the viral and cellular membranes together and promotes fusion of viral and the cellular membranes. Fusion leads eventually to the formation of a pore wide enough for the viral capsid to enter the cytoplasm.

Fusion inhibitors and their mechanism of action. Peptides based on the amino acid sequences of HR1 and HR2 of gp41 were originally recognized as inhibitors of HIV infection in the early 1990s (Wild, C. et al., 1992; Wild, C. T. et al., 1994). DP106, which mimicked a fragment of the HR1 amino acid sequence, was the first HIV peptide inhibitor described (Wild, C. et al., 1992). In 1993, the in vitro potency of another peptide, DP-108, based on the amino acid sequence of HR2, was demonstrated (Wild, C., T. Greenwell & T. Matthew, 1993). This molecule is currently known as T-20 or enfuvirtide. Enfuvirtide is derived from 36 amino acids of the HR2 region. Enfuvirtide binds to the HR1 region of gp41 and thus blocks the formation of the six-helix bundle structure, which is critical for the fusion process. Enfuvirtide was approved for the treatment of HIV infection in 2003 (Robertson, D., 2003). T-1249 represents a second generation of fusion inhibitors. This molecule is a 39 amino acid peptide based on an HR2 sequence that overlaps the enfuvirtide sequence (Kilby, J. M. & J. J. Eron, 2003). Interestingly, T-1249 was active against HIV-1 enfuvirtide-resistant strains as well as against HIV-2 and SIV (Lalezari, J. P. et al., 2005).

Resistance to fusion inhibitors. Clinical studies have shown that resistance in patients receiving enfuvirtide is conferred by mutations in the HR1 region of gp41 leading to amino acid substitutions in HR1 codons 36 to 45 (Wei, X. et al., 2002; Sista, P. R. et al., 2004; Poveda, E. et al., 2004). A spectrum of different resistance mutations has been described in this region, each reducing susceptibility to enfuvirtide (Poveda, E. et al., 2005).

Overall, enfuvirtide should be considered as a drug with a low genetic barrier for resistance. A wide range of susceptibility to enfuvirtide for viral isolates has been shown with resistant viruses also occurring in some untreated individuals (Poveda, E. et al., 2005; Sista, P. R. et al., 2004). Host determinants (e.g. the level of co-receptor expression on target cells) may also influence the susceptibility to enfuvirtide. In this way, the presence of high levels of CCR5 on the cellular surface might result in more rapid HIV fusion, reducing the time during which HIV gp41 could be targeted by enfuvirtide. Accordingly, heterozygous individuals carrying the delta 32 CCR5 polymorphism who express low levels of CCR5, respond more favorably to enfuvirtide (Reeves, J. D. et al., 2002).

Because the gp41 ectodomain initially interacts with the target cell surface through its highly hydrophobic N terminus, which is believed to insert into the target membrane, for the fusion event to take place the ectodomain is required to interact with its target. Accordingly, the invention contemplates targeting this step to prevent membrane fusion, thereby inhibiting HIV entry into the cell. The term, “gp41 ectodomain” refers to the portion of the gp41 protein that is localized external to the virion membrane (as opposed to an endodomain which is localized within the virion); the ectodomain is exposed to the outside of the viral surface and thus available for interaction with a target cell. The ectodomain of gp41 consists of ˜683 amino acid residues, which corresponds to the amino segment of the protein. Segments of the ectodomain believed to be involved in membrane fusion have been mapped to amino acid residues ˜512-683. This region comprises several structurally (and thus functionally) distinct domains, including hydrophobic domains, heptad repeat domains and loop region.

As used herein, a “C-terminal intramolecular interaction domain” is defined as a segment of the gp41 ectodomain spanning about 628-683 (this also include an epitope for HIV-1 neutralizing antibody 2F5 (MAb 2F5) which is localized at 661-684. This segment of the ectodomain encompasses two subdomains, namely, a heptad repeat termed the C-terminal heptad repeat (OAR) followed by a hydrophobic region called the tryprophan rich pre-transmembrane domain, which has been proposed to serve as an internal fusion peptide (IFP).

As used herein, an “N-terminal intramolecular interaction domain” is defined as a segment of the gp41 ectodomain spanning about 512-581. This segment of the ectodomain encompasses two subdomains, namely, the N-terminal hydrophobic fusion peptide (FP) region and an adjacent α-helical leucine/isoleucine zipper termed the N-terminal heptad repeat (NHR). The FP is believed to play a pivotal role in the fusion event by inserting into the target membrane and directly effecting the fusion of apposing bilayers.

CCR5 and CXCR4 antagonists are divided into three groups depending on their size. Large molecules, such as PRO-140 (a CCR5 specific monoclonal antibody), or molecules with a medium size e.g. Met-RANTES and AOP-RANTES (derivatives of RANTES, a natural CCR5 ligand) either block HIV binding directly or induce removal of CCR5 from the cell surface by internalization into endosomes. Several small-molecule inhibitors directed against CCR5 (TAK-779, SCH-C, SCH-D, UK-427857 and GW-873140) or CXCR4 (AMD3100 and KRH-1636) have been developed. These small molecule antagonists are believed to stabilize CCR5 in a conformation that HIV can't recognize. TAK-779 was the first non-peptide molecule that blocked in vitro replication of R5 strains by interfering in their interaction with the CCR5 coreceptor. The binding site is localized in a CCR5 transmembrane cavity formed by the 1, 2, 3 and 7 co-receptor transmembrane regions.

The sexdCCR5 and sexdCXCR4 constructs and derivatives thereof described in the invention are useful for mimicking the chemokine coreceptors (CCR5 or CXCR4) on target cells and thereby preventing viral entry. The fusion proteins, e.g., chimeric derivatives, include at least 2 extracellular domains as described above, and preferably more. In some cases, a chimeric construct of the instant invention may be comprised of one or more fragments derived from CCR5 combined with one or more fragments derived from CXCR4. The fusion proteins can be administered to a subject having HIV in an effective amount to treat HIV.

A subject having HIV is a human that is capable of being infected with a human immunodeficiency virus. A “subject” shall also mean or other vertebrate animals including a dog, cat, horse, cow, pig, sheep, goat, chicken, monkey, rat, mouse capable of being infected with an immunodeficiency virus.

As used herein, the term “treat” in reference to a disease or condition shall mean to intervene in such disease or condition so as to prevent or slow the development of, prevent, inhibit, or slow the progression of, halt the progression of, or eliminate the disease or condition. As used herein, the term “inhibit” shall mean reduce an outcome or effect compared to normal.

The compositions of the invention may be administered alone or in combination with other drugs for the treatment of HIV. Such drugs include the inhibitors and antagonists discussed above as well as other anti-viral therapies and therapeutics designed to treat symptoms or secondary conditions associated with AIDS. Anti-HIV medicaments include but are not limited to the following. Zidovudine (AZT), for treating HIV, is a nucleoside analogue. Lamivudine (2′,3′-dideoxy-3′-thiacytidine, 3TC) used for treatment of HIV is a reverse transcriptase inhibitor marketed by GlaxoSmithKline (United Kingdom) under the brand names Epivir® and Epivir-HBV®. It is also called 3TC. It is an analogue of cytidine. Abacavir (ABC) is a nucleoside analog reverse transcriptase inhibitor (NARTI) used to treat HIV and AIDS. It is available under the trade name Ziagen™ (GlaxoSmithKline) and the combination drugs Trizivir™ (abacavir, zidovudine and lamivudine) and Kivexa®/Epzicom™ (abacavir and lamivudine). ABC is an analog of guanosine (a purine). Its target is the viral reverse transcriptase enzyme. Didanosine (2′-3′-dideoxyinosine, ddI) is sold under the trade names Videx® and Videx EC®. It is a reverse transcriptase inhibitor, effective against HIV and used in combination with other antiretroviral drug therapy as part of highly active antiretroviral therapy (HAART). Didanosine (ddI) is a nucleoside analogue of adenosine having hypoxanthine attached to the sugar ring. Emtricitabine (FTC), with trade name Emtriva® (formerly Coviracil), is a nucleoside reverse transcriptase inhibitor (NRTI) for the treatment of HIV infection in adults. Emtricitabine is an analogue of cytidine. Enfuvirtide (INN) is an HIV fusion inhibitor, marketed under the trade name Fuzeon (Roche; Switzerland). Nevirapine, also marketed under the trade name Viramune® (Boehringer Ingelheim; Germany)), is a non-nucleoside reverse transcriptase inhibitor (NNRTI) used to treat HIV-1 infection and AIDS but is a protease inhibitor. Stavudine (2′-3′-didehydro-2′-3′-dideoxythymidine, d4T, brand name Zerit®) is a nucleoside analog reverse transcriptase inhibitor (NARTI) active against HIV. Stavudine is an analog of thymidine.

The fusion proteins of the invention and the anti-HIV therapy may be administered at the same time or in alternating cycles or any other therapeutically effective schedule. “Alternating cycles” as used herein, refers to the administration of the different active agents at different time points. The administration of the different active agents may overlap in time or may be temporally distinct. The cycles may encompass periods of time which are identical or which differ in length. For instance, the cycles may involve administration of the fusion proteins on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc, with the anti-HIV therapy being administered in between. Alternatively, the cycles may involve administration of the fusion proteins on a daily basis for the first week, followed by a monthly basis for several months, and then every three months after that, with the anti-HIV therapy being administered in between. Any particular combination would be covered by the cycle schedule as long as it is determined that the appropriate schedule involves administration on a certain day.

Because the instant invention embraces soluble GPCR polypeptides that retain substantially native conformation such that they effectively bind their corresponding ligands, the invention is useful for developing a variety of GPCR-based vaccines. These vaccines may be used to treat or prevent a number of diseases associated with impaired function of GPCR signaling.

For example, the connection between HIV and the chemokine system has implications for the development of an effective HIV vaccine. Current vaccine studies generally assume that a vaccine will induce both cellular immunity and neutralizing antibodies. The latter task is hampered by the fact that the only target for antibody induction is the envelope gene, which also displays the highest sequence diversity. The invention therefore contemplates that the GPCR such as sexdCCR5 and sexdCXCR4 plus soluble CD4 or CD4-sexdCCR5 chimera could serve as conformational framework to stabilize a specific neutralizing epitope in order to induce such an immunological response upon immunization. Thus, the GPCR constructs of the invention have utility in vaccines.

The invention also includes the use of an adjuvant for formulating vaccine in some aspects. The adjuvant in some embodiments is an adjuvant that creates a depo effect, an immune stimulating adjuvant, or an adjuvant that creates a depo effect and stimulates the immune system. Preferably the adjuvant that creates a depo effect is selected from the group consisting of alum (e.g., aluminum hydroxide, aluminum phosphate) emulsion based formulations including mineral oil, non-mineral oil, water-in-oil or oil-in-water emulsions, such as the Seppic ISA series of Montanide adjuvants; MF-59; and PROVAX. In some embodiments the immune stimulating adjuvant is selected from the group consisting of oligonucleotides containing unmethylated CpG dinucleotide motif, saponins purified from the bark of the Q. saponaria tree, such as QS21; poly[di(carboxylatophenoxy)phosphazene] (PCPP) derivatives of lipopolysaccharides such as monophosphorlyl lipid (MPL), muramyl dipeptide (MDP) and threonyl muramyl dipeptide (tMDP); OM-174; and Leishmania elongation factor. In one embodiment the adjuvant that creates a depo effect and stimulates the immune system is selected from the group consisting of ISCOMS; SB-AS2; SB-AS4; non-ionic block copolymers that form micelles such as CRL 1005; and Syntex Adjuvant Formulation. One or more of the adjuvants may be used in combination to augment the effect of vaccines.

Vaccines according to the invention are described herein as “antigenic” or “autoimmunogenic”, meaning that they elicit production of specific antibodies in an individual receiving the vaccine which antibodies recognize or bind to the antigen to which the vaccine is specific. Thus, the sexdGPCR vaccines of this invention are immunogenic moieties that have the capacity to stimulate the formation of antibodies which specifically bind GPCR and/or inhibit GPCR activity. The generation of an antibody response capable of neutralizing a broad range of clinical isolates remains an important goal of human immunodeficiency virus type 1 (HIV-1) vaccine development. Accordingly, the invention also describes herein the generation of soluble forms of chemokine receptors that will be helpful for the production and characterization of high affinity monoclonal or humanized antibodies to highly conserved epitopes on HIV-1 envelope glycoproteins, for example.

The vaccines described herein may be administered to a subject to elicit relatively high levels of antibodies that inhibit or reduce GPCR activity in the subject needing treatment or in another subject and then the antibodies may be administered to the subject needing treatment.

A vaccine described herein may comprise one or more copies of the same or different antigens. The one or more antigens may be attached to a common carrier molecule using linkages such as disulfide bonds or other linkages. Examples of common carrier molecules that may be used in vaccine compositions described herein include, without limitation, serum proteins (e.g., serum albumin), “core” molecules (e.g., multiple antigenic peptide (MAP) arrangements; see, e.g., Tam et al., Proc. Natl. Acad. Sci. U.S.A., 85: 5409-5413 (1988); Wang et al., Science, 254: 285-288 (1991); Marguerite et al., Mol. Immunol., 29: 793-800 (1992)), injectable resin particles, injectable polymeric particles, and the like, which have one or more functional groups available to form a bond with an antigen described herein. In addition, by using the appropriate linkages or linker molecules, different species of antigen may be attached to the same common carrier molecule.

The vaccines and/or optionally other therapeutic agents such as adjuvants may be administered simultaneously or sequentially. When the compounds are administered simultaneously they can be administered in the same or separate formulations, but are administered at the same time. The compounds are administered sequentially with one another, when the administration of the vaccine or antigen and other therapeutic agent is temporally separated. The separation in time between the administration of these compounds may be a matter of minutes or it may be longer. Other therapeutic agents include but are not limited to adjuvants.

Appropriate dosing for use of a vaccine composition described herein can be established using general vaccine methodologies of the art based on measuring parameters for which a particular vaccine composition is proposed to affect and the monitoring for potential contraindications. In addition, data available from studies of previously described vaccines may also be considered in the development of specific dosing parameters for the improved vaccine compositions described herein.

The vaccine compositions are administered in one or more doses over time, with an initial priming vaccination being followed, typically, by one or more “booster” vaccinations at a later time to raise or maintain an antibody titer. The exact dosing and boosting schedule will be determined by the practitioner to optimize the safety and effectiveness of the vaccine composition for modulating activity.

The GPCRs have a huge range of biologically important functions, in addition to being useful as vaccines. Malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders.

CCR5, expressed in lymphoid organs and cells, with multiple transcripts with 5′ end heterogeneity and dual promoter U.S.A.ge, mediate macrophage-tropic strains of HIV-1 entry in CD4+ cells with a reduced risk of AIDS lymphoma in patients with the CCR5-delta 32 mutation, G protein coupled receptor superfamily. As such, CCR5 plays an important role in mediating the inflammatory reaction of diseases such as rheumatoid arthritis and multiple sclerosis. The CC chemokine receptor CCR5 is a major coreceptor for the entry HIV-1 R5 viruses into cells.

CXCR4 is expressed in numerous tissues, such as peripheral blood leukocytes, spleen, thymus, spinal cord, heart, placenta, lung, liver, skeletal muscle, kidney, pancreas, cerebellum, cerebral cortex and medulla (in microglia as well as in astrocytes), brain microvascular, coronary artery and umbilical cord endothelial cells. CXCR4 is involved in haematopoiesis and in cardiac ventricular septum formation. It plays an essential role in vascularization of the gastrointestinal tract, probably by regulating vascular branching and/or remodeling processes involving endothelial cells. CXCR4 is also involved in cerebellar development. In the CNS, CXCR4 may mediate hippocampal neuron survival. CXCR4 acts as a coreceptor for HIV-1 X4. Defects in CXCR4 are a cause of WHIM syndrome; also called warts, hypogammaglobulinemia, infections, and myelokathexis. WHIM syndrome is an immunodeficiency disease characterized by neutropenia, hypogammaglobulinemia and extensive human papillomavirus (HPV) infection.

In addition to methods of treating HIV, many other therapeutic methods are encompassed by the invention. Because exCCR5 is able to bind three different ligands that depend on the conformational integrity of CCR5, namely, HIV-1 gp120, the chemokine RANTES and a CCR5-specific monoclonal antibody, the soluble ECL structures may also form the basis for the design of specific proteins inhibitors that will also have potential in the therapy of a wide range of diseases. Soluble cytokine receptors, which either attenuate or promote cytokine signaling, are important regulators of inflammation and immunity. For example, chemokines direct migration of immune cells into sites of inflammation and infection. Chemokine receptors are seven-transmembrane domain proteins that, in contrast to other cytokine receptors, cannot be easily engineered as soluble chemokine inhibitors. The work described herein indicates that soluble chemokine receptors can bind chemokines with high affinity, block the interaction of chemokines with their cellular receptors and predicts that chemokine-induced elevation of intracellular calcium levels and cell migration will be blocked. Soluble CCR5 thus represent a soluble inhibitor that binds and sequesters chemokines. This novel approach should provide new insights into disease pathogenesis and generate new therapeutic targets.

Although seven-transmembrane domain proteins, such as chemokine receptors, unlike other cytokine receptors, cannot be easily engineered as soluble chemokine inhibitors, the work presented herein indicates that soluble chemokine receptors of the instant invention can bind chemokines with high affinity, block the interaction of chemokines with their cellular receptors and predicts that chemokine-induced elevation of intracellular calcium levels and cell migration will be blocked. Soluble CCR5 thus represent a soluble inhibitor that binds and sequesters chemokines. This novel approach should provide new insights into disease pathogenesis and generate new therapeutic targets.

There are many GPCRs associated with diseases that may be treated using the compositions of the invention. Some non-limiting examples are provided below. Sometimes a disease results from a mutation in the receptor e.g. rhodopsin and night blindness. For others e.g. asthma, the underlying cause is not known, however treatment with CCR5 antagonists in an animal model of asthma, helps to alleviate the resulting inflammation. In addition to it's role in HIV entry, CCR5 plays an important role in mediating the inflammatory reaction of diseases. Chemokine receptor CCR5 is upregulated by pro-inflammatory cytokines and has been frequently associated with inflammatory and autoimmune diseases including; transplant, asthma, atherosclerosis, peripheral neuropathy, nephritis, IBD (inflammatory bowel disease), AIDS, Cancer, MS (multiple sclerosis); RA, (rheumatoid arthritis). Thus CCR5 fusion proteins of the invention are useful for treating disease such as HIV, asthma, RA and MS as well as the other listed diseases.

CXCR4 chemokine receptor is constitutively expressed and is involved in trafficking of cells in development. Diseases associated with CXCR4 chemokine receptor include AIDS, cancer, bone marrow transplantation. CXCR4 is also involved in haematopoiesis and in cardiac ventricular septum formation. It plays an essential role in vascularization of the gastrointestinal tract, probably by regulating vascular branching and/or remodeling processes involving endothelial cells. CXCR4 may also be involved in cerebellar development. In the CNS, CXCR4 may mediate hippocampal neuron survival.

Many other chemokine receptors have been associated with different disease either by the presence of polymorphisms (mutations) or changes in expression. CXCR1 and CXCR2 for instance, have been associated with sepsis. atherosclerosis, Psoriasis, rheumatoid arthritis, chronic obstructive pulmonary disease; CXCR3 has been associated with transplant, multiple sclerosis, psoriasis, rheumatoid arthritis; CCR1 has been associated with multiple sclerosis, rheumatoid arthritis, transplant, renal fibrosis, CCR4 has been associated with asthma, skin diseases; CCR6, CCR7 have been associated with asthma, CCR8, CCR9, CCR10 have been associated with inflammatory bowel disease, CX3CR1 has been associated with atherosclerosis. Thus, fusion proteins of the chemokine receptors are useful for treating the indicated diseases.

Family 1a receptors such as rhodopsin have been associated with Congenital night blindness and Retinitis pigmentosa.

Family 1b receptors, for instance, CCK2R (cholecystokinin-B/gastrin receptor subtype 2) has been associated with Gastric carcinoid tumors, KSHV-GPCR (Kaposi sarcoma-associated herpes virus) (open reading frame ORF 74), pirated by human herpesvirus 8, homologous to CXCR2 has been associated with Kaposi's sarcoma, primary effusion lymphoma, US28-GPCR Pirated by human cytomegalovirus, homologous to CC chemokine receptor CCR 1 has been associated with Atherosclerosis, infections in immunocompromised patients.

Family 1c receptors, for instance, TSHR, receptor for thyrotropin has been associated with Adenoma or hyperplasia associated with hyperthyroidism, LHR, receptor for luteinizing hormone has been associated with Male precocious puberty, Leydig cell tumor associated with male precocious puberty, Receptor for follicle-stimulating hormone has been associated with Normal semen parameters despite hypophysectomy.

Family 2 receptors, for instance, PTH-PTHrPR receptor for parathyroid hormone/parathyroid hormone-related peptide has been associated with ansen-type metaphyseal chondrodysplasia, dwarfism, hypercalcemia, hypophosphatemia.

Family 3 receptors, for instance, CaR, Calcium-sensing receptor has been associated with Autosomal dominant hypocalcemia.

Thus, fusion proteins of the Family 1, 2, 3 receptors are useful for treating the indicated diseases.

For any compound described herein a therapeutically effective amount can be initially determined from cell culture assays. In particular, the effective amount of fusion protein can be determined using in vitro stimulation assays. The stimulation index can be used to determine an effective amount of the particular fusion protein for the particular subject, and the dosage can be adjusted upwards or downwards to achieve the desired levels in the subject.

Therapeutically effective amounts can also be determined in animal studies. For instance, the effective amount of fusion protein and optionally other therapy to induce a therapeutic response can be assessed using in vivo assays such as assays of viral load when HIV is being treated. Relevant animal models include primates infected with simian immunodeficiency virus (SW). Generally, a range of CpG nucleic acid doses are administered to the animal along with a range of anti-HIV therapy doses. Reduction in viral load in the animals following the administration of the active agents is indicative of the ability to reduce the viral load and thus treat HIV infection.

The applied dose of both the fusion protein and optionally the other therapy can be adjusted based on the relative bioavailability and potency of the administered compounds. Adjusting the dose to achieve maximal efficacy based on the methods described above and other methods are well within the capabilities of the ordinarily skilled artisan. Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

Pharmaceutical compositions comprising a soluble GPCR polypeptide as described is included in the invention.

As used herein, a pharmaceutically-acceptable carrier means a non-toxic material that does not interfere with the effectiveness of the biological activity of the active ingredients, i.e., the ability of the agent to modulate killer T cell activity. Pharmaceutically acceptable carriers include diluents, fillers, salts, buffers, stabilizers, solubilizers and other materials which are well-known in the art. Exemplary pharmaceutically acceptable carriers for peptides are described in U.S. Pat. No. 5,211,657. The agents of the invention may be formulated into preparations in solid, semi-solid, liquid or gaseous forms such as tablets, capsules, powders, granules, ointments, solutions, depositories, inhalants and injections, for oral, parenteral or surgical administration. The invention also embraces pharmaceutical compositions which are formulated for local administration, such as by implants.

According to the methods of the invention the agents can be administered by injection, by gradual infusion over time or by any other medically acceptable mode. The administration may, for example, be intravenous, intraperitoneal, intramuscular, intracavity, subcutaneous or transdermal. Preparations for parenteral administration includes sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation. The methods of the invention also encompass administering biliary glycoprotein binding agents in conjunction with conventional therapies for treating immune system disorders. For example, the methods of the invention may be practiced simultaneously with conventional treatments. The particular conventional treatment depends, of course, on the nature of the disorder.

The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; and phosphoric acid in a salt.

The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular compound selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intrathecal, intramuscular, or infusion.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous preparation of the fusion proteins, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intrathecal, intramuscular, etc. administrations can be found in Remington's Pharmaceutical Sciences, Mack Publishing Co., Easton, Pa.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. Use of a long-term sustained release implant may be desirable. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The invention will be more fully understood by reference to the following examples. These examples, however, are merely intended to illustrate the embodiments of the invention and are not to be construed to limit the scope of the invention.

### EXAMPLES

### Example 1

### Design of Common Extracellular Domain Unit of Soluble CCR5 Chimera

In the present work, we have focused on the solvent-exposed extracellular loop segments of the CCR5 chemokine receptor. Like other members of group 1b GPCRs, CCR5 contains an N-terminus and 3 ECL domains. There are four cysteine residues that form two disulphide bonds. The previous biochemical and genetic studies demonstrated that CCR5 interacts with chemokines and HIV-1 through an exposed extracellular structural motif comprising part of the N-terminus and ECL1, ECL2 domains (Olson, W. C. et al., 1999; Blanpain, C. et al., 1999; Dragic, T. et al., 1998; Farzan, M. et al., 1998).

It was hypothesized that all extracellular domains of CCR5 could fold independently of the TM helices and would functionally mimic the extracellular surface of an intact CCR5 chemokine receptor by interacting with various protein targets. Accordingly, a soluble exCCR5 molecule to satisfy the following criteria was designed: (1) It should incorporate all extracellular domains of CCR5 (N-terminus [SEQ ID NO:27], ECL1 [SEQ ID NO:28], ECL2 [SEQ ID NO:29], and ECL3 [SEQ ID NO:30]); (2) these elements should be connected by short turn flexible linkers to allow the adoption of the most natural, ligand-binding conformation; (3) there should be two disulfide bonds as predicted for CCR5; (4) the relative orientation all of the extracellular receptor elements should be that predicted for the native receptor.

A major challenge in the present investigation was to define the lengths of the extracellular loops and TM helices of the native CCR5 molecule, so that a minimal construct could be designed where the ECL loops were sufficiently close to allow favorable interdomain interactions. The schematic representation of the design strategy of soluble extracellular based-domain GPCRs analog (e.g. for exCCR5) is shown in FIG. 1A. The amino acid alignment for full length human CCR5 [SEQ ID NO:26] and extracellular domain-based exCCR5 is shown in FIG. 1B.

### Example 2

### Design of Interdomain Linkers

The second (but equally important) consideration was the choice of linker sequence to be placed between the N-terminus and the extracellular loops (and a 6xHis tag) in order to afford some degree of conformational flexibility. Control of structural flexibility is essential for the proper functioning of a large number of proteins and multiprotein complexes. At the residue level, such flexibility occurs due to local relaxation of peptide bond angles whose cumulative effect may result in large changes in the secondary, tertiary or quaternary structures of protein molecules. Linkers are thought to control favorable and unfavorable interactions between adjacent domains by means of variable flexibility furnished by their primary sequence.

Chemokine receptors, including CCR5, share two additional cysteine residues (compared to other GPCR families) which are thought to form a disulfide bond between the N-terminus and the third extracellular loop resulting in their close proximity. A second disulfide bond links ECL1 and ECL2. These two disulfide bridges probably impose a structural constraint on extracellular receptor domains and thereby stabilize a receptor conformation which is capable of ligand binding. The selection or correct design of the short flexible, turn forming linker sequence is particularly important for stable folding, Cys-bridge formation and domain-domain interactions e.g. between ECL1-ECL2 loops. The linkers must also accommodate C101 in ECL1 and C178 in ECL2, which localize close to the TM alpha-helices. In our construct, the CCR5 N-terminus, ECL1 ECL2, and ECL3 domains, interact through the linker regions and two Cys bridges, thereby giving rise to a “double-hinged” type of domain movement. The domain movement will be restricted as a result of these double linkers but should form tighter links. Domain movements, in general, appear to be spread uniformly over the interdomain contact regions of exCCR5.

Taking into account these structural restrictions, we designed short (4 amino acid), flexible PGGS linkers [SEQ ID NO:1] that are likely to impose a tight turn, for the exCCR5 chimera. The inability of proline residues to donate hydrogen bonds or participate comfortably in any regular secondary structure conformation means they are usually involved in a tight turn. The polyglycine bridge folds back, allowing the electron donor to come into direct contact with the electron acceptor attached to the opposite end of molecule. We hypothesized that short flexible linker PGGS [SEQ ID NO:1] will be joined close enough to the ends of the neighbor ECL domains of CCR5 and will enable favorable interdomain contacts. We analyzed the position of the putative linker regions in the context of exCCR5 using MacVector multiple prediction program runs. According to these analyses, PGGS linker [SEQ ID NO:1] residues are shown to be relatively more flexible and unlikely to be exposed compared to the flanking ECL domains. According to the secondary structure prediction of Chou and Fasman, the PGGS linker residues as part of exCCR5, was shown to be like the Chou and Fasman ‘probable turn’ type. Also, we prepared a homology model of the exCCR5 protein. These predictive results indicated that the extracellular domains joined by PGGS [SEQ ID NO:1] short flexible turn linkers may bring all four CCR5 elements sufficiently close to allow simultaneous interaction with ligands.

### Example 3

### The Extracellular Domain-Based PCR Strategy to Generate a Fusion Protein from Four Extracellular Domains of exCCR5

The next step in the preparation of exCCR5 chimera was the design of a PCR strategy. The strategy described here can be used to generate a fusion protein from four extracellular domains of other members of GPCRs and is based on a rational design of long internal primers for ECL domains and linkers. Typically, the extracellular loops 1 and 3 (ECL-1 and ECL-3) are relatively short and may mainly connect transmembrane helices, while the N-terminal segment and ECL-2 are significantly longer. The design of amino acid sequences and lengths of the connecting linkers can vary but there are some important restrictions for linker engineering. The linkers must control the distance between domains, orientation, and relative motion of functional domains.

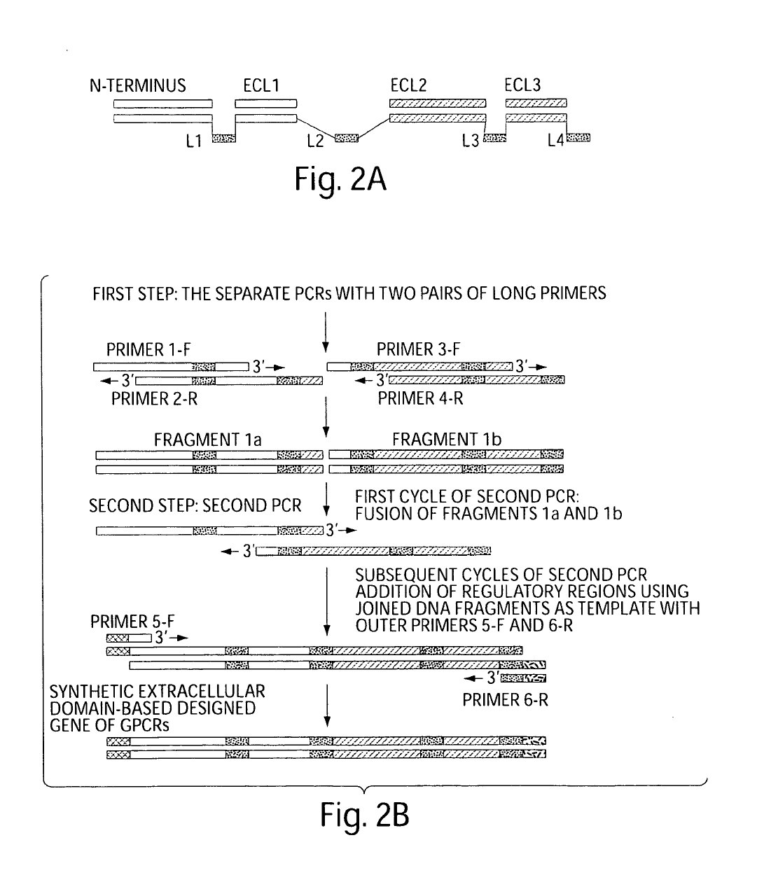

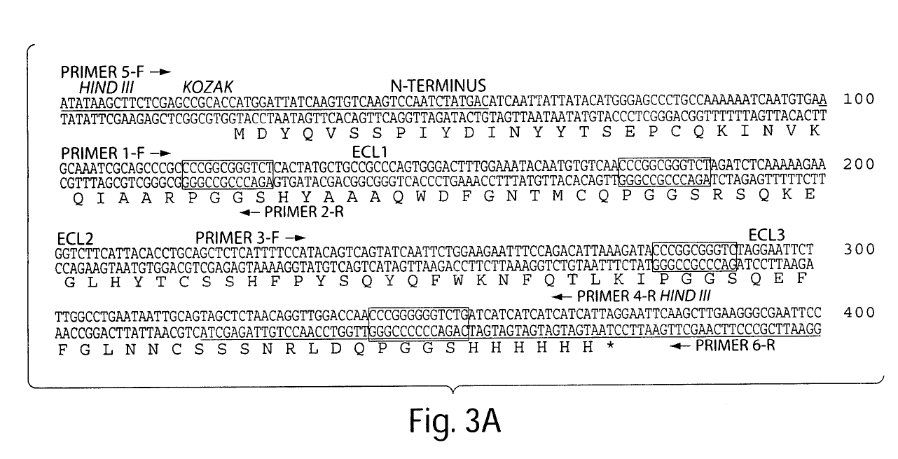

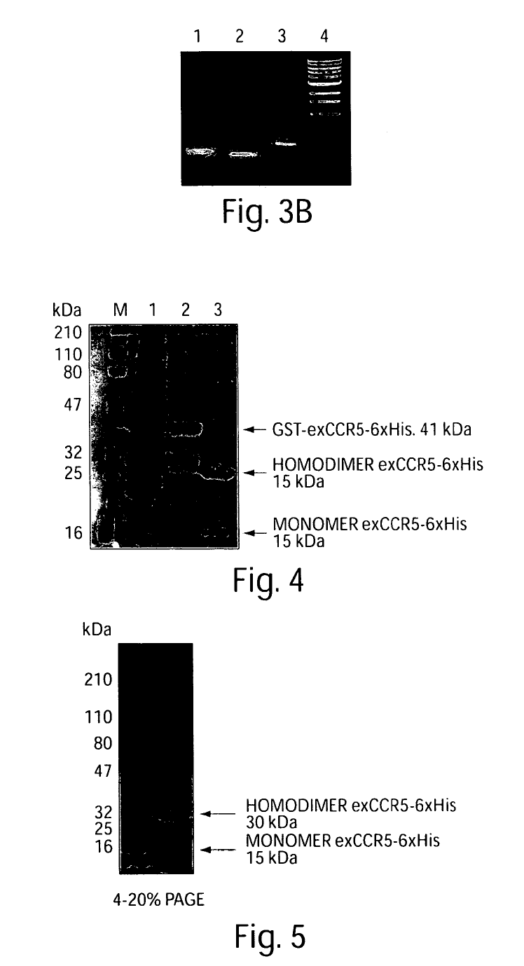

The technique joins the coding sequences of extracellular domains of chemokine receptor CCR5 (N-ter, ECL1, ECL2, ECL3) and C-terminal His⁶-tag, connecting with flexible turn-linker PGGS peptides. A preferred embodiment of such construct is illustrated in FIG. 3A [SEQ ID NOs: 31 & 32]. Like the method described above, this technique is based on “two-sided splicing by overlap extension” (Horton, R. M. et al., 1989) with some modifications. The adapted procedure is shown in FIG. 2. The PCR fragments coding for the N-term, linker, ECL1, linker (fragment 1a) and linker, ECL2, linker, ECL3, linker (fragment 1b) are generated in two separate primary PCRs. The first round of PCR was done with two pairs of overlapping long primers (1-F and 2-R for fragment 1a; 3-F and 4-R for fragment 1b) without the requirement of a CCR5 DNA template. The inner long primers (2 and 3) for the primary PCRs contain a 30 bp complementary region that allows the fusion of the two PCR fragments in the second PCR.

Fragments 1a and 1b are purified from the first PCR and used as templates with primers 5-F and 6-R. The two primary PCR products have compatible ends and these fragments are joined using overlap extension PCR. The subsequent cycles of the second PCR introduce the regulatory elements (Kozak sequence and C-terminal His-tag) to the joined DNA fragment using the outer primers 5-F and 6-R.

The scheme of extracellular domain-based and flexible turn linker PCR strategy of GPCRs adopted for CCR5 chemokine receptor are shown in FIGS. 3A and 3B.

### Example 4

### Expression and Purification of exCCR5 in E. coli

Our criteria for the design of soluble exCCR5 requires the formation of stable disulfide bonds for folding into a native conformation. A limitation of the production of correctly folded proteins in E. coli has been the relatively high reducing potential of the cytoplasmic compartment; disulfide bonds are usually formed only upon export into the periplasmic space. Bacterial strains with glutathione reductase (gor) and/or thioredoxin reductase (trxB) mutations enhance the formation of disulfide bonds in the E. coli cytoplasm (Prinz, W. A. et al., 1997; Aslund, F. et al., 1997). Also The Rosetta™ strains are designed to enhance the expression of eukaryotic proteins that contain codons rarely used in E. coli (Brinkmann, U., R. E. Mattes & P. Buckel, 1989; Seidel, H. M., D. L. Pompliano & J. R. Knowles, 1992; Kurland, C. & J. Gallant, 1996). These strains provide enhanced expression of target genes otherwise limited by the codon U.S.A.ge of E. coli.

For expression of exCCR5 chimera Rosetta-gami™ cells (Novagen) was used. To check the influence of N- and C-terminal tags on the stability and folding of exCCR5 and to provide the advantage of a double tag strategy for purification of protein from E. coli, we cloned exCCR5 in two different bacterial vectors.

To express a double-tagged (N-terminal-GST, and C-terminal 6xHis) exCCR5 protein, the final PCR product was subcloned into the polylinker of the bacterial expression vector pGEX-3, in frame with GST. Expression and purification of GST-exCCR5-6xHis tagged protein on glutathione-Sepharose beads were carried out (FIG. 4).

To express the exCCR5-6xHis tagged protein, the PCR final product was subcloned into the bacterial expression vector pET42a. ExCCR5 was purified using nickel chromatography utilizing the incorporated C-terminal His⁶-tag and eluted with 300 mM imidazole. Purity of the eluted protein was assessed using 15% or gradient 4-20% gels SDS-PAGE after extensive dialysis against phosphate buffered saline (PBS)-10% glycerol, in order to remove the imidazole (FIG. 5).

In both cases, the protein constructs were expressed in soluble form and appeared to retain the native structure of the extracellular domains. The presence of GST and hexahistidine (6xHis)-tagged CCR5 was confirmed by performing Western blot experiments probing the membrane with an anti-GST and anti-his5 antibody (FIG. 7). Analysis with anti-CCR5 conformation-dependent 2D7 mAb indicated that even in case of double tagged GST-exCCR5-6xHis protein, the extracellular domains form a stable and property folded unit.

Reducing SDS-PAGE gels indicated that factor Xa treatment proteolytically removed the N-terminal GST-tag and that the protein was present in two forms as a monomer (15 kDa, predictive M.W 13.156 KDa) and as a homodimer (30 kDa) (FIG. 4). The same monomer and homodimer forms were observed for the 6xHis C-terminal tagged protein exCCR5 6xHis (pET42a) (FIG. 5). The samples with a high concentration of protein on SDS-PAGE show not only monomer and homodimers but also demonstrated the tendency for formation of higher order tetramers (60 kDa).

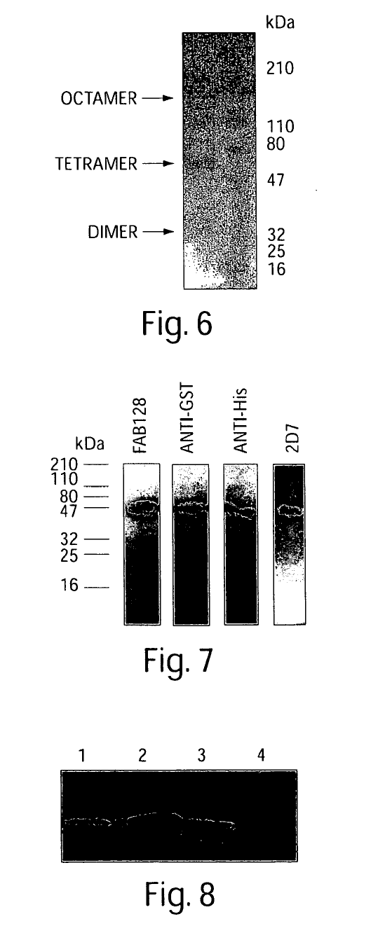

These results show heterologous expression of exCCR5-6xHis in prokaryotic systems. We next expressed the exCCR5 constructs in a mammalian system. A human exCCR5 receptor was subcloned via a polylinker into the eukaryotic expression vector phCMV-3 and stably transfected into CHO cells. Individual clones secreting CCR5 were selected by limiting dilution cloning. The product was immunoprecipitated from the supernatant with the 2D7 antibody (a conformation-dependent antibody against CCR5). SDS-PAGE resolved multiple forms of post translationally modified protein (glycosylated and possible sulfated), namely, dimer 32 kDa, tetramer 64 kDa and octamer 120 kDa) (FIG. 6).

### Example 5

### The Conformation-Dependent CCR5 mab, 2D7 Binds exCCR5

The conformation-dependent mAb, 2D7 was previously shown to recognize residues in ECL2 (Lee, B. et al., 1999), while mAbs PA9 and PA14 were shown to bind to amino acids in both the N-terminus and ECL2 (Olson, W. C. et al., 1999). The epitope recognized by mAb 2D7 on CCR5 has been partially mapped to the first half of the second extracellular loop (ECL-2) by mutagenesis studies (Olson, W. C. et al., 1999; Wu, L. et al., 1997). Amino acids K171, E172 were found to be critical for mAb 2D7 binding. But the epitope was determined to be conformation dependent, and the binding is lost in CCR5 mutants lacking the disulfide bridge between ECL-1 and ECL-2, as well as in reduced forms of CCR5 extracted from cells with various detergents (Mirzabekov, T. et al., 1999; Olson, W. C. et al., 1999).

Soluble GST-exCCR5 samples were immunoprecipitated with 2D7. After precipitation, the eluted protein was immunoblotted and developed using different mAbs. The presence of GST (N-terminal tag) and hexahistidine (6xHis)-C terminal tag in exCCR5 was confirmed by performing Western blot experiments probing the membrane with anti-GST and anti-His5 antibody (FIG. 7).

The presence of exCCR5 was confirmed by probing the membrane with anti-CCR5 mAbs, FAB182 and 2D7. The FAB182 mAbs recognized linear sequences within the C-terminal part of the ECL2 loop; 184 YSQYQF189. The 2D7 antibodies have been shown previously to bind non-reduced CCR5 on a Western blot but not to CCR5 where the cysteine residues have been reduced. The fact that these antibodies bind expressed non-reduced CCR5 in both immunoprecipitation assays and Western blots suggests strongly that the disulfide bridge between ECL-1 and ECL2 in our product is present and that exCCR5 is folded correctly.

### Example 6

### Binding of Chemokines to exCCR5

CCR5 binds several ligands MT-1alpha (CCL3), MIP-1beta (CCL4) and RANTES (CCL5). Physiological ligands, especially RANTES, have been shown to be effective inhibitors of CCR5 coreceptor activity (Simmons, G. et al., 1997).

Previous binding studies using monoclonal antibodies suggest that CCR5 interacts with chemokines through an exposed extracellular structural motif comprising part of the N-terminal domain and ECL2 (Olson, W. C. et al., 1999). In particular, it has been shown for human CCR5 that the negatively charged hydrophobic residues Asp11, Glu18, and Asp95 are critical in chemokine binding (Blanpain, C. et al., 1999). The alanine scanning of the amino-terminal domain shows that binding of RANTES is completely blocked if Asp11 and Glu18 are mutated (Blanpain, C. et al., 1999). Other charged residues such as Asp95 (ECL1), Arg168, Lys171, and Lys191 (ECL2) are also implicated in chemokine binding. Indeed, Arg168 is important for binding all agonists except RANTES. Further mutagenesis results show that chemokines also interact with CCR5 through the basic and hydrophobic residues Phe12, Arg17, Arg44, and Lys45 (and also Arg47 in the case of RANTES).

The presence of a disulfide bridge formed between two conserved cysteine residues on the first and second extracellular loop is a structural hallmark of the GPCR superfamily. Chemokine receptors, including CCR5, share two additional cysteine residues which are thought to form a disulfide bond between the N-terminus and the third extracellular loop. These two disulfide bridges probably impose a structural constraint on extracellular receptor domains and thereby stabilize a receptor conformation which is capable of ligand binding. Alanine mutation of any single extracellular cysteine in CCR5 resulted in reduced cell surface expression, loss of chemokine binding, and impaired HIV coreceptor function (Blanpain, C. et al., 1999). Naturally occurring CCR5 variants (C20S, C178R), with replacements of these critical cysteine residues, were identified in HIV-infected individuals, including a long-term non-progressor (Carrington, M. et al., 1997). In vitro data confirmed that these receptor mutants are defective in both chemokine binding and HIV-1 entry (Howard, O. M. et al., 1999).

In order to define the potency of RANTES binding to exCCR5 receptors, protein-protein interaction studies were carried out. ExCCR5 protein was immobilized on glutathione-sepharose beads by incubating the purified GST-protein fusions with glutathione-sepharose beads (Pharmacia), equilibrated in TEN100 (20 mM Tris, pH 7.4, 0.1 mM EDTA and 100 mM NaCl), or on Ni-NTA magnetic agarose beads and RANTES binding tested (FIG. 8). In a similar fashion, purified GST was bound to beads for a negative control. RANTES (3 pg) was incubated with the immobilized GST or GST-proteins and washed four times with TEN100. These protein-protein interaction assays provide molecular evidence that the physiological ligand RANTES binds to exCCR5 in both systems. Thus, the binding of RANTES to exCCR5 chimera, confirms the previous immunoprecipitation assays using 2D7 (a conformation-dependent antibody against CCR5) that demonstrates stable and properly folded extracellular domains of exCCR5.

### Example 7

### Involvement of exCCR5 Extracellular Domains in HIV-1 Coreceptor Activity: Specific Association of gp120/sCD4 Complexes with exCCR5

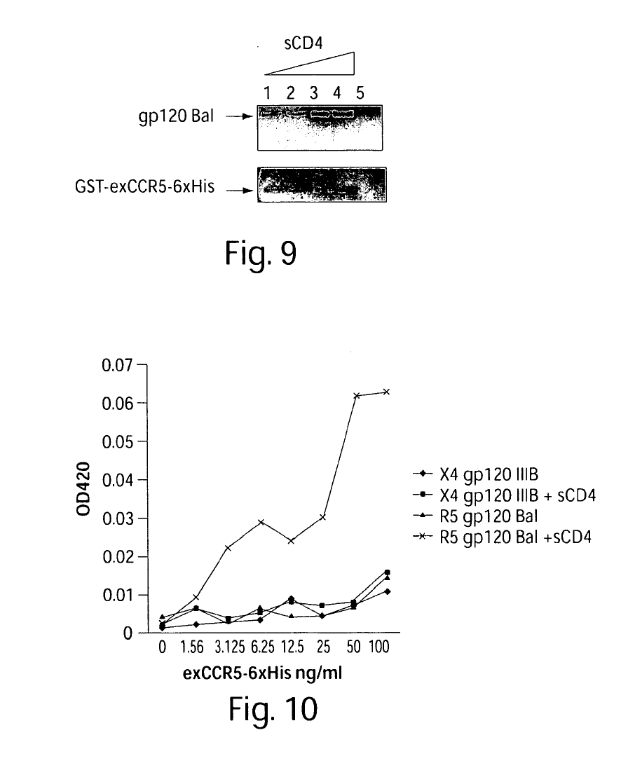

The site on HIV-1 envelope gp120 that binds coreceptors CCR5 or CXCR4 is not formed until CD4 is bound first. Recombinant gp120 envelope proteins from the CCR5-using HIV-1 isolate (BaL) were tested for binding to GST-exCCR5 chimera in presence or absence of sCD4 in a pull down assay. The gp120 efficiently bound to the GST-exCCR5-agarose only in the presence of sCD4 (FIG. 9). Binding conditions were optimized by varying the concentration of added sCD4. Binding was nearly undetectable when sCD4 was not present in the assay. Maximal binding of gp120 was obtained with a high concentration of sCD4.

Another approach to evaluate CD4-dependent binding of R5 gp120 to synCCR5 receptors was ELISA (FIG. 10). A binding assay was performed by adding an increasing amount of R5 (Bal) or X4 (IIIB) gp120 proteins to synCCR5 immobilized on Ni plates in the presence or absence of sCD4. Total binding was determined in presence of 500 ng sCD4. R5-tropic envelope protein (Bal) bound to synCCR5 in the presence of sCD4 and there was no effective binding in the absence of sCD4. CXCR4-using envelope proteins (IIIB) did not bind to exCCR5 the presence or absence of sCD4.

These studies, showing CD4-dependent binding of R5 gp120 to exCCR5 are in agreement with the results presented by others that infection via CCR5 and gp120 binding to CCR5 is strictly dependent on the presence of the CD4 receptor or sCD4 (Doranz, B. J., S. S. Baik & R. W. Doms, 1999; Martin, K. A. et al., 1997; Trkola, A. et al., 1999; Wu, L. et al., 1997; Moore, J. P. & J. Sodroski, 1996). These findings confirmed that extracellullar CCR5 receptor domains in exCCR5 chimera retain the native, stable conformation of full length 7™ receptor which allows CD4-dependent binding of gp120.

### Example 8

### Chimeric, Multi-Targeting Analog of Soluble GPCR Peptide

A chimeric protein that includes three functional elements that will interact at each step during HIV-1 entry of cells is described. The new construct combines the advantages of three types of entry inhibitor currently available which target envelope-CD4 and envelope-coreceptor interactions, as well as a late step in fusion. This new chimeric protein combines the advantages of three types of current entry inhibitors (CD4 receptor inhibitors, chemokine receptor inhibitors and inhibitors of membrane fusion) in one protein thereby allowing simultaneous targeting of three different steps in virus entry that restricts the emergence of resistant viruses. The chimera will mimic the natural process of virus entry by interacting with multi targets of envelope gp120-gp41 proteins.

A soluble HIV entry inhibitor was constructed, which comprises a protein containing three functional elements: (i) two first domains of CD4 D1-D2 (primary receptor of HIV); (ii) a soluble extracellular analog of the CCR5 or CXCR4 chemokine receptors: and, (iii) a gp41 ectodomain (628-683) which includes the HR2 region (628-661) and the tryptophan-rich membrane proximal external region (MPER) (665-683). These elements are connected by flexible linkers to allow the adoption of the most natural, ligand-binding conformations.

The first structural element of the chimera is based on CD4 (D1-D2) immunoglobulin-like domains. CD4 contains four immunoglobulin-like domains termed D1, D2, D3 and D4. The env-binding site has been localized to D1, while other regions are also considered important in governing the flexibility, conformation and function of CD4. The first structural element of our soluble chimera (i.e. D1-D2 of CD4) is designed to attach to gp120 on virus particles and compete with cell surface CD4 for binding virus particles.

The second structural element of our chimera is derived from (ExCCR5/exCXCR4) soluble extracellular coreceptor analogs. CD4 and CCR5 are physically associated on the cell surface even in the absence of gp120 glycoprotein (Xiao, X. et al., 1999; Lapham, C. K. et al., 1999). This association has been demonstrated to be mediated through interactions of the second extracellular loop of CCR5 with the first two domains of CD4. As expected, soluble CD4 (D1-D4, no transmembrane domain) also interacts with the chemokine receptor CCR5 (Wang, X. & R. Staudinger, 2003). For purposes of designing a multi-domain chimera for inhibiting HIV entry, soluble exCCR5 and exCXCR4 proteins are used. Each of ExCCR5 and exCXCR4 contains the N-terminus, ECL1, ECL2, and ECL3 extracellular domains of human chemokine receptors. The modified protein maintains the overall three-dimensional structure of the extracellular portion of wild type chemokine receptors. These constructs do not contain the transmembrane regions (TM1-TM7), the intracellular domains (i1, i2, i3) or the cytoplasmic regions of CCR5 and CXCR4. For this particular example, the constructs contain flexible short turn PGGS linkers inserted at sites between the extracellular domains of chemokine receptors (N-term, ECL1, ECL2, ECL3) and a His⁶-tag at carboxyl end. ExCCR5 and exCXCR4 were expressed in E. coli as a soluble monomer and homodimer forms. ExCCR5 was immunoprecipitated with 2D7 (an ECL2-specific, conformation-dependent antibody against CCR5). ExCCR5 also binds specifically to RANTES, a physiological ligand of CCR5 and specifically associates with gp120/sCD4 complexes. CCR5-using envelope proteins bound to exCCR5 in the presence of sCD4 with little binding in the absence of sCD4. CXCR4-using envelope proteins did not bind to exCCR5 in the presence or absence of sCD4. The functional assays of exCCR5 using conformation dependent antibodies, physiological ligands and R5 HIV-1 envelopes demonstrate that exCCR5 forms are stable and correctly folded. The pairing of D1-D2 (CD4) to the N-terminus of a soluble chemokine receptor analogs is designed to result in a protein that binds to envelope spikes on the surface of virions and induce the conformational changes that result in the exposure of the fusion domain. If these changes occur away from a cell surface then we expect the fusion domain to embed in the nearest hydrophobic environment and effectively neutralize the virus.

The third structural element of chimera is the fusion inhibitor ectodomain of gp41. The interaction between the 120-CD4 complex and coreceptors (CCR5 or CXCR4), induce conformational changes that cause a shift from a non-fusogenic to a fusogenic state of the HIV gp41 and ultimately drives the fusion process. The fusion peptide (FP) at the N-terminus of gp41 is exposed and inserted into the cell membrane. Then, gp41 undergoes a structural reorganization that provokes the interaction between the heptad repeat regions, HR1 and HR2, to form a thermostable, six-helix bundle structure, which is critical for viral and cellular membrane fusion. The change in free energy associated with the formation of the six-helix bundle provides the force necessary for the formation of the fusion pore, which widens and allows the viral capsid to enter the target cell. Enfuvirtide is a synthetic peptide of 36 amino acids that mimics an HR2 fragment of gp41. It binds to the HR1 region and blocks the formation of the six-helix bundle structure, which is critical for the fusion process. We have therefore added the gp41 ectodomain (628-683) to the C-terminal end of coreceptor analog through a PGGS flexible linker [SEQ ID NO:1]. The gp41 ectodomain of CD4-CCR5-gp41 chimera consists of the important functional region (628-683) which includes C34 (628-661) and the tryptophan-rich membrane proximal external region (MPER, 665-683). These regions contain epitopes for the broadly active neutralizing monoclonal antibodies, 2F5 and 4E10. We hypothesized that the ectodomain (628-683) will mimic the HR2 domain in gp41 and will block the formation of the six-helix bundle structure, further disrupting the HIV entry process.

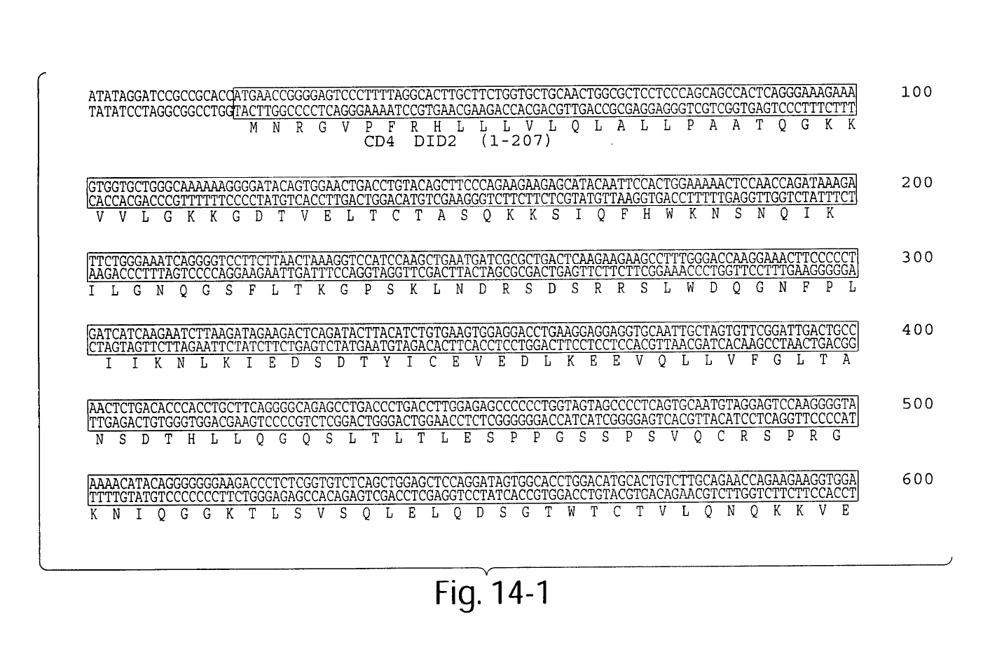

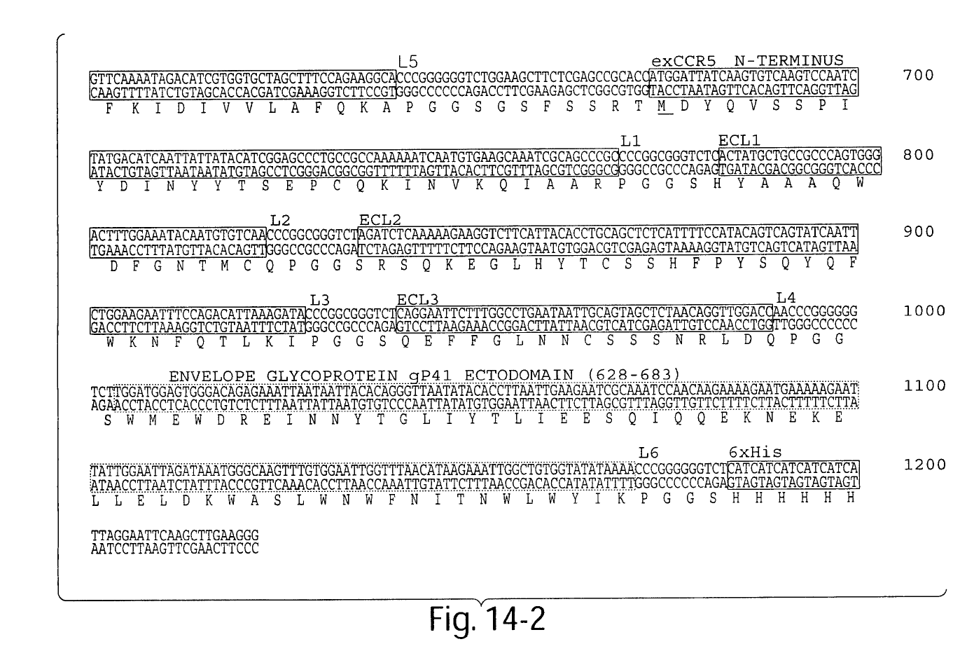

The cloning strategy used to construct the chimeric analog of soluble GPCR peptide is as follows: The first structural element, CD4-D1D2, consisting of a signal peptide and domains 1 and 2 of CD4 (residues 1-207) was cloned upstream of the N-terminus of exCCR5 (M218-Q324). The D1D2 domains of CD4 were joined to exCCR5 by a flexible turn-like linker PGGSGSFSSRT (L5) [SEQ ID NO:34]. The second element consisting of the chemokine receptor analog, exCCR5, encodes for the N-terminus and 3 extracellullar loops (ECL1, ECL2, ECL3) of CCR5, each of which are joined via four amino acid (PGGS) linkers (L1, L2, L3 L4). The third element is the gp41 ectodomain (residues 628-683) which is joined by a flexible turn-like linker PGGS (L6). The gp41 ectodomain includes residues of HR2 as well as the tryptophan-rich membrane proximal external region of gp41 (MPER; residues 665-683). These regions contain the epitopes for the broadly neutralizing human monoclonal antibodies, 2F5 and 4E10. A preferred embodiment of the synthetic gene encoding for the CD4-_{D1D2}-exCCR5-gp41(628-683)-6xHIS soluble chimeric protein is dipicted in FIG. 14 [SEQ ID NO:37].

HIV-1 drugs that targeted single events in the replication cycle e.g. reverse transcriptase inhibitors, effectively reduced viral loads in vivo and delayed disease progression. However, these therapies were rapidly overcome by resistant viral variants that emerged and predominated in a short period of time (weeks to months depending on the drug). Current combination therapies target two or three viral proteins and make it much more unlikely for viral variants to evolve that are simultaneously resistant to the different drugs. In this application, we have described a single protein construct that will simultaneously target several events during viral entry. We hypothesize that the chimeric constructs described will mimic natural processes during virus entry by interacting with multiple targets of envelope gp120-gp41 proteins. Three critical events that lead to the fusion cascade will be inhibited by the chimeric protein; (1) CD4 N-terminal D1-D2 domains of chimera will mimic the CD4 receptor and will inhibit attachment of virus to target cells, (2) A soluble extracellular coreceptor analog of chimera will mimic chemokine coreceptor and inhibit attachment of the virus to a cell surface coreceptor, and (3) a gp41 C-terminal helical peptide in the chimera will inhibit gp41 conformational changes that lead to the formation of the six-helix bundle structure required for fusion. We predict that the combination of these three structural elements with different mechanisms of action into one protein will greatly increase inhibitory potency as well as limiting the likelihood of virus resistance.

### Example 9

### Use of exCCR5 and exCXCR4 for an HIV Vaccine and Production of Neutralizing Monoclonal Antibodies to Highly Conserved Coreceptor-Binding Site of gp120 and the Membrane Proximal Region Present on gp41

The generation of an antibody response capable of neutralizing a broad range of clinical isolates remains an important goal of human immunodeficiency virus type 1 (HIV-1) vaccine development. Envelope glycoprotein (Env)-based vaccine candidates will also need to take into account the extensive genetic diversity of circulating HIV-1 strains. We describe here the generation of soluble forms of chemokine receptors that will be helpful for production and characterization of high affinity monoclonal or humanized antibodies to highly conserved epitopes on HIV-1 envelope glycoproteins.

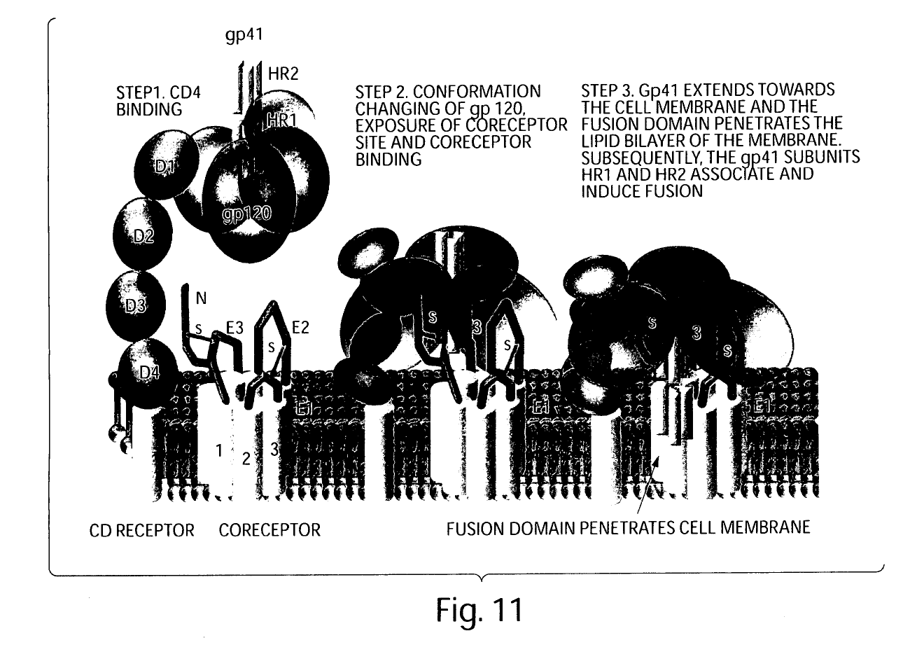

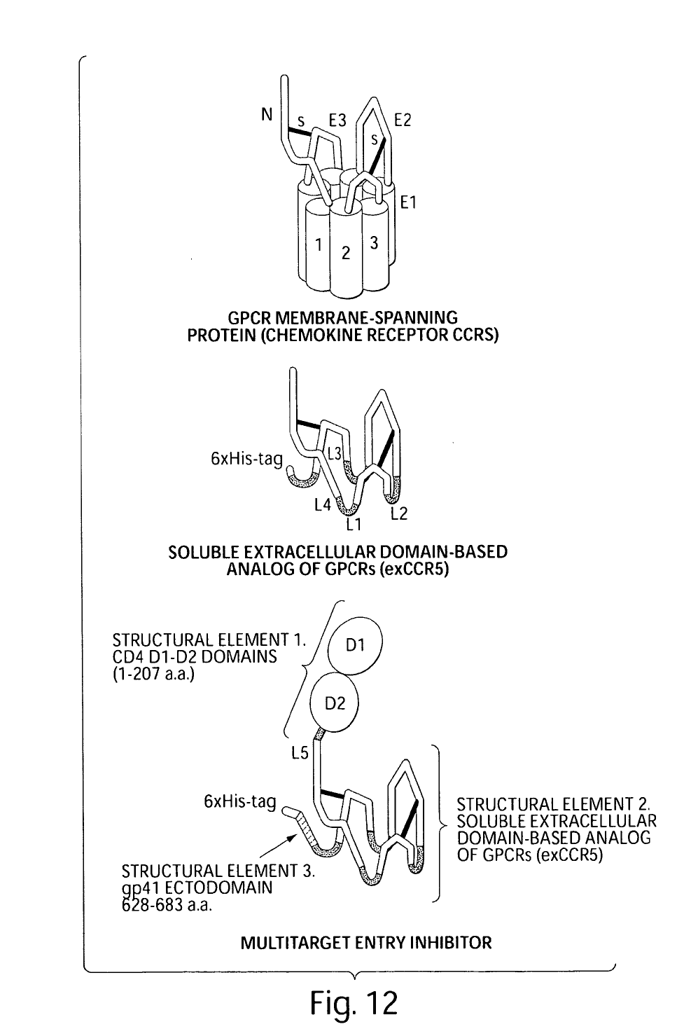

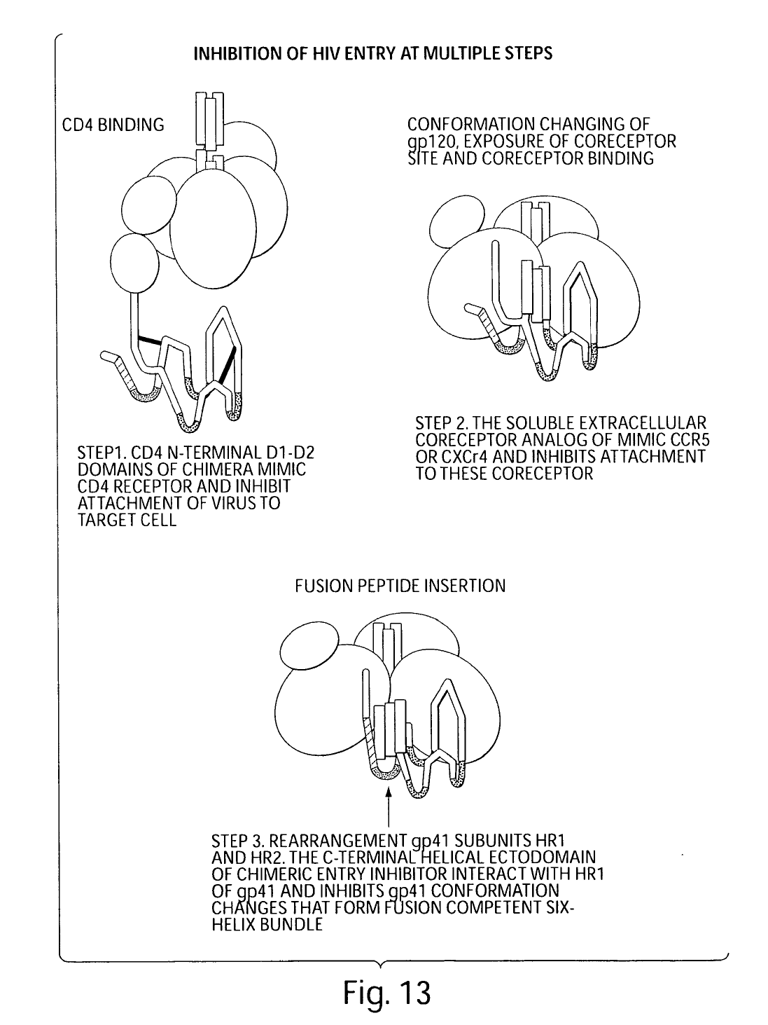

The connection between HIV and the chemokine system has implications for the development of an effective HIV vaccine. Current vaccine studies generally assume that a vaccine will induce both cellular immunity and neutralizing antibodies. The latter task is hampered by the fact that the only target for antibody induction is the envelope gene, which also displays the highest sequence diversity. After attachment of gp120 to CD4, the coreceptor-binding site on gp120 is formed and exposed. The coreceptor binding site consists of the conserved beta strands of the bridging sheet and determinants on the V3 loop. V3 loop amino acids on gp120 determine whether CCR5 and/or CXCR4 are used. The coreceptor binding site of gp120 and the membrane proximal region on gp41 contain highly conserved neutralizing antibody epitopes. However, antibody accessibility to such regions is hindered by diverse protective mechanisms, including shielding by variable loops, conformational flexibility and extensive glycosylation. HIV has evolved a unique strategy of interaction with its cellular receptors CCR5 and CXCR4, which provides an effective mechanism for concealing highly conserved neutralization epitopes from the attack of host antibodies. The two-stage receptor-interaction strategy allows HIV-1 to maintain the highly conserved coreceptor-binding surface in a cryptic conformation, unraveling it only upon binding of gp120 to CD4. However, this occurs in a sterically and temporally constrained setting in close proximity to the cellular membrane, beyond the reach of complete antibody molecules (Labrijn et al, 2003). The detection, albeit infrequent, of primary strains of HIV-1 (Zerhouni et al, 2004; Decker et al, 2005) and HIV-2 (Reeves et al, 1999) capable of infecting coreceptor-expressing cells in a CD4-independent fashion has led the idea of an ancestral HIV that could directly bind to coreceptors without requiring CD4 (FIG. 11).

The Achilles' heel of this putative ancestor and its present-day descendants is their marked sensitivity to antibody-mediated neutralization due to a constitutive exposure of the coreceptor-binding region (Kolchinsky et al, 2001). Consistent with this concept, infected individuals possess high titers of antibodies specific for this region (Decker et al, 2005), most likely elicited by shed monomeric gp120 complexed with cell-surface CD4. Such antibodies will continuously patrol against the in vivo emergence of CD4-independent variants. Thus, despite its critical role in the viral entry process and its documented immunogenicity in humans (Decker et al, 2005), this region is generally discounted as a vaccine target. Nevertheless, some epitopes overlapping or neighboring the coreceptor-binding surface are at least partially accessible in the native, CD4-unbound envelope oligomer (Moulard et al, 2002; Labrijn et al, 2003), providing a basis for the use of the CD4-triggered envelope or rationally designed synthetic immunogens mimicking this region as a means to induce broadly protective antibodies. Therefore, exCCR5 and exCXCR4 plus soluble CD4 or CD4-exCCR5 chimera may serve as conformational framework to stabilize a specific neutralizing epitope in order to induce such an immunological response upon immunization.

### Example 10

### Screening Therapeutic Candidates Based on Soluble GPCR Constructs

Here, we describe different approaches for proteomic screening. The findings described herein showed that of exCCR5-6xHis in E. coli was expressed in soluble form. Using conformation dependent antibodies, physiological ligands and R5 HIV-1 envelopes we demonstrated that exCCR5 is stable, likely to be correctly folded and can perform many of the same interactions as the native CCR5 receptor. Protein interactions that are important for disease processes are likely to form specific targets for therapeutics. The two-hybrid system has been very useful in the identification of such targets in high-throughput proteomic screens. The same two-hybrid strategy is particularly useful for the identification of novel (candidate) proteins from cDNA libraries, which interact with a extracellular domain GPCRs proteins, and for the subsequent determination of protein domains or amino acids critical for the interaction.

The finding that some chemokines and their receptors are upregulated in both acute and chronic inflammatory diseases, and that they are key players in the development of AIDS, has provided the pharmaceutical industry with new targets for therapeutic intervention in these diseases. Several approaches are being developed to block the effects of chemokines, including small-molecule antagonists of chemokine receptors, modified chemokines and antibodies directed against chemokine receptors. Chemokines and their receptors are excellent targets and GPCRs are targeted by 50% of medicines that are marketed currently. Nonetheless, GPCRs are generally difficult to identify antagonists for and to evaluate binding sites. Unfortunately, GPCRs, like other membrane-embedded proteins, have characteristics that make their 3D structure extremely difficult to determine experimentally. Therefore, the main ways to investigate the properties of GPCRs and their interaction with ligands are currently based on site-directed mutagenesis or molecular modeling techniques. Another important point is the cognate ligands for the majority of GPCRs have not yet been found.

### Example: 11

### High-Throughput-Screening of Antagonists for GPCRs

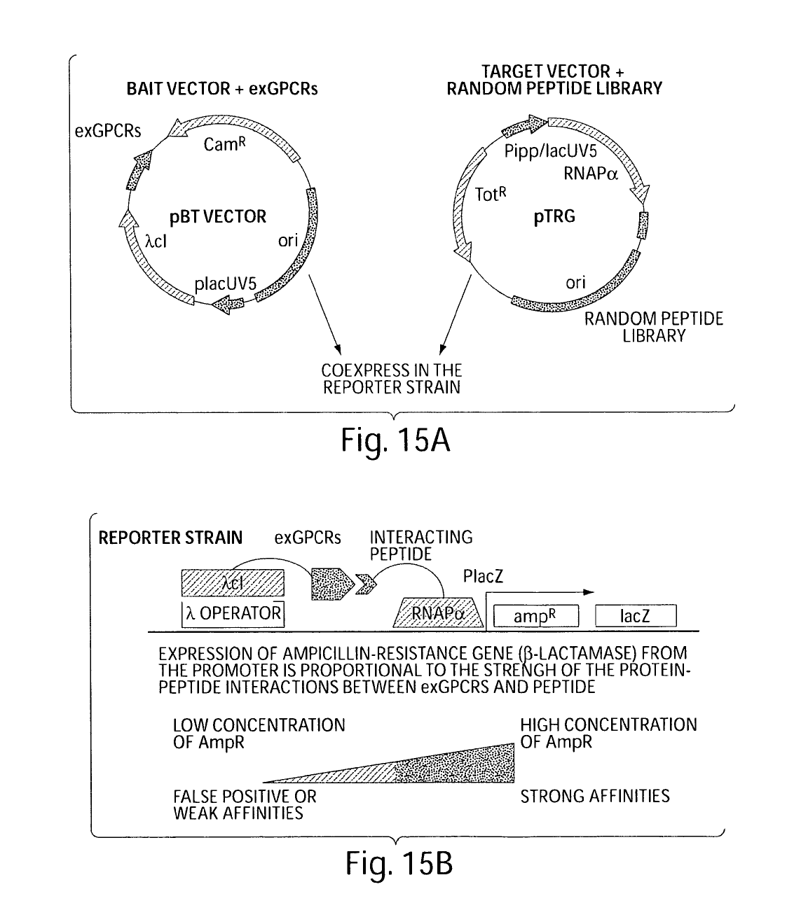

As illustrated in FIG. 15, for fast detection of peptides with strong affinity and specificity to extracellular domains of GPCRs, exCCR5 (or any soluble GPCR polypeptide of the instant invention) is cloned into the pBT bait vector next to the carboxyl-terminal end of the phage cI protein to form a fusion protein. The pTRG prey plasmid encodes the random peptide library fused to the amino-terminal domain of the subunit of RNA polymerase. The bacterial reporter strain harbors an F′ episome containing a lacUV5 promoter-operator region directing the expression of the amp^{R} gene, conferring resistance to carbenicillin. A operator sequence located upstream of this promoter provides a binding site for the bait protein. The interaction of bait and prey proteins recruits RNA polymerase to the promoter of the reporter gene, activating transcription. In the absence of activation, basal transcription results in a low level of carbenicillin resistance; elevated resistance requires activation of the promoter by bait and prey proteins. Expression from the test promoter of the reporter cassette is proportional to the strength of the protein-protein interaction between bait exGPCRs and target (random peptide library). The carbenicillin concentration can be adjusted to screen for strong affinity high specific protein-protein interactions. The lacZ gene serves as secondary reporter providing a visible phenotype for identifying positive protein-protein interactions.

Efficient high-throughput expression of genes in E. coli or yeast cells can facilitate large-scale functional genomic studies searching for GPCR antagonists or agonists. The two-hybrid method uses the restoration of transcriptional activation to indicate the interaction between two proteins. Because it is performed in a microbial system, the two-hybrid assay can be used to rapidly select-out GPCR specific peptides from several million candidate clones in the random peptide library. Using this method, peptides can be identified that discriminate between wild-type and mutant forms of a target exGPCRs domain protein or that inhibit specific protein-protein interactions. These applications may facilitate the search for drugs or other ligands that have an affinity for selected protein. Using the bacterial two-hybrid system we expect to find positive clones encoding peptides that have the ability to bind to extracellular domains of exCCR5 and exCXCR4 in vivo. The main problem encountered with the two-hybrid system is the high frequency of false positive interactions. Novel peptides detected will therefore require further validation. This includes obtaining direct evidence of physical interaction, using techniques such as co-immunoprecipitation and immunofluorescence to demonstrate that the two proteins can form a complex under physiological conditions. The mammalian two-hybrid system will be used to confirm protein-protein interactions with the ‘correct’ environment for a mammalian protein.

One with ordinary skill in the art will realize that the instant invention is suitable for high-throughput, large-scale, functional genomic studies with GPCRs. The same two-hybrid strategy described above is particularly useful for the identification of novel (candidate) proteins from cDNA libraries, which interact with a extracellular domain GPCRs proteins, and for the subsequent determination of protein domains or amino acids critical for the interaction. cDNA libraries can be used that are specific for species-, tissue-, or development stage mRNA expression. Specific mutations, insertions, or deletions that affect the encoded amino acids can be introduced into DNA encoding the target protein, and the mutant target proteins can be assayed for the protein-protein interaction with the bait protein. Interpretation of these data may be useful for elucidating effects on ligand binding, receptor activation, desensitization and trafficking, as well as receptor signaling.

This pervasive involvement in normal biological processes has the consequence of involving GPCRs in many pathological conditions (such as hypertension, cardiac dysfunction, depression, anxiety, obesity, inflammation, and pain). Our invention will therefore lead to novel approaches to screen for therapeutic and diagnostic tools.

### REFERENCES

Agace, W. W. et al. (2000) Constitutive expression of stromal derived factor-1 by mucosal epithelia and its role in HIV transmission and propagation. Curr Biol. 10: 325-328.
Alcami, A. et al. (1992) A soluble receptor for interleukin-1 encoded by vaccinia virus: a novel mechanism of virus modulation of the host response to infection. Cell 71:153.
Aleshin, A. E. et al. (1998) Regulation of hexokinase I: crystal structure of recombinant human brain hexokinase complexed with glucose and phosphate. J. Mol. Biol. 282: 345-357.
Alfthan, K. et al. (1995) Properties of a single-chain antibody containing different linker peptides. Protein Eng. 8: 725-731.
Allaway, G. P. et al. (1995) Expression and characterization of CD4-IgG2, a novel heterotetramer that neutralizes primary HIV type 1 isolates. AIDS Res Hum Retroviruses 5. 11(5): 533-9.
Argos, P. (1990) An investigation of oligopeptides linking domains in protein tertiary structures and possible candidates for general gene fusion. J. Mol. Biol. 211: 943-958.
Ashkenazi, A. et al. (1990) Mapping the CD4 binding site for human immunodeficiency virus by alanine-scanning mutagenesis. Proc Natl Acad Sci U.S.A. 87(18): 7150-4.
Aslund, F. et al. (1997) Redox potentials of glutaredoxins and other thiol-disulfide oxidoreductases of the thioredoxin superfamily determined by direct protein-protein redox equilibria. J Biol Chem. 272(49): 30780-6.
Baba, M. et al. (1999) A small-molecule, nonpeptide CCR5 antagonist with highly potent and selective anti-HIV-1 activity. Proc Natl Acad Sci U.S.A. 96(10): 5698-703.
Baba, M. et al. (2005) TAK-652 inhibits CCR5-mediated human immunodeficiency virus type 1 infection in vitro and has favorable pharmacokinetics in humans. Antimicrob Agents Chemother. 49(11): 4584-91.
Baggiolini, M., (1998) Chemokines and leukocyte traffic. Nature 392(6676): 565-8.
Baird, A. M. et al. (1999) The role of cytokine receptor signaling in lymphocyte development. Curr Opin Immunol. 11(2): 157-66.
Balaram, P. (1999) De novo design: backbone conformational constraints in nucleating helices and beta-hairpins. J. Peptide Res. 54: 195-199.
Baneyx, F. and Mujacic, M. (2004) Recombinant protein folding and misfolding in Escherichia coli. Nat Biotechnol 22: 1399-408.
Benach, J. et al. (1998) The refined crystal structure of Drosophila lebanonensis alcohol dehydrogenase at 1.9 A resolution. J. Mol. Biol. 282: 383-399.
Berger, E. A., et al., (1998) A new classification for HIV-1. Nature 391(6664): 240.
Berger, E. A. et al. (1999) Chemokine receptors as HIV-1 coreceptors: roles in viral entry, tropism, and disease. Annu Rev Immunol. 17: 657-700.
Blanpain, C. et al. (1999) Multiple charged and aromatic residues in CCR5 amino-terminal domain are involved in high affinity binding of both chemokines and HIV-1 Env protein. J Biol Chem. 274(49): 34719-27.
Blanpain, C. et al. (2002) CCR5 and HIV infection. Receptors Channels 8(1): 19-31.
Brelot, A. et al. (1997) Role of the first and third extracellular domains of CXCR-4 in human immunodeficiency virus coreceptor activity. J Virol 71(6): 4744-51.
Brinkmann, U. et al. (1989) High-level expression of recombinant genes in Escherichia coli is dependent on the availability of the dnaY gene product. Gene 85(1): 109-14.
Broxmeyer, H. E. and Kim, C. H. (1999) Regulation of hematopoiesis in a sea of chemokine family members with a plethora of redundant activities. Exp Hematol 27(7): 1113-23.
Cabrita, L. D. et al. (2004) Protein expression and refolding—A practical guide to getting the most out of inclusion bodies. Biotechnol Annu Rev 10: 31-50.
Canagarajah, B. J. et al. (1997) Activation mechanism of the MAP kinase ERK2 by dual phosphorylation. Cell 90: 859-869.
Carrington, M., et al. (1997) Novel alleles of the chemokine-receptor gene CCR5. Am J Hum Genet. 61(6): 1261-7.
Castonguay, L. A. et al. (2003) Binding of 2-aryl-4-(piperidin-1-yl)butanamines and 1,3,4-trisubstituted pyrrolidines to human CCR5: a molecular modeling-guided mutagenesis study of the binding pocket. Biochemistry 42(6): 1544-50.
Chalmers, J. J. et al. (1990) Effects of temperature on Escherichia coli overproducing beta-lactamase or human epidermal growth factor. Appl Environ Microbiol. 56: 104-11.
Chao, B. H. et al. (1989) A 113-amino acid fragment of CD4 produced in Escherichia coli blocks human immunodeficiency virus-induced cell fusion. J Biol Chem, 264(10): 5812-7.
Chou, T. C. and Talalay P. (1981) Generalized equations for the analysis of inhibitions of Michaelis-Menten and higher-order kinetic systems with two or more mutually exclusive and nonexclusive inhibitors. Eur J Biochem. 115(1): 207-16.
Clayton, L. K. et al. (1989) Identification of human CD4 residues affecting class II MHC versus HIV-1 gp120 binding. Nature 339(6225): 548-51.
Cleland, J. L. (1993) Impact of Protein Folding on Biotechnology.
Cocchi, F, et al. (1995) Identification of RANTES, MIP-1, and MT-1 as the major HIV-suppressive factors produced by CD8+ T cells. Science 270: 1811-1815.
Colamonici, O. R. et al. (1995) Vaccinia virus B18R gene encodes a type I interferon-binding protein that blocks interferon transmembrane signaling. J. Biol. Chem 270: 15974.
Combadiere, C. et al. (1996) Cloning and functional expression of CC CKR5, a human monocyte CC chemokine receptor selective for MIP-1(alpha), MIP-1(beta), and RANTES. J Leukoc Biol, 60(1): 147-52.
Couture, L., et al., (1996) A defined epitope on the human choriogonadotropin alpha-subunit interacts with the second extracellular loop of the transmembrane domain of the lutropin/choriogonadotropin receptor. Eur J Biochem 241(2): 627-32.
Crasto C. J. and Feng, J. (May 2000) LINKER: a program to generate linker sequences for fusion proteins. Protein Engineering 13(5): 309-312.
Cunnion, K. M. (1999) Tumor necrosis factor receptors encoded by poxviruses. Mol. Genet. Metab. 67:278.
Davis, G. D. et al. (1999) New fusion protein systems designed to give soluble expression in Escherichia coli. Biotechnol Bioeng. 65: 382-8.
De Clercq, E. (2005) New approaches toward anti-HIV chemotherapy. J Med Chem. 48(5): 1297-313.
de Vreese, K. et al. (1996) The molecular target of bicyclams, potent inhibitors of human immunodeficiency virus replication. J Virol., 70(2): 689-96.
Decker, J. M. et al. (2005) Antigenic conservation and immunogenicity of the HIV coreceptor binding site. J Exp Med. 201: 1407-1419.
Delgado, M. B. et al. (2001) Rapid inactivation of stromal cell-derived factor-1 by cathepsin G associated with lymphocytes. Eur J Immunol. 31: 699-707.
Deng, H. et al. (1996) Identification of a major co-receptor for primary isolates of HIV-1. Nature 381(6584): 661-6.
Derman, A. I. et al. (1993) Mutations that allow disulfide bond formation in the cytoplasm of Escherichia coli. Science 262: 1744-7.

Dewan, J. C. et al. (1994) Crystal structure of kappa-bungarotoxin at 2.3-A resolution. Biochemistry 33: 13147-13154.
Diederichs, K. et al. (1991) The refined structure of the complex between adenylate kinase from beef heart mitochondrial matrix and its substrate AMP at 1.85 A resolution. J. Mol. Biol. 217: 541-549.
Doi, M. et al. (1990) Conformational characteristics of opioid kappa-receptor agonist: crystal structure of (5S,7S,8S)-(−)-N-methyl-N-[7-(1-pyrrolidinyl)-1-oxaspiro[4.5]dec-8-yl]benzeneacetamide (U69,593), and conformational comparison with some kappa-agonists. Chem Pharm Bull (Tokyo) 38(7): 1815-8.
Donzella, G. A. et al. (1998) AMD3100, a small molecule inhibitor of HIV-1 entry via the CXCR4 co-receptor. Nat Med. 4(1): 72-7.
Doran, B. J. et al. (1999) Use of a gp120 binding assay to dissect the requirements and kinetics of human immunodeficiency virus fusion events. J Virol. 73(12): 10346-58.
Dorr, P. et al. (2005) Maraviroc (UK-427,857), a potent, orally bioavailable, and selective small-molecule inhibitor of chemokine receptor CCR5 with broad-spectrum anti-human immunodeficiency virus type 1 activity. Antimicrob Agents Chemother. 49(11): 4721-32.
Dove, S. L. et al. (1997) Activation of prokaryotic transcription through arbitrary protein-protein contacts. Nature 386: 627-630.

Dove, S. L. Hochsild, A. (Mar. 1, 1998) Conversion of the subunit of Escherichia coli RNA polymerase into a transcriptional activator or an activation target. Genes & Development. 12(5): 745-754.
Dragic, T. et al. (1998) Amino-terminal substitutions in the CCR5 coreceptor impair gp120 binding and human immunodeficiency virus type 1 entry. J Virol. 72(1): 279-85.
Dragic, T. et al. (2000) A binding pocket for a small molecule inhibitor of HIV-1 entry within the transmembrane helices of CCR5. Proc Natl Acad Sci U.S.A. 97(10): 5639-44.
Elgavish, S. et al. (1998) Structures of the Erythrina corallodendron lectin and of its complexes with mono- and disaccharides. J. Mol. Biol. 277: 917-932.
Ewalt, K. L. et al. (1997) In vivo observation of polypeptide flux through the bacterial chaperonin system. Cell 90: 491-500.
Fahnert, B. et al. (2004) Inclusion bodies: formation and utilisation. Adv Biochem Eng Biotechnol. 89: 93-142.
Fan, Z.-C. et al. Three-dimensional structure of an Fv from a human IgM immunoglobulin. J. Mol. Biol. 228: 188-207.
Farzan, M., et al. (1998) A tyrosine-rich region in the N terminus of CCR5 is important for human immunodeficiency virus type 1 entry and mediates an association'between gp120 and CCR5. J Virol. 72(2): 1160-4.
Fedorov, A. N. and Baldwin, T. O. (1997) Cotranslational protein folding. J Biol Chem. 272: 32715-8.
Feng, Y. et al. (1996) HIV-1 entry cofactor: functional cDNA cloning of a seven-transmembrane, G protein-coupled receptor. Science 272(5263): 872-7.
Fitzpatrick, V. D. and Vandlen, R. L. (1994) 6agonist selectivity determinants in somatostatin receptor subtypes I and II. J Biol Chem. 269(40): 24621-6.
Galloway, C. A. et al. (2003) Increasing the yield of soluble recombinant protein expressed in E. coli by induction during late log phase. Biotechniques 34: 524-6, 528, 530.
George, R. A. and Hering a J. (2003) An analysis of protein domain linkers: their classification and role in protein folding. Prot. Engineer. 15: 871-879.
Georgiou, G. and Valax, P. (1986) Localization of inclusion bodies in Escherichia coli overproducing beta-lactamase or alkaline phosphatase. Appl Environ Microbiol. 52: 1157-61.
Georgiou, G. et al. (1996) Expression of correctly folded proteins in Escherichia coli. Curr Opin Biotechnol. 7: 190-7.
Grzesiek, S. et al. (1997) Refined solution structure and backbone dynamics of HIV-1 Nef. Protein Sci. 6: 1248-1263.
Guo, Q., et al., (2003) Biochemical and genetic characterizations of a novel human immunodeficiency virus type 1 inhibitor that blocks gp120-CD4 interactions. J Virol. 77(19): 10528-36.

Havukainen, R. et al. (1996) Covalent binding of three epoxyalkyl xylosides to the active site of endo-1,4-xylanase II from Trichoderma reesei. Biochemistry 35: 9617-9624.
Hedrick, J. A. and Zlotnik, A. (1996) Chemokines and lymphocyte biology. Curr Opin Immunol. 8(3): 343-7.
Helfrich, W. (1998) Construction and characterization of a bispecific diabody for retargeting T cells to human carcinomas. Int. J. Cancer. 76: 232-239.
Hill, C. M. et al. (1998) The amino terminus of human CCR5 is required for its function as a receptor for diverse human and simian immunodeficiency virus envelope glycoproteins. Virology 248(2): 357-71.
Hockney, R. C. (1994) Recent developments in heterologous protein production in Escherichia coli. Trends Biotechnol. 12: 456-63.
Horn, F. and Vriend, G. (1998) G protein-coupled receptors in silico. J Mol Med. 76(7): 464-8.
Horton, R. M. et al. (1989) Engineering hybrid genes without the use of restriction enzymes: gene splicing by overlap extension. Gene 77(1): 61-8.
Howard, O. M. et al. (1999) Naturally occurring CCR5 extracellular and transmembrane domain variants affect HIV-1 Co-receptor and ligand binding function. J Biol Chem. 274(23): 16228-34.
Hoxie, J. A. (1991) Hypothetical assignment of intrachain disulfide bonds for HIV-2 and SW envelope glycoproteins. AIDS Res Hum Retroviruses 7(6): 495-9.
Hu, F. Q. et al. (1994) Cowpox virus contains two copies of an early gene encoding a soluble secreted form of the type II TNF receptor. Virology 204: 343.
Hull, K. M. et al. (2002) The TNF receptor-associated periodic syndrome (TRAPS): emerging concepts of an autoinflammatory disorder. Medicine 81: 349.
Imamura, H. et al. (1999) High level expression of Thermococcus litoralis 4-alpha-glucanotransferase in a soluble form in Escherichia coli with a novel expression system involving minor arginine tRNAs and GroELS. FEBS Lett. 457: 393-6.
Jackson, S. E. (1998) How do small single-domain proteins fold? Fold Des 3: R81-91.
Jameson, B. A. et al. (1988) Location and chemical synthesis of a binding site for HIV-1 on the CD4 protein. Science 240(4857): 1335-9.
Junghans, R. P. et al. (1996) Metabolism of Tac (IL2R): physiology of cell surface shedding and renal catabolism, and suppression of catabolism by antibody binding. J. Exp. Med. 183: 1587.
Kabsch, W. Sander, C. (1983) How good are predictions of protein secondary structure? FEBS Lett. 155: 179-182.
Karle, I. L, (May 8, 1991) Balaram Pmodular Design of Synthetic Protein Mimics—Crystal-Structure of 2 7-Residue Helical Peptide Segments Linked by Epsilon-Aminocaproic Acid. Journal of the American Chemical Society 113(10): 3952-3956.
Keitel, T. et al. (1997) Crystallographic analysis of anti-p24 (HIV-1) monoclonal antibody cross-reactivity and polyspecificity. Cell 91: 811-820.
Kilby, J. M. and Eron J. J. (2003) Novel therapies based on mechanisms of HIV-1 cell entry. N Engl J Med. 348(22): 2228-38.
Klasse, P. J. et al. (1999) CD4-Chemokine receptor hybrids in human immunodeficiency virus type 1 infection. J Virol. 73(9): 7453-66.
Kolchinsky, P. (2001) Loss of a single N-linked glycan allows CD4-independent human immunodeficiency virus type 1 infection by altering the position of the gp120 V1/V2 variable loops. J Virol. 75: 3435-3443.
Kuhmann, S. E. et al. (2004) Genetic and phenotypic analyses of human immunodeficiency virus type 1 escape from a small-molecule CCR5 inhibitor. J Virol. 78(6): 2790-807.
Kurland, C. and Gallant, J. (1996) Errors of heterologous protein expression. Curr Opin Biotechnol. 7(5): 489-93.
Kwong, P. D. et al. (1998) Structure of an HIV gp120 envelope glycoprotein in complex with the CD4 receptor and a neutralizing human antibody. Nature 393(6686): 648-59.
Kwong, P. D. et al. (2000) Structures of HIV-1 gp120 envelope glycoproteins from laboratory-adapted and primary isolates. Structure 8(12): 1329-39.
Labrijn, A. F. et al. (2003) Access of antibody molecules to the conserved coreceptor binding site on glycoprotein gp120 is sterically restricted on primary human immunodeficiency virus type 1. J Virol. 77: 10557-10565.
Labrosse, B. et al. (1998) Determinants for sensitivity of human immunodeficiency virus coreceptor CXCR4 to the bicyclam AMD3100. J Virol. 72(8): 6381-8.
Lalezari, J. P. et al. (2005) T-1249 retains potent antiretroviral activity in patients who had experienced virological failure while on an enfuvirtide-containing treatment regimen. J Infect Dis. 191(7): 1155-63.
Lapham, C. K. et al. (1999) Fusion of monocytes and macrophages with HIV-1 correlates with biochemical properties of CXCR4 and CCR5. Nat Med. 5(3): 303-8.
Lee, B. et al. (1999) Epitope mapping of CCR5 reveals multiple conformational states and distinct but overlapping structures involved in chemokine and coreceptor function. J Biol Chem. 274(14): 9617-26.
   Lefkowitz, R. J. (1991) Thrombin receptor. Variations on a theme. Nature, 351(6325): 353-4.
Leonard, C. K. et al. (1990) Assignment of intrachain disulfide bonds and characterization of potential glycosylation sites of the type 1 recombinant human immunodeficiency virus envelope glycoprotein (gp120) expressed in Chinese hamster ovary cells. J Biol Chem. 265(18): 10373-82.
Lerner, D. J. et al. (1996) Agonist recognition by proteinase-activated receptor 2 and thrombin receptor. Importance of extracellular loop interactions for receptor function. J Biol Chem. 271(24): 13943-7.
Levine, S. J. (2004) Mechanisms of Soluble Cytokine Receptor Generation. The Journal of Immunology 173: 5343-5348.
Lin, P. F. et al. (2003) A small molecule HIV-1 inhibitor that targets the HIV-1 envelope and inhibits CD4 receptor binding. Proc Natl Acad Sci U.S.A. 100(19): 11013-8.
Locati, M. and Murphy, P. M. (1999) Chemokines and chemokine receptors: biology and clinical relevance in inflammation and AIDS. Annu Rev Med. 50: 425-40.
Loparev, V. N. et al. (1998) A third distinct tumor necrosis factor receptor of orthopoxviruses. Proc. Natl. Acad. Sci. U.S.A. 95: 3786.
Lusso, P. (2006) HIV and the chemokine system: 10 years later. The EMBO Journal 25: 447-456.
Luster, A. D. (1998) Chemokines—chemotactic cytokines that mediate inflammation. N Engl J Med. 338(7): p. 436-45.
Ma, Q. et al. (1998) Impaired B-lymphopoiesis, myelopoiesis, and derailed cerebellar neuron migration in CXCR4- and SDF-1-deficient mice. Proc Natl Acad Sci U.S.A. 95(16): 9448-53.
Madani, N. et al. (2004) Localized changes in the gp120 envelope glycoprotein confer resistance to human immunodeficiency virus entry inhibitors BMS-806 and #155. J Virol. 78(7): 3742-52.
Maddon, P. J. et al. (1985) The isolation and nucleotide sequence of a cDNA encoding the T cell surface protein T4: a new member of the immunoglobulin gene family. Cell 42(1): 93-104.
Maeda, K. et al. (2004) Spirodiketopiperazine-based CCR5 inhibitor which preserves CC-chemokine/CCR5 interactions and exerts potent activity against R5 human immunodeficiency virus type 1 in vitro. J Virol. 78(16): 8654-62.

Marozsan, A. J. et al. (2005) Generation and properties of a human immunodeficiency virus type 1 isolate resistant to the small molecule CCR5 inhibitor, SCH-417690 (SCH-D). Virology 338(1): 182-99.
Martin, K. A. et al. (1997) CD4-independent binding of SIV gp120 to rhesus CCR5. Science. 278(5342): 1470-3.
Metzger, T. G., et al. (1996) Application of the message-address concept to the docking of naltrexone and selective naltrexone-derived opioid antagonists into opioid receptor models. Neurochem Res. 21(11): 1287-94.
Mirzabekov, T. et al. (1999) Enhanced expression, native purification, and characterization of CCR5, a principal HIV-1 coreceptor. J Biol Chem. 274(40): 28745-50.
Mitraki, A. and King, J. (1992) Amino acid substitutions influencing intracellular protein folding pathways. FEBS Lett. 307: 20-5.
Mizukami, T. et al. (1988) Binding region for human immunodeficiency virus (HIV) and epitopes for HIV-blocking monoclonal antibodies of the CD4 molecule defined by site-directed mutagenesis. Proc Natl Acad Sci U.S.A. 85(23): 9273-7.
Moore, J. P. et al. (1992) A monoclonal antibody to CD4 domain 2 blocks soluble CD4-induced conformational changes in the envelope glycoproteins of human immunodeficiency virus type 1 (HIV-1) and HIV-1 infection of CD4+ cells. J Virol. 66(8): 4784-93.
Moore, J. P. and Sodroski J. (1996) Antibody cross-competition analysis of the human immunodeficiency virus type 1 gp120 exterior envelope glycoprotein. J Virol. 70(3): 1863-72.
Mosavi, L. K. and Peng, Z. Y. (2003) Structure-based substitutions for increased solubility of a designed protein. Protein Eng. 16: 739-45.
Moser, B. (1997) Chemokines and HIV: a remarkable synergism. Trends Microbiol. 5(3): 88-90.
Mosier, D. E. et al. (1999) Highly potent RANTES analogues either prevent CCR5-using human immunodeficiency virus type 1 infection in vivo or rapidly select for CXCR4-using variants. J Virol. 73(5): 3544-50.
Moulard, M. et al. (2002) Broadly cross-reactive HIV-1-neutralizing human monoclonal Fab selected for binding to gp120-CD4-CCR5 complexes. Proc Natl Acad Sci U.S.A. 99: 6913-6918.
Murby, M. et al. (1995) Hydrophobicity engineering to increase solubility and stability of a recombinant protein from respiratory syncytial virus. Eur J Biochem. 230: 38-44.
Murray, J. L. et al. (2002) Role of CD4 hinge region in GP120 utilization by immunoglobulin domain 1. Biochem Biophys Res Commun. 292(2): 449-55.
Myszka, D. G. et al. (2000) Energetics of the HIV gp120-CD4 binding reaction. Proc Natl Acad Sci U.S.A. 97(16): 9026-31.
Nagasawa, T. et al. (1996) Defects of B-cell lymphopoiesis and bone-marrow myelopoiesis in mice lacking the CXC chemokine PBSF/SDF-1. Nature 382(6592): 635-8.
Nanevicz, T. et al. (1996) Thrombin receptor activating mutations. Alteration of an extracellular agonist recognition domain causes constitutive signaling. J Biol Chem. 271(2): 702-6.
Novick, D., et al. (1999) Interleukin-18 binding protein: a novel modulator of the Th1 cytokine response. Immunity 10: 127.
O'Hara, B. M. and Olson, W. C. (2002) HIV entry inhibitors in clinical development. Curr Opin Pharmacol. 2(5): 523-8.
Olah, M. E. (1994) Role of the second extracellular loop of adenosine receptors in agonist and antagonist binding. Analysis of chimeric A1/A3 adenosine receptors. J Biol Chem. 269(40): 24692-8.
Olson, W. C. et al. (1999) Differential inhibition of human immunodeficiency virus type 1 fusion, gp120 binding, and CC-chemokine activity by monoclonal antibodies to CCR5. J. Virol. 73(5): 4145-55.
Olsen, N. J. et al. (2004) New drugs for rheumatoid arthritis. N. Engl. J. Med. 350: 2167.
Panus, J. F. et al. (2002) Cowpox virus encodes a fifth member of the tumor necrosis factor receptor family: a soluble, secreted CD30 homologue. Proc. Natl. Acad. Sci. U.S.A. 99: 8348.

Picard, L. et al. (1997) Role of the amino-terminal extracellular domain of CXCR-4 in human immunodeficiency virus type 1 entry. Virology 231(1): 105-11.
Prinz, W. A. et al. (1997) The role of the thioredoxin and glutaredoxin pathways in reducing protein disulfide bonds in the Escherichia coli cytoplasm. J Biol Chem, 272(25): 15661-7.
Poveda, E. et al. (2005) Dynamics of enfuvirtide resistance in HIV-infected patients during and after long-term enfuvirtide salvage therapy. J Clin Virol. 34(4): 295-301.
Poveda, E. et al. (2004) Evolution of genotypic and phenotypic resistance to Enfuvirtide in HIV-infected patients experiencing prolonged virologic failure. J Med Virol. 74(1): 21-8.
Ramagopal, U. A. et al. (Jan. 30, 2001) De novo design and characterization of an apolar helical hairpin peptide at atomic resolution: Compaction mediated by weak interactions. PNAS 98(3): 870-874.
Raport, C. J. et al. (1996) Molecular cloning and functional characterization of a novel human CC chemokine receptor (CCR5) for RANTES, MIP-1beta, and MIP-1alpha. J Biol Chem. 271(29): 17161-6.
Reeves, J. D. et al. (1999) Primary human immunodeficiency virus type 2 (HIV-2) isolates infect CD4-negative cells via CCR5 and CXCR4: comparison with HIV-1 and simian immunodeficiency virus and relevance to cell tropism in vivo. J Virol. 73: 7795-7804.
Reeves, J. D. et al. (2002) Sensitivity of HIV-1 to entry inhibitors correlates with envelope/coreceptor affinity, receptor density, and fusion kinetics. Proc Natl Acad Sci U.S.A. 99(25): 16249-54.
Robertson, D. (2003) US FDA approves new class of HIV therapeutics. Nat Biotechnol. 21(5): 470-1.
Rojo, D. et al. (1999) Structural biology of chemokine receptors. Biol Res. 32(4): 263-72.
Rubin, L. A. (1985) Soluble interleukin 2 receptors are released from activated human lymphoid cells in vitro. J. Immunol. 135:3172.
Rudenko, G. (1995) Three-dimensional structure of the human ‘protective protein’: structure of the precursor form suggests a complex activation mechanism. Structure 3:1249-1259.
Ryu, S. E. et al. (1990) Crystal structure of an HIV-binding recombinant fragment of human CD4. Nature 348(6300): 419-26.
Sachdev, D. and Chirgwin, J. M. (1998) Solubility of proteins isolated from inclusion bodies is enhanced by fusion to maltose-binding protein or thioredoxin. Protein Expr Purif 12: 122-32.
Samson, M. et al. (1996) Molecular cloning and functional expression of a new human CC-chemokine receptor gene. Biochemistry 35(11): 3362-7.
Saraiva, M. et al. (2001) CrmE, a novel soluble tumor necrosis factor receptor encoded by poxviruses. J. Virol. 75:226.
Saraiva, M. et al. (2002) Inhibition of type 1 cytokine-mediated inflammation by a soluble CD30 homologue encoded by ectromelia (mousepox) virus. J. Exp. Med. 196: 829.
Sattentau, Q. J. et al. (1986) Epitopes of the CD4 antigen and HIV infection. Science 234(4780): 1120-3.
Scarlatti, G. et al. (1997) In vivo evolution of HW-1 co-receptor U.S.A.ge and sensitivity to chemokine-mediated suppression. Nat Med. 3: 1259-1265.
Schein, C. H. et al. (1988) Formation of Soluble Recombinant Proteins in Escherichia Coli is Favored by Lower Growth Temperature. Bio/Technology 6: 291-294.
Schols, D. et al. (1998) T-cell-line-tropic human immunodeficiency virus type 1 that is made resistant to stromal cell-derived factor 1alpha contains mutations in the envelope gp120 but does not show a switch in coreceptor use. J Virol. 1998. 72(5): 4032-7.
Schwartz, T. W. and Rosenkilde, M. M. (1996) Is there a ‘lock’ for all agonist ‘keys’ in 7TM receptors? Trends Pharmacol Sci. 17(6): 213-6.
Seidel, H. M. et al. (1992) Phosphonate biosynthesis: molecular cloning of the gene for phosphoenolpyruvate mutase from Tetrahymena pyriformis and overexpression of the gene product in Escherichia coli. Biochemistry 31(9): 2598-608.
Sidhu, G. et al. (1999) Sugar ring distortion in the glycosyl-enzyme intermediate of a family G/11 xylanase. Biochemistry 38: 5346-5354.
Simmons, G. et al. (1997) Potent inhibition of HIV-1 infectivity in macrophages and lymphocytes by a novel CCR5 antagonist. Science 276(5310): 276-9.
Sista, P. R. et al. (2004) Characterization of determinants of genotypic and phenotypic resistance to enfuvirtide in baseline and on-treatment HIV-1 isolates. Aids 18(13): 1787-94.
Smith, C. A. et al. (1990) A receptor for tumor necrosis factor defines an unusual family of cellular and viral proteins. Science 248: 1019.
Smith, C. A. (1996) Cowpox virus genome encodes a second soluble homologue of cellular TNF receptors, distinct from CrmB, that binds TNF but not LT. Virology 223: 132.
Spriggs, M. K. et al. (1992) Vaccinia and cowpox viruses encode a novel secreted interleukin-1-binding protein. Cell 71: 145.
Strandberg, L. and Enfors, S. O. (1991) Factors influencing inclusion body formation in the production of a fused protein in Escherichia coli. Appl Environ Microbiol. 57: 1669-74.
Symons, J. A. et al. (1995) Vaccinia virus encodes a soluble type I interferon receptor of novel structure and broad species specificity. Cell 81:551.
Takeda, S. et al. (2001) Design of an artificial light-harvesting unit by protein engineering: cytochrome b(562)-green fluorescent Protein chimera. Biochem. Biophys. Res. Commun. 289: 299-304.
Taylor, W. R. (1999) Protein structural domain identification. Protein Eng. 12: 203-216.
Thoden, J. B. et al. (1998) Dramatic differences in the binding of UDP-galactose and UDP-glucose to UDP-galactose 4-epimerase from Escherichia coli. Biochemistry 37: 11469-11477.
Tremblay, C. L. et al. (2000) Strong in vitro synergy between the fusion inhibitor T-20 and the CXCR4 blocker AMD-3100. J Acquir Immune Defic Syndr. 25(2): 99-102.
Trkola, A. et al. (1999) A cell line-based neutralization assay for primary human immunodeficiency virus type 1 isolates that use either the CCR5 or the CXCR4 coreceptor. J Virol. 73(11): 8966-74.
Trkola, A. et al. (2001) Potent, broad-spectrum inhibition of human immunodeficiency virus type 1 by the CCR5 monoclonal antibody PRO140. J Virol. 75(2): 579-88.
Trkola, A. et al. (2002) HIV-1 escape from a small molecule, CCR5-specific entry inhibitor does not involve CXCR4 use. Proc Natl Acad Sci U.S.A. 99(1): 395-400.
Trumpfheller, C. et al. (1998) Expression of macrophage inflammatory protein (MIP)-1, MIP-1, and RANTES genes in lymph nodes from HIV+ individuals: correlation with a Th1-type cytokine response. Clin Exp Immunol. 112: 92-99.
Tsamis, F. et al. (2003) Analysis of the mechanism by which the small-molecule CCR5 antagonists SCH-351125 and SCH-350581 inhibit human immunodeficiency virus type 1 entry. J Virol. 77(9): 5201-8.

Tsimanis, A. et al. (Jan. 15, 2005) Soluble chemokine CCR5 receptor is present in human plasma. Immunology Letters 96(1): 55-61.
Upton, C. et al. (1991) Myxoma virus expresses a secreted protein with homology to the tumor necrosis factor receptor gene family that contributes to viral virulence. Virology 184: 370.
Upton, C. et al. (1992) Encoding of a homolog of the IFN-receptor by myxoma virus. Science 258: 1369.
Vermeire, K. et al. (2002) CADA inhibits human immunodeficiency virus and human herpesvirus 7 replication by down-modulation of the cellular CD4 receptor. Virology 302(2): 342-53.
Vermeire, K. et al. (2003) The Anti-HIV potency of cyclotriazadisulfonamide analogs is directly correlated with their ability to down-modulate the CD4 receptor. Mol Pharmacol. 63(1): 203-10.
Walker, P. et al. (1994), Acidic residues in extracellular loops of the human Y1 neuropeptide Y receptor are essential for ligand binding. J Biol Chem. 269(4): 2863-9.
Wall, J. G. and Pluckthun, A. (1995) Effects of overexpressing folding modulators on the in vof heterologous proteins in Escherichia coli. Curr Opin Biotechnol. 6: 507-16.
Wang, J. H. et al. (1990) Atomic structure of a fragment of human CD4 containing two immunoglobulin-like domains. Nature 348(6300): 411-8.
Wang, X. and Staudinger, R. (2003) Interaction of soluble CD4 with the chemokine receptor CCR5. Biochem Biophys Res Commun. 307(4): 1066-9.
Watson, C. et al. (2005) The CCR5 receptor-based mechanism of action of 873140, a potent allosteric noncompetitive HIV entry inhibitor. Mol Pharmacol. 67(4): 1268-82.
Wei, X. et al. (2002) Emergence of resistant human immunodeficiency virus type 1 in patients receiving fusion inhibitor (T-20) monotherapy. Antimicrob Agents Chemother. 46(6): 1896-905.
Weiss, C. D. (2003) HIV-1 gp41: mediator of fusion and target for inhibition. AIDS Rev. 5(4): 214-21.
Wild, C. et al. (1992) A synthetic peptide inhibitor of human immunodeficiency virus replication: correlation between solution structure and viral inhibition. Proc Natl Acad Sci U.S.A. 89(21): 10537-41.
Wild, C. et al (1993) A synthetic peptide from HIV-1 gp41 is a potent inhibitor of virus-mediated cell-cell fusion. AIDS Res Hum Retroviruses 9(11): 1051-3.
Wild, C. et al. (1994) Peptides corresponding to a predictive alpha-helical domain of human immunodeficiency virus type 1 gp41 are potent inhibitors of virus infection. Proc Natl Acad Sci U.S.A. 91(21): 9770-4.
Wu, L. et al. (1997) CCR5 levels and expression pattern correlate with infectability by macrophage-tropic HIV-1, in vitro. J Exp Med. 185(9): 1681-91.
Wu, L. et al. (1997) Interaction of chemokine receptor CCR5 with its ligands: multiple domains for HIV-1 gp120 binding and a single domain for chemokine binding. J Exp Med. 186(8): 1373-81.
Wu, H. et al. (1997) Dimeric association and segmental variability in the structure of human CD4. Nature 387(6632): 527-30.
Wyatt, R. et al. (1998) The antigenic structure of the HIV gp120 envelope glycoprotein. Nature 393(6686): 705-11.
Xiao, X. et al. (1999) Constitutive cell surface association between CD4 and CCR5. Proc Natl Acad Sci U.S.A., 96(13): 7496-501.
Yang, O. O. et al. (1997) Suppression of human immunodeficiency virus type 1 replication by CD8+ cells: evidence for HLA class I-restricted triggering of cytolytic and noncytolytic mechanisms. J Virol. 71: 3120-3128.
Zerhouni, B. et al. (2004) Isolation of CD4-independent primary human immunodeficiency virus type 1 isolates that are syncytium inducing and acutely cytopathic for CD8+ lymphocytes. J Virol. 78: 1243-1255.
Zou, Y. R. et al. (1998) Function of the chemokine receptor CXCR4 in haematopoiesis and in cerebellar development. Nature 393(6685): 595-9.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A soluble polypeptide that retains three dimensional conformation of a native G protein-coupled receptor (GPCR) protein comprising:
at least two extracellular domains of a GPCR protein, linked in tandem by short inter-domain linkers to form a soluble polypeptide that retains three dimensional conformation of a native GPCR corresponding to the extracellular domains of the GPCR protein, wherein the soluble polypeptide is capable of binding a ligand or functionally equivalent analog thereof for the native GPCR protein.

2. The soluble polypeptide of claim 1, wherein the GPCR protein is CCR5.

3. The soluble polypeptide of claim 1, wherein the GPCR protein is CXCR4.

4. The soluble polypeptide of claim 1, further comprising a tag.

5. The soluble polypeptide of claim 4, wherein the tag is a His6 tag.

6. The soluble polypeptide of claim 4, wherein the polypeptide comprises a carboxyl-tag.

7. The soluble polypeptide of claim 4, wherein the polypeptide comprises an amino-tag.

8. The soluble polypeptide of claim 4, wherein the polypeptide comprises a carboxyl- and an amino-tags.

9. The soluble peptide of claim 1, wherein the peptide linker comprises a proline and a hydrophobic amino acid.

10. The soluble peptide of claim 1, wherein the peptide linker is PGGS (SEQ ID NO:1).

11. The soluble polypeptide of claim 1, wherein the peptide linker is selected from the group consisting of: PGGS (SEQ ID NO:1), PGGGS (SEQ ID NO:2), and PGGG (SEQ ID NO:3).

12. The soluble polypeptide of claim 1, wherein the native GPCR protein is CCR5 protein comprising:
at least four domains comprising at least a portion of an N-terminal domain, an ECL1 domain, an ECL2 domain, and an ECL3 domain of CCR5, wherein each of the four domains is linked in tandem by a PGGS inter-domain linker.

13. The soluble polypeptide of claim 1, wherein the native GPCR protein is CCR5 protein comprising:
at least four domains comprising at least a portion of an N-terminal domain, an ECL1 domain, an ECL2 domain, and an ECL3 domain of CCR5, wherein each of the four domains is linked in tandem by an inter-domain linker, and at least one of the inter-domain linkers is a PGGS linker.

14. The soluble polypeptide of claim 12 or 13, further comprising one or more tags.

15. The soluble polypeptide of claim 1, wherein the native GPCR protein is CXCR4 protein comprising:
at least four domains comprising at least a portion of an N-terminal domain, an ECL1 domain, an ECL2 domain, and an ECL3 domain of CXCR4, wherein each of the four domains is linked in tandem by a PGGS inter-domain linker.

16. The soluble polypeptide of claim 1, wherein the native GPCR protein is CXCR4 protein comprising:
at least four domains comprising at least a portion of an N-terminal domain, an ECL1 domain, an ECL2 domain, and an ECL3 domain of CXCR4, wherein each of the four domains is linked in tandem by an inter-domain linker, and at least one of the inter-domain linkers is a PGGS linker.

17. The soluble polypeptide of claim 15 or 16, further comprising one or more tags.

18. The soluble polypeptide of claim 1 further comprising a CD4 N-terminal immunoglobulin variable region-like domain and a gp41 ectodomain, wherein the at least two extracellular domains of a GPCR protein, the CD4 N-terminal immunoglobulin variable region-like domain and the gp41 ectodomain are linked in tandem by short peptide linkers.

19. The soluble polypeptide of claim 1, further comprising a CD4 N-terminal immunoglobulin variable region-like domain, wherein the at least two extracellular domains of a GPCR protein and the CD4 N-terminal immunoglobulin variable region-like domain are linked in tandem by a short peptide linker.

20. The soluble polypeptide of claim 1, further comprising a gp41 ectodomain, wherein the at least two extracellular domains of a GPCR protein and the gp41 ectodomain are linked in tandem by a short peptide linker.

21. The soluble polypeptide of claim 18 or 19, wherein the CD4 N-terminal immunoglobulin variable region-like domain comprises D1 and D2 domains.

22. The soluble polypeptide of claim 18 or 20, wherein the gp41 ectodomain comprises a C-terminal intramolecular interaction domain.

23. The soluble polypeptide of claim 22, wherein the C-terminal intramolecular interaction domain is a polypeptide corresponding to amino acid residues 628-683 of gp41.

24. The soluble polypeptide of claim 18 or 20, wherein the gp41 ectodomain comprises an N-terminal intramolecular interaction domain.

25. A nucleic acid encoding the soluble polypeptide of claim 1.

26-38. (canceled)

39. A method for identifying a molecule that binds to a G protein-coupled receptor (GPCR) protein of native conformation, the method comprising:
contacting a sample containing at least one test molecule with a soluble polypeptide that retains three dimensional conformation of a native GPCR protein, comprising extracellular domains of a GPCR protein, linked in tandem by short inter-domain linkers, wherein the soluble polypeptide retains three dimensional conformation of the native GPCR, and
identifying the molecule that binds to the polypeptide.

40-67. (canceled)

68. A method of inhibiting ligand-dependent receptor stimulation of a G protein-coupled receptor (GPCR), the method comprising:
contacting a cell expressing the GPCR on the cell surface with a soluble polypeptide that retains three dimensional conformation of a native GPCR protein comprising:
at least two extracellular domains of a GPCR protein, linked in tandem by short inter-domain linkers to form a soluble polypeptide that retains three dimensional conformation of a native GPCR corresponding to the extracellular domains of the GPCR protein, wherein the soluble polypeptide is capable of binding a ligand or functionally equivalent analog thereof for the native GPCR protein in an amount effective for inhibiting ligand-dependent receptor stimulation of a GPCR.

69-71. (canceled)

72. A method of treating an HIV infection, the method comprising:
administering to a subject in need of such treatment a composition comprising a soluble polypeptide that retains native three-dimensional conformation of extracellular portions of an HIV co-receptor, comprising at least part of an N-terminus, an ECL1 domain, an ECL2 domain and an ECL3 domain of the HIV co-receptor, linked in tandem by inter-domain PGGS linkers and disulfide bonds, in a pharmacologically effective amount to treat HIV infection.

73-81. (canceled)

82. A method of treating a disease or disorder caused by a G protein-coupled receptor (GPCR) mutation, wherein the GPCR mutation is associated with altered basal activity, the method comprising:
administering to a subject in need of such treatment a composition comprising a soluble polypeptide that retains native three-dimensional conformation of extracellular portions of a GPCR, comprising at least part of an N-terminus, an ECL1 domain, an ECL2 domain and an ECL3 domain of the GPCR, linked in tandem by inter-domain linkers, wherein at least one of the inter-domain linkers comprises a PGGS linker, and disulfide bonds, in an amount effective to treat the disease or disorder caused by a GPCR mutation.

83. The method of claim 82, wherein the disease or disorder is selected from the group consisting of:
Congenital night blindness, Retinitis pigmentosa, Gastric carcinoid tumors, Kaposi's sarcoma, primary effusion lymphoma, Atherosclerosis, infections in immunocompromised patients, Adenoma or hyperplasia associated with hyperthyroidism, Male precocious puberty, Leydig cell tumor associated with male precocious puberty, Normal semen parameters despite hypophysectomy, ansen-type metaphyseal chondrodysplasia (dwarfism, hypercalcemia, hypophosphatemia, Autosomal dominant hypocalcemia.

84-102. (canceled)
